(19) United States
(12) Patent Application Publication
(10) Pub. No.: US 2025/0186411 A1
(43) Pub. Date: Jun. 12, 2025

(54) Methods of Treating Disorders Associated with Overactivation of IKKB

(71) Applicants: The United States Government as represented by the Department of Veterans Affairs, Washington, DC (US); The Research Foundation for the State University of New York, Albany, NY (US); Icahn School of Medicine at Mount Sinai, New York, NY (US)

(72) Inventors: Sandeep Mallipattu, St. James, NY (US); Bhaskar Das, West Nyack, NY (US)

(21) Appl. No.: 18/970,638

(22) Filed: Dec. 5, 2024

Related U.S. Application Data
(60) Provisional application No. 63/606,957, filed on Dec. 6, 2023.

(51) Int. Cl. A61K 31/437; A61K 31/167; A61K 31/4245; A61K 31/44; A61K 31/69; A61K 45/06

(52) U.S. Cl. CPC A61K 31/437; A61K 31/167; A61K 31/4245; A61K 31/44; A61K 31/69; A61K 45/06

(57) ABSTRACT

The present disclosure is concerned with methods of treating disorders associated with overactivation of IKKβ such as, for example, cancer, arthritis, a cardiometabolic disease, and chronic obstructive pulmonary disease (COPD). This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/606,957, filed on Dec. 6, 2023, the contents of which are incorporated herein by reference in their entirety.

### STATEMENT REGARDING FEDERALLY SPONSORED RESEARCH

This invention was made with government support under grant number 1I01BX005300 and 1I01BX003698 awarded by the Department of Veterans Affairs and grant number DK102519 awarded by the National Institutes of Health. The government has certain rights in the invention.

### BACKGROUND

Chronic kidney disease (CKD) is a leading risk factor for cardiovascular disease (Chronic Kidney Disease in the United States, (2023). Atlanta, GA: US Department of Health and Human Services, Centers for Disease Control and Prevention). The primary etiology of CKD is a direct consequence of initial dysfunction and injury to the glomerulus, the kidney filtration barrier. Podocytes (visceral epithelial cells) in normal mature glomerulus are regarded as highly differentiated and quiescent cells. In many glomerular diseases such as Focal Segmental Glomerulosclerosis (FSGS) and HIV-associated nephropathy (HIVAN), podocytes are injured (A. Meyrier, (2005) Nature clinical practice. Nephrology 1, 44-54) and undergo a major change in phenotype resulting in a loss of podocyte cytoskeleton, actin stress fiber formation, and their terminal differentiation markers (L. Barisoni, (2012) Adv Chronic Kidney Dis 19, 76-83).

Glucocorticoids (GCs) are the initial treatment option for many glomerular diseases, such as Minimal Change Disease (MCD) and FSGS M. van Husen, M. J. Kemper, (2011) Pediatr Nephrol 26, 881-892). In many instances, alternate immunosuppressive therapy is typically not considered until patients have failed GC therapy. Although GCs can be effective at times, it has been challenging for nephrologists to determine early which individuals are likely to respond to GC therapy. For instance, treatment with FSGS requires a minimum of 16 weeks of GC therapy (C. Ponticelli, et al., (2018)Clin J Am Soc Nephrol 13, 815-822), thereby resulting in their prolonged use leading to systemic adverse effects, ranging from weight gain, hyperglycemia, and systemic infections. While the immunomodulatory effects are GCs are important (P. J. Barnes, (1998) Clin Sci (Lond) 94, 557-572, C. Riccardi, et al., (2002) Pharmacol Res 45, 361-368), several studies demonstrated that glucocorticoid receptor (GR) as well as the major components of GR complex are expressed in human podocytes and, as such, have a direct salutary effect on the podocyte by rearrangement of actin cytoskeleton, inhibiting apoptosis, and regulating protein trafficking of critical slit diaphragm proteins (E. Schonenberger, et al., (2011) Nephrol Dial Transplant 26, 18-24, A. Guess et al., (2010) Am J Physiol Renal Physiol 299, F845-853, T. Wada, et al., (2008) Nephron Exp Nephrol 109, e8-19, T. Wada, et al., (2005). J Am Soc Nephrol 16, 2615-2625, C. Y. Xing et al., (2006) Kidney Int 70, 1038-1045, H. Zhou et al., (2017) Sci Rep 7, 9833). While the identification of novel mediators has contributed significantly to the understanding of the mechanisms mediating podocytopathy in glomerular diseases such as FSGS (S. K. Mallipattu, J. C. He, (2016) Am J Physiol Renal Physiol 311, F46-51, E. Torban et al., (2019) Kidney Int 96, 850-861, A. S. De Vriese, et al., (2021) Nat Rev Nephrol 17, 619-630), a major gap in the field remains the dearth of novel therapeutics that are alternative and/or synergistic to GCs without systemic toxicity associated with these agents.

Krüppel-Like Factor 15 (KLF15) is an early-inducible GC-responsive gene and the knockdown of KLF15 attenuates the salutary effects of GCs in human podocytes and in murine models of proteinuric kidney disease (S. K. Mallipattu et al., (2017) J Am Soc Nephrol 28, 166-184). KLF15 belongs to a subclass of zinc-finger family of DNA-binding transcriptional regulators that are involved in a broad range of cellular processes (i.e., cell differentiation, metabolism, and inflammation) (S. K. Mallipattu, et al., (2017) Am J Physiol Renal Physiol 312, F259-F265, X. Gu et al., (2017) Kidney Int, A. B. Bialkowska, et al., (2017) Development 144, 737-754, M. J. Rane, et al., (2019) EBioMedicine 40, 743-750, L. Wang, et al., (2019) Int J Biol Sci 15, 1955-1961). Previous studies demonstrate that KLF15 is critical for the maintenance of mature podocyte differentiation markers in human podocytes and in proteinuric murine models (S. K. Mallipattu et al., (2017) J Am Soc Nephrol 28, 166-184, Y. Guo et al., (2018) J Am Soc Nephrol 29, 2529-2545, S. K. Mallipattu et al., (2012). J Biol Chem 287, 19122-19135). The podocyte-specific expression of KLF15 in human kidney biopsies also correlated with responsiveness to GCs in primary glomerulopathies such as FSGS and MCD (S. K. Mallipattu et al., (2017) J Am Soc Nephrol 28, 166-184). It was also demonstrated that the induction of podocyte-specific KLF15 ameliorated albuminuria, podocyte injury, FSGS, interstitial fibrosis, and overall kidney function in murine models of proteinuric kidney disease (Y. Guo et al., (2018) J Am Soc Nephrol 29, 2529-2545).

Despite this strong rigor of prior research, the identification and development of KLF15 agonists as therapeutics in primary glomerulopathies has remained elusive. Moreover, to the extent that KLF15 agonists have been identified, the mechanisms by which such molecules restor KLF15 function remains poorly understood. As such, the therapeutic versatility of KLF15 agonists has similarly remained limited. Accordingly, there remains a need for an improved understanding of the mechanisms mediating KLF15 activity, which understanding can be harnessed to expand the therapeutic utility of KLF15 agonists to additional diseases and disorders. These needs and others are met by the present invention.

### SUMMARY

In accordance with the purpose(s) of the invention, as embodied and broadly described herein, the invention, in one aspect, relates to methods of treating disorders associated with overactivation of IKKβ such as, for example, cancer (e.g., a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, plasma cell neoplasm (myeloma)), arthritis (e.g., osteoarthritis, psoriatic arthritis, rheumatoid arthritis, juvenile idiopathic arthritis), a cardiometabolic disease (e.g., heart attack, stroke, diabetes, insulin resistance, non-alcoholic fatty liver disease), and chronic obstructive pulmonary disease (COPD).

Disclosed are methods of treating a disorder associated with overactivation of IKKβ in a subject in need thereof, the method comprising administering to the subject a compound having a structure represented by a formula selected from:

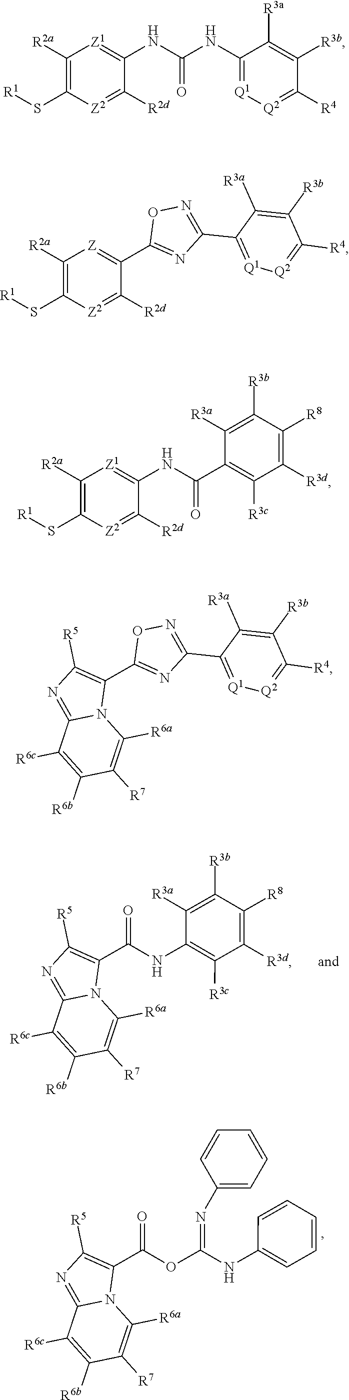

wherein each of Q¹ and Q² is independently selected from N and CR¹⁰; wherein R¹⁰, when present, is selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Z¹, when present, is selected from N and CR^{2b}; wherein Z², when present, is selected from N and CR^{2c}; wherein R¹, when present, is C1-C4 alkyl; wherein each of R^{2a}, R^{2b}, R^{2c}, R^{2d}, R^{3a}, and R^{3b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3c} and R^{3d}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is independently selected from halogen, —CN, —OH, C1-C4 alkyl, C1-C4 alkoxy, —B(OR¹¹)₂, and —B(R¹²)₃; wherein each occurrence of R¹¹, when present, is independently selected from hydrogen and C1-C8 alkyl, or wherein each occurrence of R¹¹, when present, is covalently bonded together, and, together with the intermediate atoms, comprise a C6 bicyclic heterocycle or a C2-C3 heterocycloalkyl, and is substituted with 0, 1, 2, 3, or 4 C1-C4 alkyl groups; wherein each occurrence of R¹², when present, is independently halogen; wherein each of R⁵, R^{6a}, R^{6b}, and R^{6c}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁷, when present, is halogen; or a pharmaceutically acceptable salt thereof, wherein the disorder is selected from cancer, arthritis, a cardiometabolic disease, and chronic obstructive pulmonary disease (COPD).

Also disclosed are methods for treating a disorder associated with overactivation of IKKβ in a subject in need thereof, the method comprising administering to the subject a compound selected from:

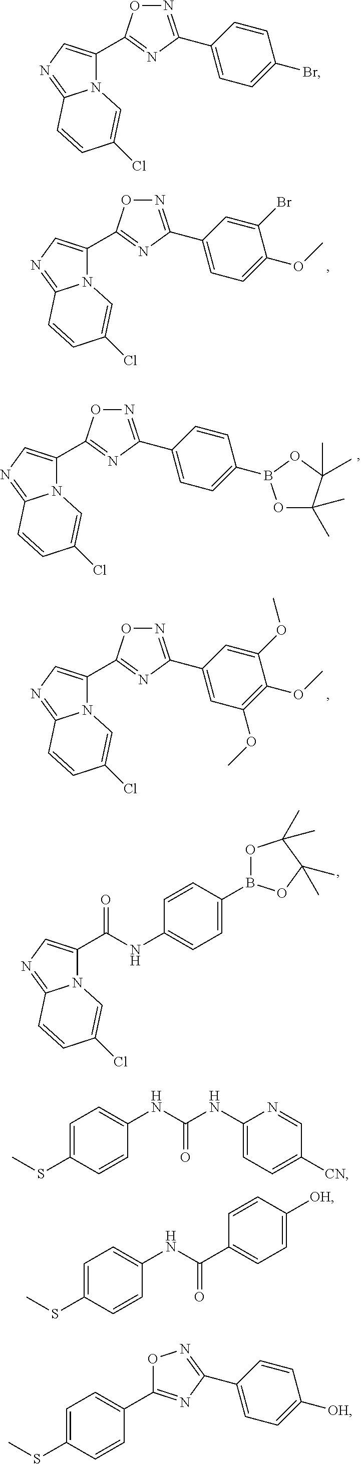

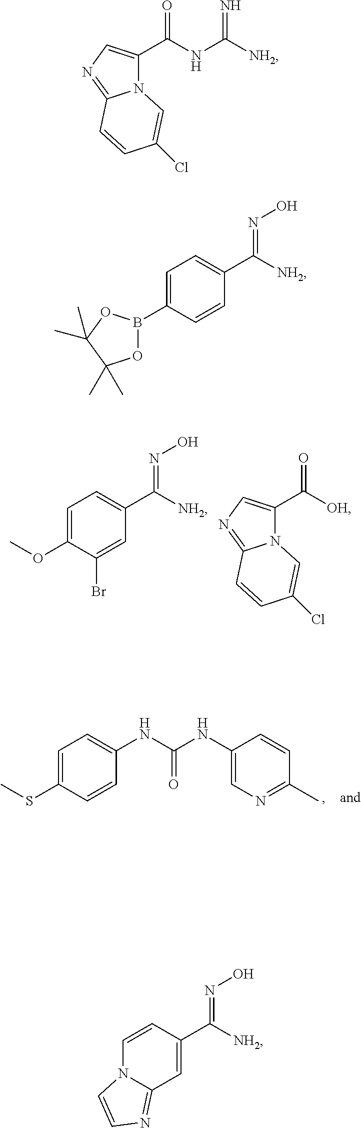

or a pharmaceutically acceptable salt thereof, wherein the disorder is selected from cancer, arthritis, a cardiometabolic disease, and chronic obstructive pulmonary disease (COPD).

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1A-F show representative data illustrating the identification of hit KLF15 agonists from NCI²⁶⁴⁵ HTS.

FIG. 2A-H show representative data illustrating that KLF15 agonists attenuate podocyte injury in the setting of podocyte stress.

FIG. 3A-G show representative compounds and synthetic schemes depicting the Structure Activity Relationship (SAR) Study of C-7 by modifying different core structural moieties.

FIG. 4A-F show representative data illustrating that lead compounds restore KLF15 activity in the setting of podocyte stress.

FIG. 5A-F show representative data illustrating that BT503 attenuates albuminuria, podocyte injury, and the dexamethasone dose in the setting of LPS treatment.

FIG. 6A-I show representative data illustrating that BT503 attenuates albuminuria, podocyte injury, and glomerulosclerosis post-NTS treatment.

FIG. 7A-P show representative data illustrating that BT503 attenuates albuminuria, podocyte injury, and glomerulosclerosis in Tg26 mice.

FIG. 8A-F show a representative data illustrating differentially expressed genes (DEGs) with enrichment analysis, which demonstrates the upregulation of genes involved in differentiation and the downregulation of genes involved in NF-κB signaling in BT503-treated podocytes.

FIG. 9A-I show representative data illustrating that BT503 increases KLF15 activity in differentiated human podocytes.

FIG. 10A-L show representative data illustrating that BT503 inhibits IKKβ activity, leading to the inhibition of p50/p65 translocation and restoration of KLF15 in the setting of cell stress.

FIG. 11A-D show representative data illustrating molecular footprints for energy-minimized 1PU, docked 1PU, and INH14 on IKKβ.

FIG. 12A-J show representative data from toxicity studies for BT503 in podocytes and mice.

FIG. 13 shows a representative synthetic scheme to access sulfoxide, sulfone, and sulfonamide derivatives.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a non-aromatic carbon-based ring type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group that has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹-, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

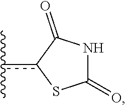

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radioactively labeled forms, isomers, solvates and combinations thereof. The “combinations” mentioned in this context are refer to derivatives falling within at least two of the groups: pharmaceutically acceptable salts, prodrugs, deuterated forms, radioactively labeled forms, isomers, and solvates. Examples of radioactively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules that owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

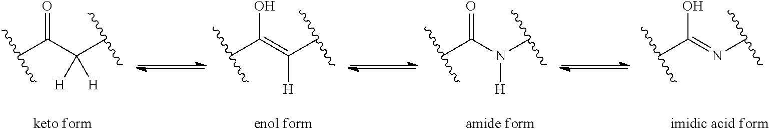 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

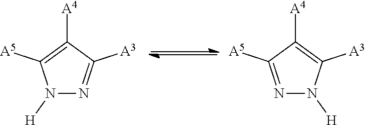

Unless stated to the contrary, the invention includes all such possible tautomers.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

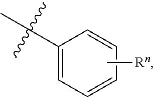

which is understood to be equivalent to a formula:

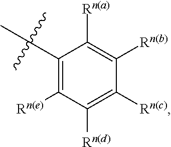

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Methods of Treating a Disorder Associated with Overactivation of IKKβ

In one aspect, disclosed are methods of treating a disorder associated with overaction of IKKβ in a subject in need thereof, the method comprising administering to the subject a disclosed compound or a pharmaceutically acceptable salt thereof, wherein the disorder is selected from cancer, arthritis, a cardiometabolic disease, and chronic obstructive pulmonary disease (COPD).

In one aspect, disclosed are methods of treating a disorder associated with overactivation of IKKβ in a subject in need thereof, the method comprising administering to the subject a compound having a structure represented by a formula selected from:

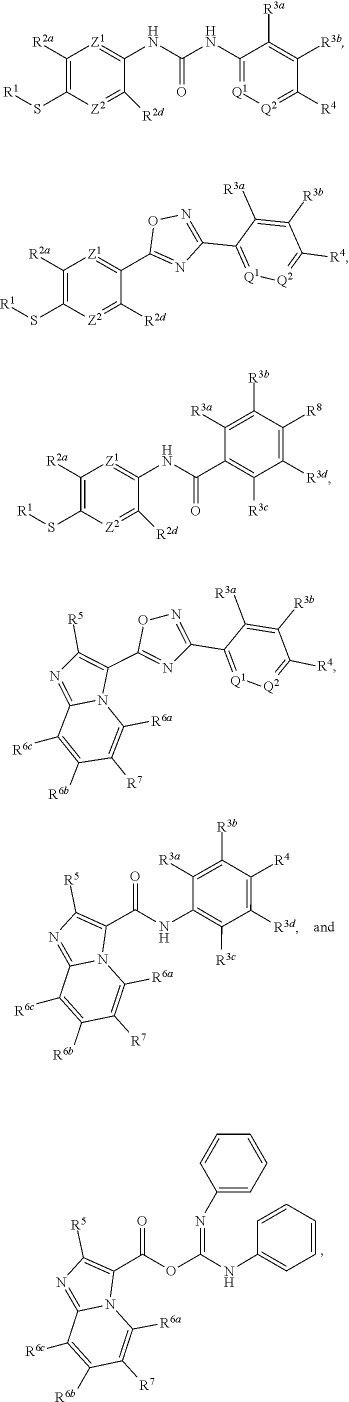

wherein each of Q¹ and Q² is independently selected from N and CR¹⁰; wherein R¹⁰, when present, is selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Z¹, when present, is selected from N and CR^{2b}; wherein Z², when present, is selected from N and CR²c; wherein R¹, when present, is C1-C4 alkyl; wherein each of R^{2a}, R^{2b}, R^{2c}, R^{2d}, R^{3a}, and R^{3b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3c} and R^{3d}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is independently selected from halogen, —CN, —OH, C1-C4 alkyl, C1-C4 alkoxy, —B(OR¹¹)₂, and —B(R¹²)₃; wherein each occurrence of R¹¹, when present, is independently selected from hydrogen and C1-C8 alkyl, or wherein each occurrence of R¹¹, when present, is covalently bonded together, and, together with the intermediate atoms, comprise a C6 bicyclic heterocycle or a C2-C3 heterocycloalkyl, and is substituted with 0, 1, 2, 3, or 4 C1-C4 alkyl groups; wherein each occurrence of R¹², when present, is independently halogen; wherein each of R⁵, R^{6a}, R^{6b}, and R^{6c}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁷, when present, is halogen; or a pharmaceutically acceptable salt thereof, wherein the disorder is selected from cancer, arthritis, a cardiometabolic disease, and chronic obstructive pulmonary disease (COPD).

In one aspect, disclosed are methods for treating a disorder associated with overactivation of IKKβ in a subject in need thereof, the method comprising administering to the subject a compound selected from:

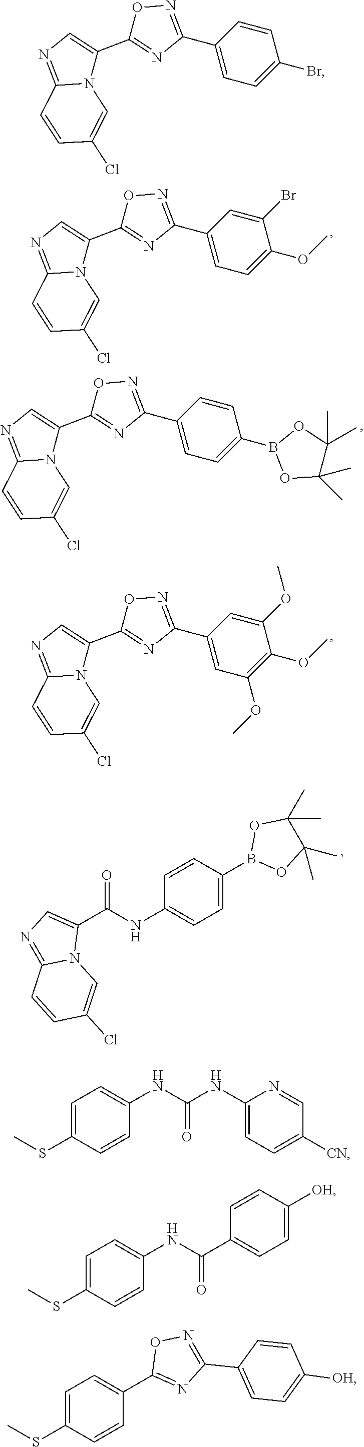

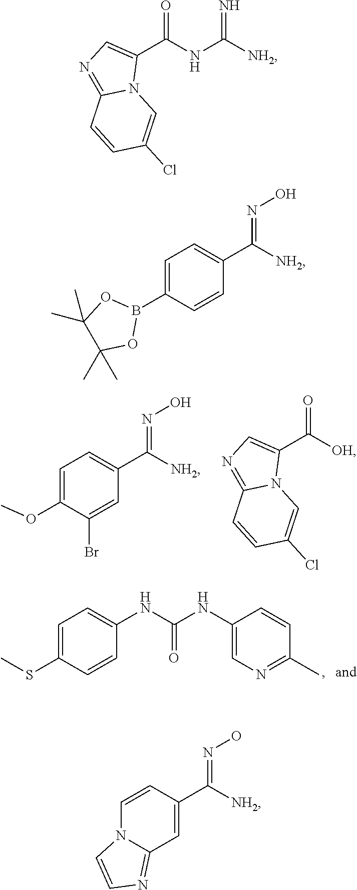

or a pharmaceutically acceptable salt thereof, wherein the disorder is selected from cancer, arthritis, a cardiometabolic disease, and chronic obstructive pulmonary disease (COPD).

In various aspects, the compound has a structure represented by a formula selected from:

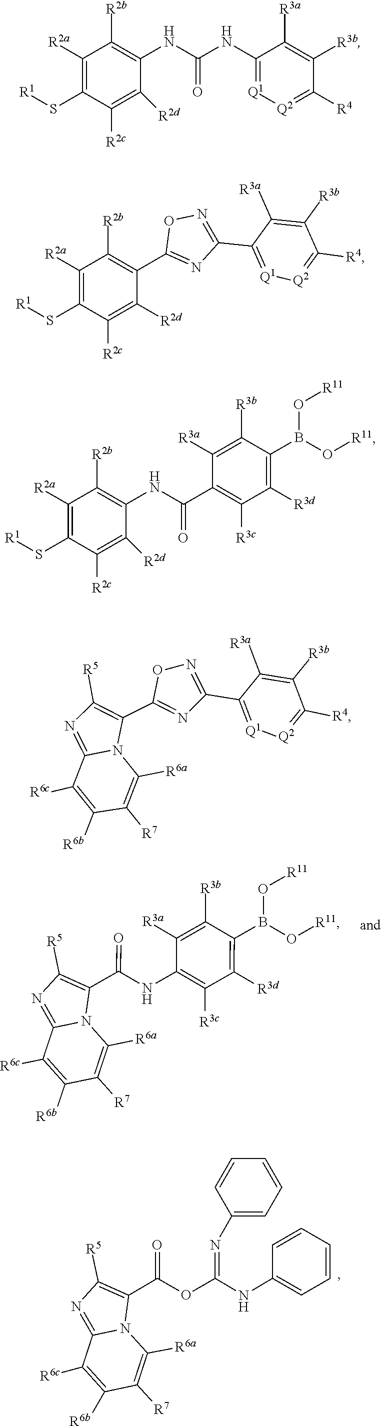

or a pharmaceutically acceptable salt thereof, wherein R⁴, when present, is independently selected from halogen, —CN, —OH, C₁-C₄ alkyl, C₁-C₄ alkoxy, and —B(OR¹¹)₂.

In various aspects, each of Q¹ and Q², when present, is CH.

In various aspects, Q¹, when present, is CH and Q², when present, is N.

In various aspects, Q¹, when present, is N and Q², when present, is CH.

In various aspects, R¹, when present, is methyl.

In various aspects, each of R^{2a}, R^{2b}, R^{2c}, R^{2d}, R^{3a}, and R^{3b}, when present, is hydrogen.

In various aspects, each of R^{3c} and R^{3d}, when present, is hydrogen.

In various aspects, R⁴, when present, is —B(OR¹¹)₂.

In various aspects, each occurrence of R¹¹, when present, is independently selected from hydrogen and C1-C8 alkyl.

In various aspects, each occurrence of R¹¹, when present, is hydrogen.

In various aspects, each occurrence of R¹¹, when present, is C1-C8 alkyl.

In various aspects, each occurrence of R¹¹, when present, is covalently bonded together, and, together with the intermediate atoms, comprise a C6 bicyclic heterocycle or a C2-C3 heterocycloalkyl, and is substituted with 0, 1, 2, 3, or 4 C1-C4 alkyl groups.

In various aspects, each occurrence of R¹¹, when present, is covalently bonded together, and, together with the intermediate atoms, comprise a C2-C3 heterocycloalkyl substituted with 0, 1, 2, 3, or 4 C1-C4 alkyl groups.

In various aspects, each occurrence of R¹¹, when present, is covalently bonded together, and, together with the intermediate atoms, comprise a C2-C3 heterocycloalkyl substituted with 4 methyl groups.

In various aspects, each of R⁵, R^{6a}, R^{6b}, and R^{6c}, when present, is hydrogen.

In various aspects, R⁷, when present, is —Cl.

In various aspects, the compound has a structure represented by a formula selected from:

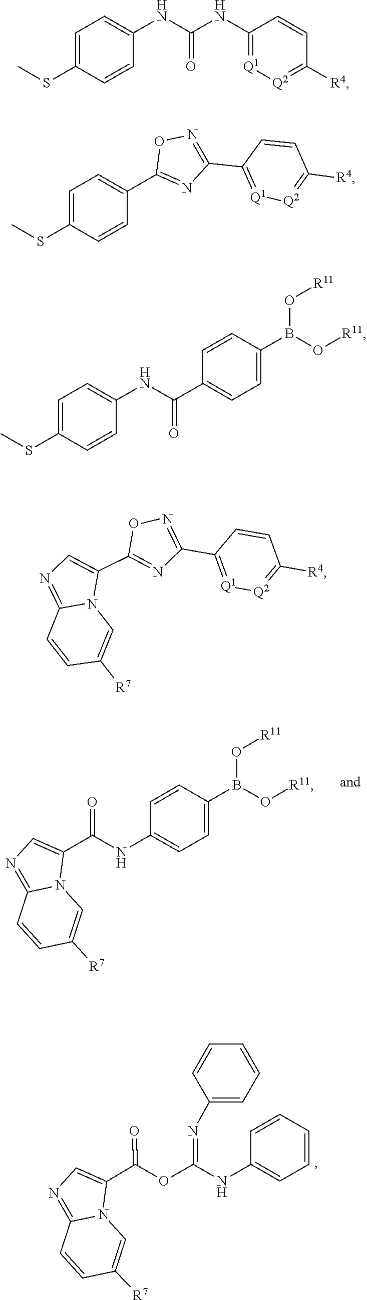

or a pharmaceutically acceptable salt thereof.

In various aspects, the compound has a structure represented by a formula selected from:

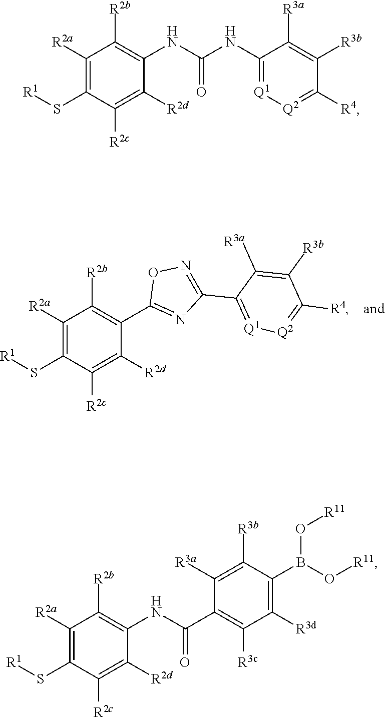

or a pharmaceutically acceptable salt thereof. In a further aspect, R¹ is methyl. In a still further aspect, each of R^{2a}, R^{2b}, R^{2c}, R^{2d}, R^{3a}, and R^{3b} is hydrogen. In yet a further aspect, R⁴, when present, is —B(OR¹¹)₂. In an even further aspect, the compound has a structure represented by a formula:

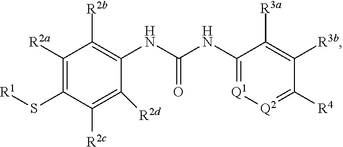

or a pharmaceutically acceptable salt thereof. In a still further aspect, the compound has a structure represented by a formula:

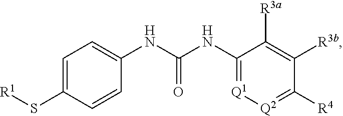

or a pharmaceutically acceptable salt thereof. In yet a further aspect, the compound has a structure represented by a formula:

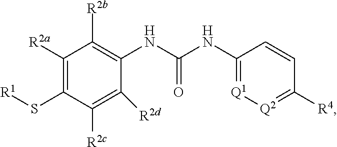

or a pharmaceutically acceptable salt thereof. In an even further aspect, the compound has a structure represented by a formula:

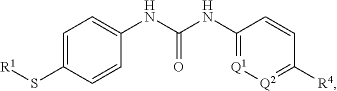

or a pharmaceutically acceptable salt thereof. In a still further aspect, the compound has a structure represented by a formula:

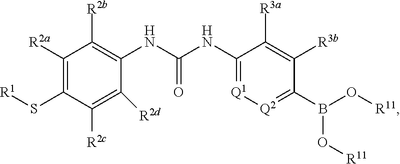

or a pharmaceutically acceptable salt thereof. In yet a further aspect, the compound is selected from:

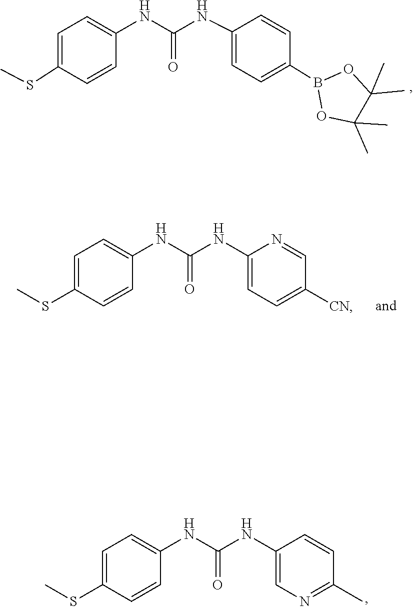

or a pharmaceutically acceptable salt thereof. In an even further aspect, the compound has a structure represented by a formula:

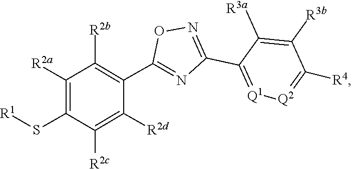

or a pharmaceutically acceptable salt thereof. In a still further aspect, the compound has a structure represented by a formula:

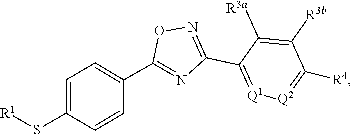

or a pharmaceutically acceptable salt thereof. In yet a further aspect, the compound has a structure represented by a formula:

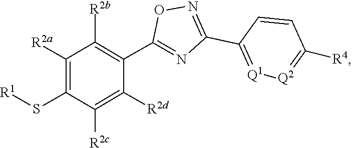

or a pharmaceutically acceptable salt thereof. In an even further aspect, the compound has a structure represented by a formula:

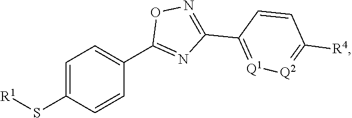

or a pharmaceutically acceptable salt thereof. In a still further aspect, the compound has a structure represented by a formula:

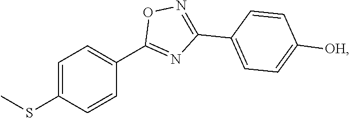 [SMILES: CSc1ccc(-c2nc(-c3ccc(O)cc3)no2)cc1]

or a pharmaceutically acceptable salt thereof. In yet a further aspect, the compound has a structure represented by a formula:

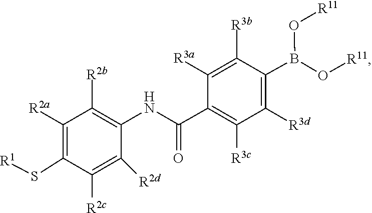

or a pharmaceutically acceptable salt thereof. In an even further aspect, the compound has a structure represented by a formula:

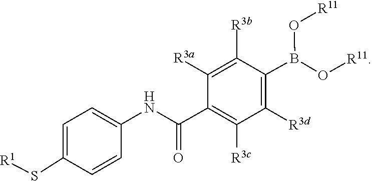

or a pharmaceutically acceptable salt thereof. In a still further aspect, the compound has a structure represented by a formula:

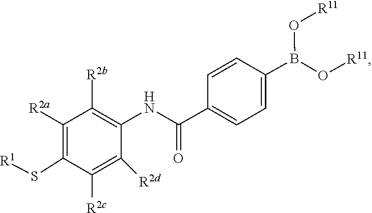

or a pharmaceutically acceptable salt thereof. In yet a further aspect, the compound has a structure represented by a formula:

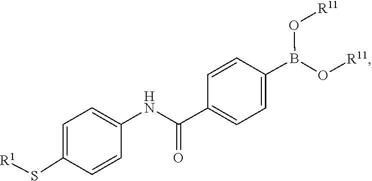

or a pharmaceutically acceptable salt thereof.

In various aspects, the compound has a structure represented by a formula selected from:

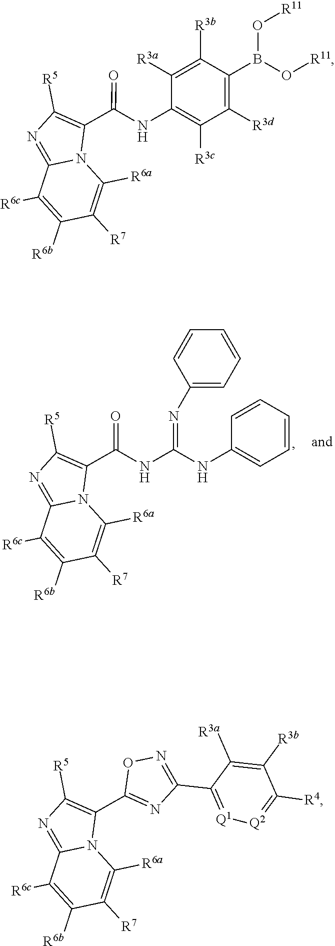

or a pharmaceutically acceptable salt thereof. In a further aspect, each of R^{3a} and R^{3b}, when present, is hydrogen. In a still further aspect, R⁴, when present, is —B(OR¹¹)₂. In yet a further aspect, each of R⁵, R^{6a}, R^{6b}, and R^{6c} is hydrogen. In an even further aspect, R⁷ is —Cl. In a still further aspect, the compound has a structure represented by a formula:

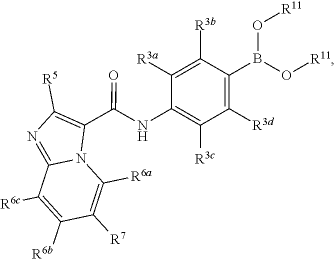

or a pharmaceutically acceptable salt thereof. In a still further aspect, the compound has a structure represented by a formula:

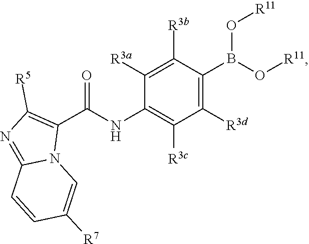

or a pharmaceutically acceptable salt thereof. In yet a further aspect, the compound has a structure represented by a formula:

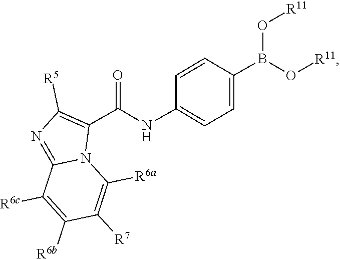

or a pharmaceutically acceptable salt thereof. In an even further aspect, the compound has a structure represented by a formula:

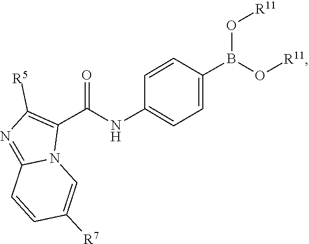

or a pharmaceutically acceptable salt thereof. In a still further aspect, the compound has a structure represented by a formula:

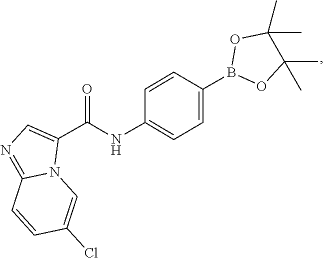 [SMILES: CC1(C)OB(c2ccc(NC(=O)c3cnc4ccc(Cl)cn34)cc2)OC1(C)C]

or a pharmaceutically acceptable salt thereof. In yet a further aspect, the compound has a structure represented by a formula:

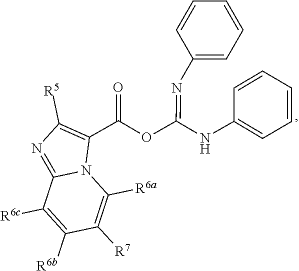

or a pharmaceutically acceptable salt thereof. In an even further aspect, the compound has a structure represented by a formula:

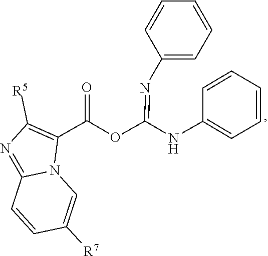

or a pharmaceutically acceptable salt thereof. In a still further aspect, the compound has a structure represented by a formula:

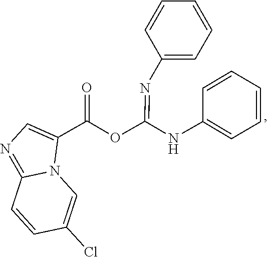 [SMILES: O=C(O/C(=N/c1ccccc1)Nc1ccccc1)c1cnc2ccc(Cl)cn12]

or a pharmaceutically acceptable salt thereof. In yet a further aspect, the compound has a structure represented by a formula:

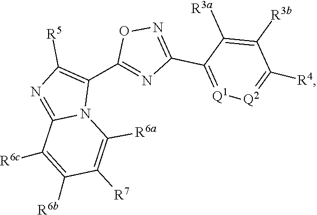

or a pharmaceutically acceptable salt thereof. In an even further aspect, the compound has a structure represented by a formula:

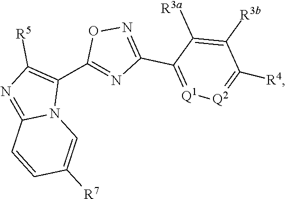

or a pharmaceutically acceptable salt thereof. In a still further aspect, the compound has a structure represented by a formula:

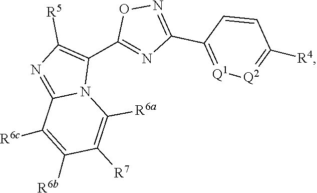

or a pharmaceutically acceptable salt thereof. In yet a further aspect, the compound has a structure represented by a formula:

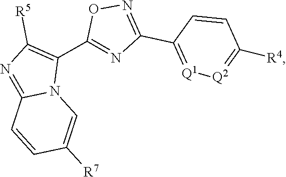

or a pharmaceutically acceptable salt thereof.

In various aspects, the compound is selected from:

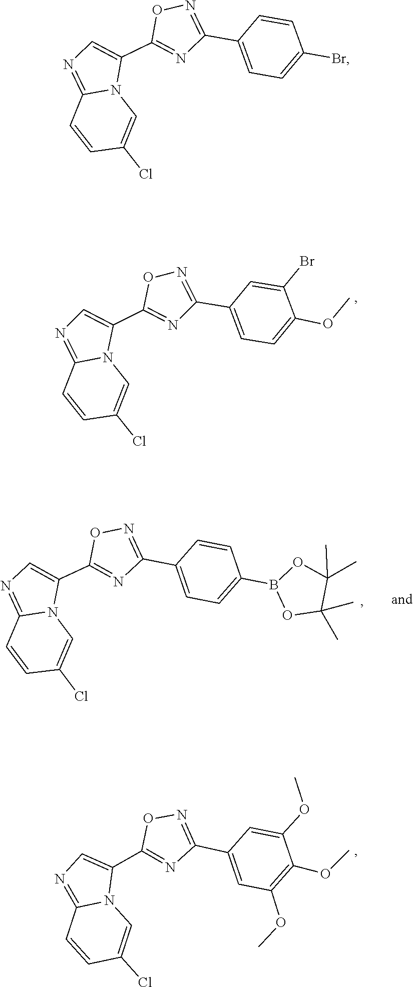 [SMILES: Clc1ccc2ncc(-c3nc(-c4ccc(Br)cc4)no3)n2c1 ; COc1ccc(-c2noc(-c3cnc4ccc(Cl)cn34)n2)cc1Br ; CC1(C)OB(c2ccc(-c3noc(-c4cnc5ccc(Cl)cn45)n3)cc2)OC1(C)C ; COc1cc(-c2noc(-c3cnc4ccc(Cl)cn34)n2)cc(OC)c1OC]

or a pharmaceutically acceptable salt thereof.

In various aspects, the compound is selected from:

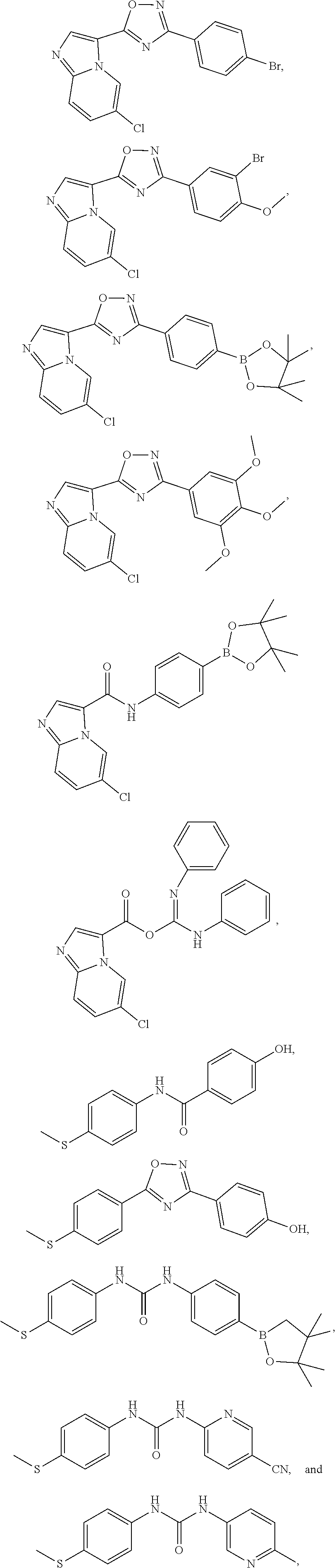

or a pharmaceutically acceptable salt thereof.

In a further aspect, the disorder is cancer. Examples of cancer include, but are not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma). In yet a further aspect, the cancer is selected from a solid tumor, a metastatic melanoma, and a hematological malignancy.

In a further aspect, the disorder is arthritis. Examples of arthritises include, but are not limited to, osteoarthritis, psoriatic arthritis, rheumatoid arthritis, and juvenile idiopathic arthritis. In a still further aspect, the arthritis is osteoarthritis.

In a further aspect, the disorder is a cardiometabolic disease. Examples of cardiometabolic diseases include, but are not limited to, heart attack, stroke, diabetes, insulin resistance, and non-alcoholic fatty liver disease.

In a further aspect, the disorder is is COPD.

In a further aspect, the effective amount is a therapeutically effective amount.

In a yet further aspect, the effective amount is a prophylactically effective amount.

In a further aspect, the subject is a mammal. In a yet further aspect, the mammal is a human.

In a further aspect, the subject has been diagnosed with a need for treatment of the disorder prior to the administering step.

In a further aspect, the subject subject is at risk for developing the disorder prior to the administering step.

In a further aspect, the method further comprises the step of identifying a subject in need of treatment of the disorder.

In a further aspect, the method further comprises administering to the subject an effective amount of a glucocorticoid. Examples of glucocorticoids include, but are not limited, to beclomethasone, betamethasone, budesonide, cortisone, dexamethasone, hydrocortisone, methylprednisolone, prednisolone, prednisone, and triamcinolone. In a yet further aspect, the glucocorticoid is dexamethasone.

In various aspects, the glucocorticoid is administered simultaneously with the compound. In various aspects, the glucocorticoid is administered sequentially before or after the compound.

In various aspects, the disorder is selected from a solid tumor, a metastatic melanoma, a hematological malignancy, osteoarthritis, and COPD.

### 1. Structure

In one aspect, the compound has a structure represented by a formula selected from:

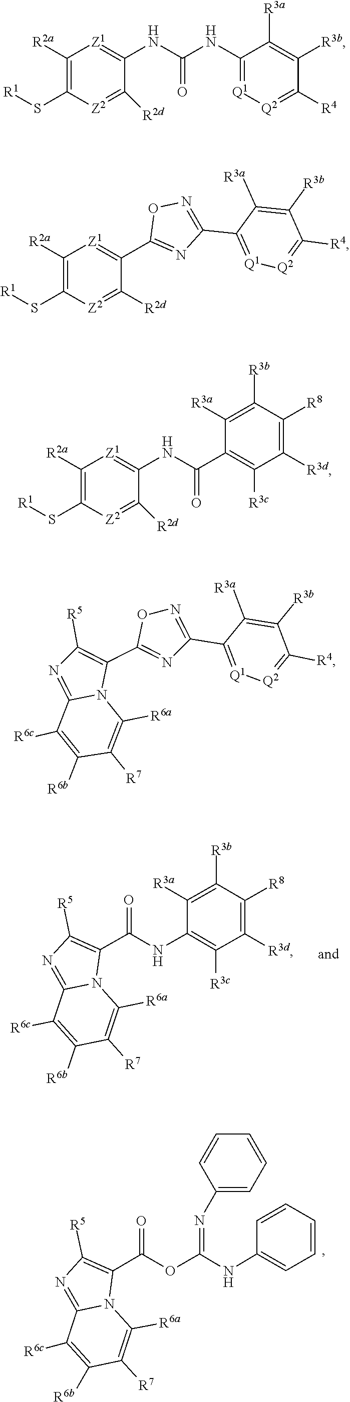

wherein each of Q¹ and Q² is independently selected from N and CR¹⁰; wherein R¹⁰, when present, is selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Z¹, when present, is selected from N and CR^{2b}; wherein Z², when present, is selected from N and CR^{2c}; wherein R¹, when present, is C1-C4 alkyl; wherein each of R^{2a}, R^{2b}, R^{2c}, R^{2d}, R^{3a}, and R^{3b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3c} and R^{3d}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is independently selected from halogen, —CN, —OH, C1-C4 alkyl, C1-C4 alkoxy, —B(OR¹¹)₂, and —B(R¹²)₃; wherein each occurrence of R¹¹, when present, is independently selected from hydrogen and C1-C8 alkyl, or wherein each occurrence of R¹¹, when present, is covalently bonded together, and, together with the intermediate atoms, comprise a C6 bicyclic heterocycle or a C2-C3 heterocycloalkyl, and is substituted with 0, 1, 2, 3, or 4 C1-C4 alkyl groups; wherein each occurrence of R¹², when present, is independently halogen; wherein each of R⁵, R^{6a}, R^{6b}, and R^{6c}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁷, when present, is halogen; or a pharmaceutically acceptable salt thereof.

In various aspects, the compound is selected from:

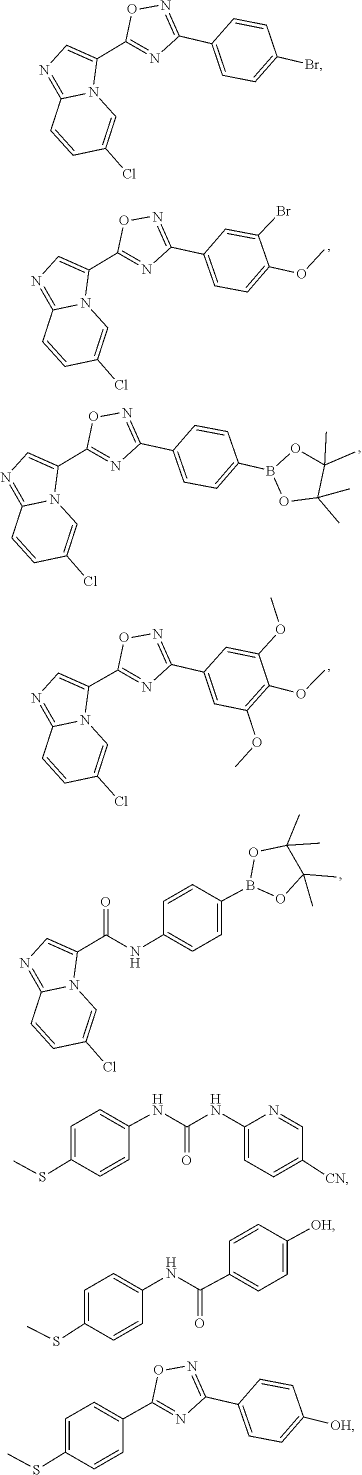

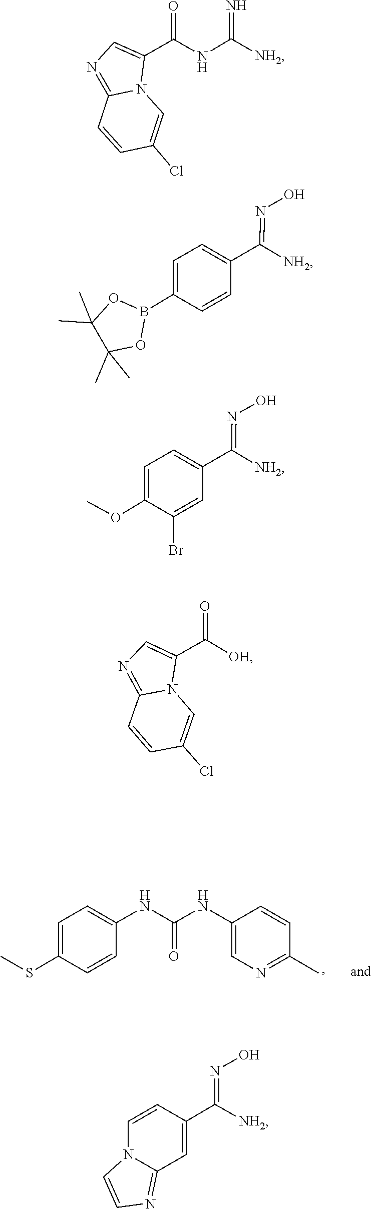

or a pharmaceutically acceptable salt thereof.

In a further aspect, the compound has a structure represented by a formula selected from:

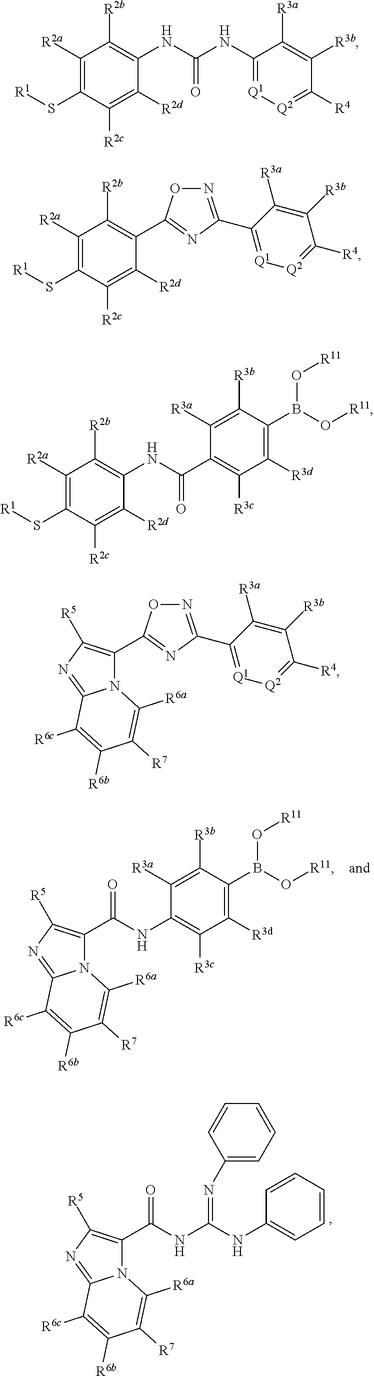

wherein R⁴, when present, is independently selected from halogen, —CN, —OH, C1-C4 alkyl, C1-C4 alkoxy, and —B(OR¹¹)₂.

In a further aspect, the compound has a structure represented by a formula selected from:

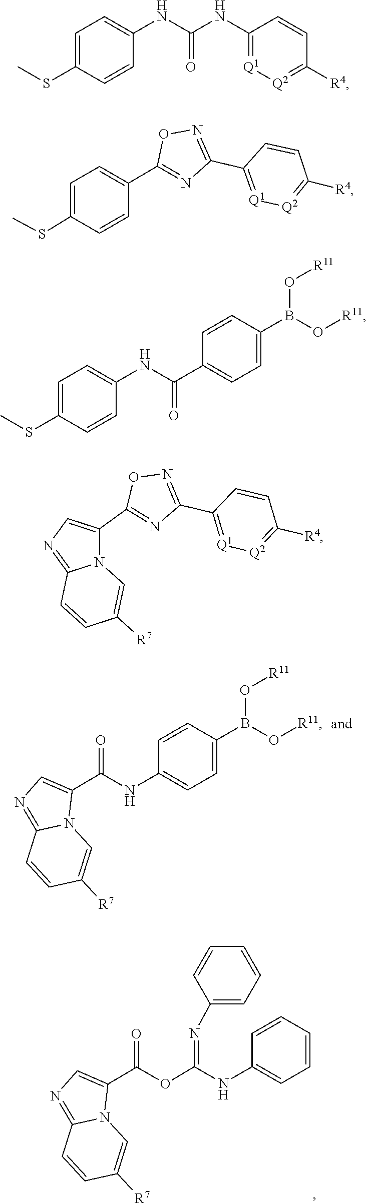

or a pharmaceutically acceptable salt thereof.

In a further aspect, the compound has a structure represented by a formula selected from:

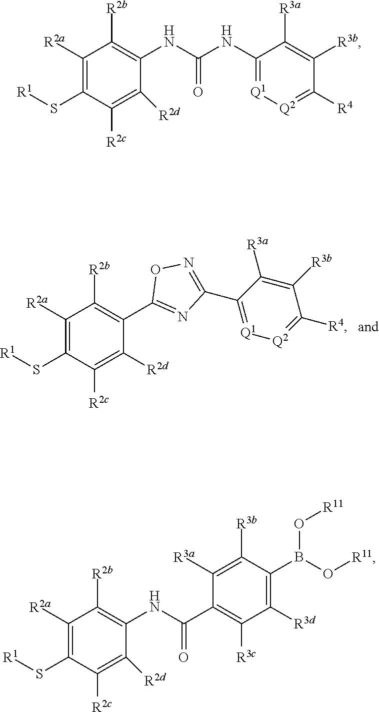

or a pharmaceutically acceptable salt thereof.

In a further aspect, the compound has a structure represented by a formula:

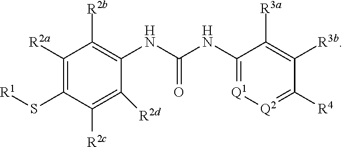

In a further aspect, the compound has a structure represented by a formula:

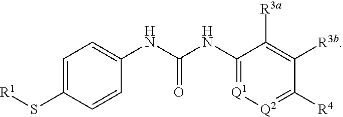

In a further aspect, the compound has a structure represented by a formula:

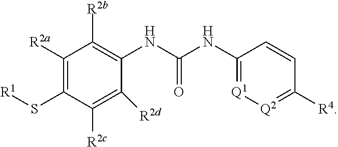

In a further aspect, the compound has a structure represented by a formula:

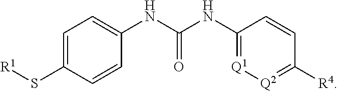

In a further aspect, the compound has a structure represented by a formula:

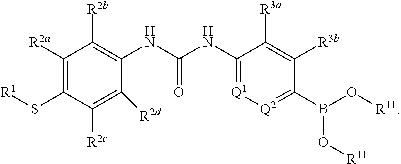

In a further aspect, the compound the compound is selected from:

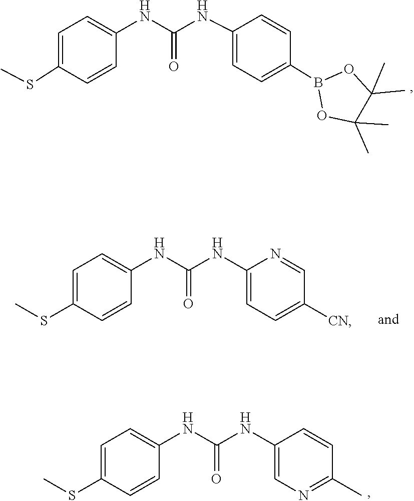

or a pharmaceutically acceptable salt thereof.

In a further aspect, the compound has a structure represented by a formula:

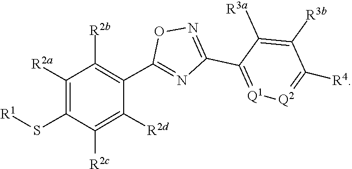

In a further aspect, the compound has a structure represented by a formula:

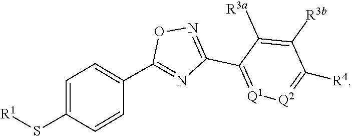

In a further aspect, the compound has a structure represented by a formula:

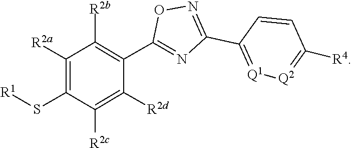

In a further aspect, the compound has a structure represented by a formula:

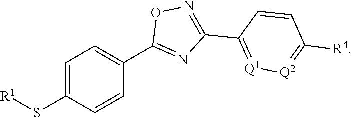

In a further aspect, the compound has a structure represented by a formula:

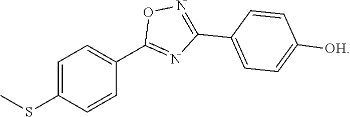 [SMILES: CSc1ccc(-c2nc(-c3ccc(O)cc3)no2)cc1]

In a further aspect, the compound has a structure represented by a formula:

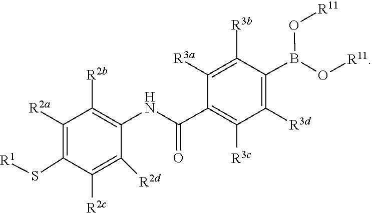

In a further aspect, the compound has a structure represented by a formula:

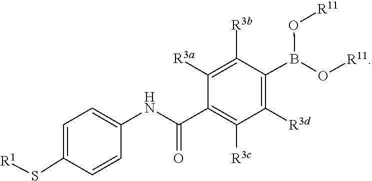

In a further aspect, the compound has a structure represented by a formula:

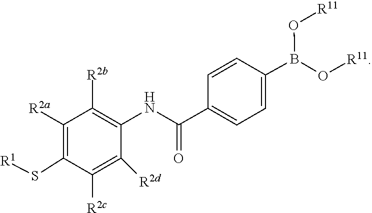

In a further aspect, the compound has a structure represented by a formula:

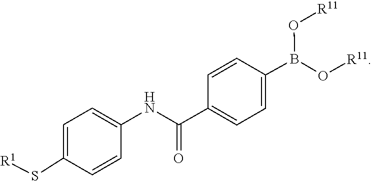

In a further aspect, the compound the compound is selected from:

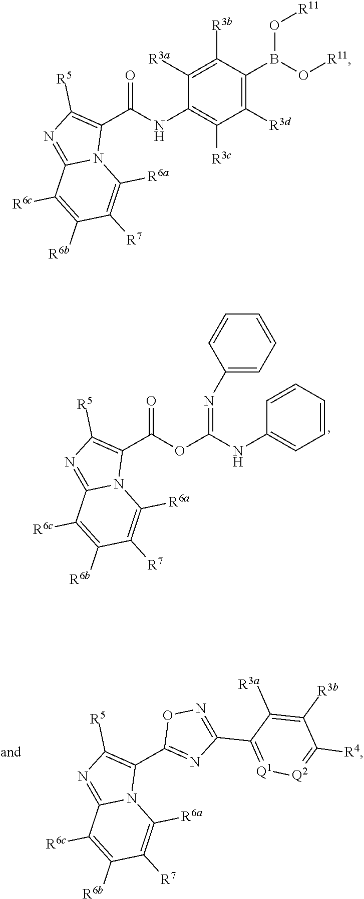

or a pharmaceutically acceptable salt thereof.

In a further aspect, the compound has a structure represented by a formula:

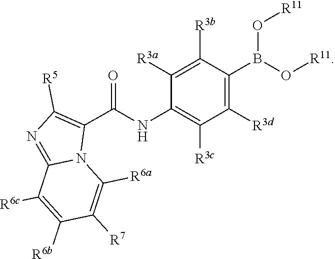

In a further aspect, the compound has a structure represented by a formula:

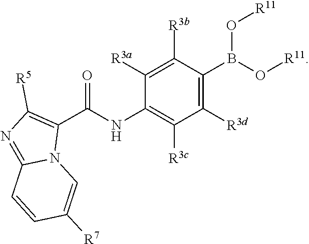

In a further aspect, the compound has a structure represented by a formula:

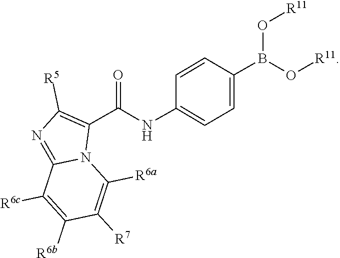

In a further aspect, the compound has a structure represented by a formula:

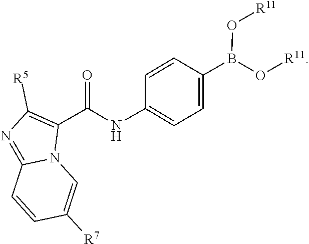

In a further aspect, the compound has a structure represented by a formula:

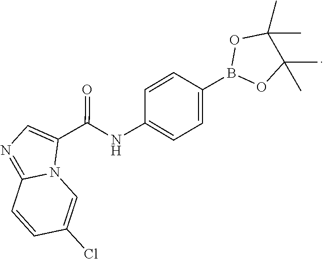 [SMILES: CC1(C)OB(c2ccc(NC(=O)c3cnc4ccc(Cl)cn34)cc2)OC1(C)C]

In a further aspect, the compound has a structure represented by a formula:

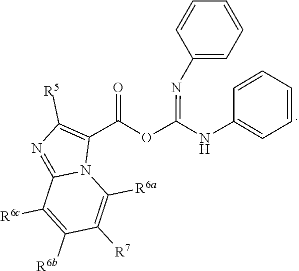

In a further aspect, the compound has a structure represented by a formula:

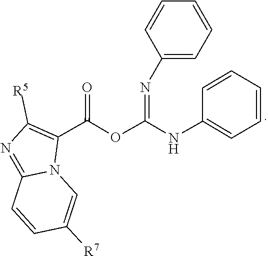

In a further aspect, the compound has a structure represented by a formula:

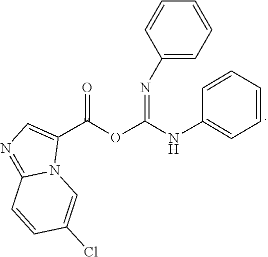 [SMILES: O=C(O/C(=N/c1ccccc1)Nc1ccccc1)c1cnc2ccc(Cl)cn12]

In a further aspect, the compound has a structure represented by a formula:

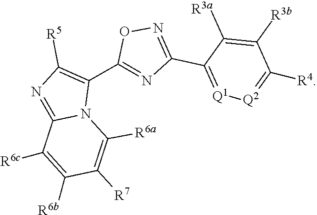

In a further aspect, the compound has a structure represented by a formula:

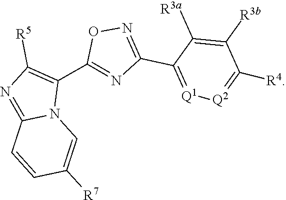

In a further aspect, the compound has a structure represented by a formula:

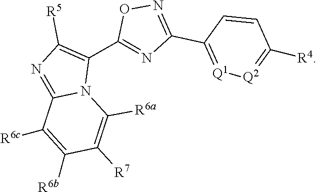

In a further aspect, the compound has a structure represented by a formula:

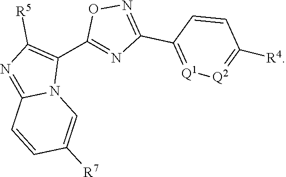

In a further aspect, the compound has a structure represented by a formula selected from:

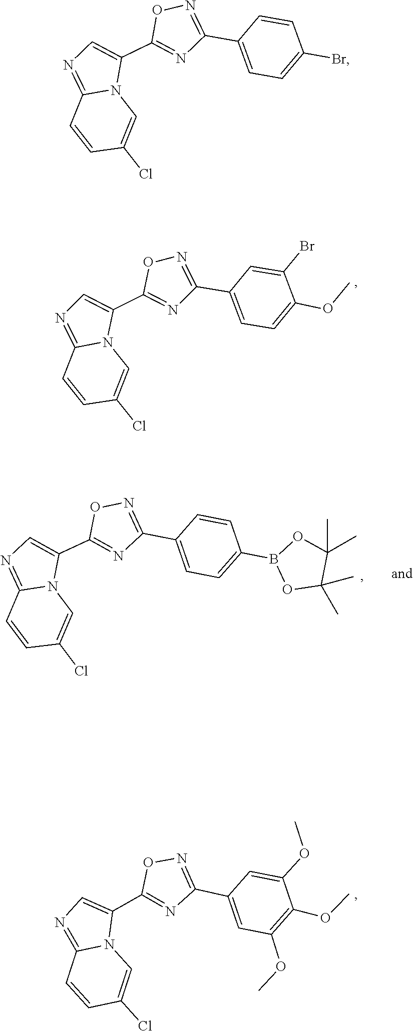 [SMILES: Clc1ccc2ncc(-c3nc(-c4ccc(Br)cc4)no3)n2c1 ; COc1ccc(-c2noc(-c3cnc4ccc(Cl)cn34)n2)cc1Br ; CC1(C)OB(c2ccc(-c3noc(-c4cnc5ccc(Cl)cn45)n3)cc2)OC1(C)C ; COc1cc(-c2noc(-c3cnc4ccc(Cl)cn34)n2)cc(OC)c1OC]

a. Q¹ and Q² Groups

In one aspect, each of Q¹ and Q², when present, is independently selected from N and CR¹⁰. In a further aspect, one of Q¹ and Q², when present, is N, and one of Q¹ and Q², when present, is CR¹⁰. In a still further aspect, each of Q¹ and Q², when present, is N. In yet a further aspect, each of Q¹ and Q², when present, is CR¹⁰. In an even further aspect, each of Q¹ and Q², when present, is CH.

In a further aspect, Q¹, when present, is N, and Q², when present, is CR¹⁰. In a still further aspect, Q¹, when present, is N, and Q², when present, is CH.

In a further aspect, Q¹, when present, is CR¹⁰, and Q², when present, is N. In a still further aspect, Q¹, when present, is CH, and Q², when present, is N.

b. Z¹ and Z² Groups

In one aspect, Z¹, when present, is selected from N and CR²b. In a further aspect, Z¹, when present, is N. In a still further aspect, Z¹, when present, is CR²b.

In one aspect, Z², when present, is selected from N and CR²c. In a further aspect, Z², when present, is N. In a still further aspect, Z², when present, is CR²c.

In various aspects, Z¹, when present, is N, and Z², when present, is N. In a further aspect, Z¹, when present, is CR²b, and Z², when present, is N. In a still further aspect, Z¹, when present, is N, and Z², when present, is CR^{2c}. In yet a further aspect, Z¹, when present, is CR²b, and Z², when present, is CR^{2c}.

c. R¹ Groups

In one aspect, R¹, when present, is C1-C4 alkyl. In a further aspect, R¹, when present, is selected from methyl, ethyl, n-propyl, and isopropyl. In a still further aspect, R¹, when present, is selected from methyl and ethyl. In yet a further aspect, R¹, when present, is ethyl. In an even further aspect, R¹, when present, is methyl.

d. R^{2a}, R^{2b}, R^{2c}, R^{2d}, R^{3a}, and R^{3b} Groups

In one aspect, each of R^{2a}, R^{2b}, R^{2c}, R^{2d}, R^{3a}, and R^{3b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In yet a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, each of R^{2a}, R^{2b}, R^{2c}, R^{2d}, R^{3a}, and R^{3b}, when present, is independently selected from hydrogen, halogen, —CN, —NH₂, —OH, —NO₂, C1-C4 alkyl, and C2-C4 alkenyl. In a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, methyl, ethyl, n-propyl, isopropyl, ethenyl, n-propenyl, and isopropenyl. In a still further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, methyl, ethyl, and ethenyl. In yet a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, and methyl.

In various aspects, each of R^{2a}, R^{2b}, R^{2c}, R^{2d}, R^{3a}, and R^{3b}, when present, is independently selected from hydrogen, halogen, —CN, —NH₂, —OH, —NO₂, C1-C4 haloalkyl, and C1-C4 cyanoalkyl. In a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, and —CH(CH₃)CH₂CN. In a still further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, —CH₂F, —CH₂Cl, —CH₂CH₂F, —CH₂CH₂Cl, —CH₂CN, and —CH₂CH₂CN. In yet a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, —CH₂F, —CH₂Cl, and —CH₂CN.

In various aspects, each of R^{2a}, R^{2b}, R^{2c}, R^{2d}, R^{3a}, and R^{3b}, when present, is independently selected from hydrogen, halogen, —CN, —NH₂, —OH, —NO₂, C1-C4 hydroxyalkyl, C1-C4 haloalkoxy, and C1-C4 alkoxy. In a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, and —OCH(CH₃)CH₃. In a still further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, —CH₂OH, —CH₂CH₂OH, —OCF₃, —OCH₂CF₃, —OCH₃, and —OCH₂CH₃. In yet a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, —CH₂OH, —OCF₃, and —OCH₃.

In various aspects, each of R^{2a}, R^{2b}, R^{2c}, R^{2d}, R^{3a}, and R^{3b}, when present, is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In yet a further aspect, each of R^{2a}, R^{2b}, R^{2c}, R^{2d}, R^{3a}, and R^{3b}, when present, is independently selected from hydrogen, —F, —Cl, —CN, —NH₂, —OH, —NO₂, —NHCH₃, —N(CH₃)₂, and —CH₂NH₂.

In a further aspect, each of R^{2a}, R^{2b}, R^{2c}, R^{2d}, R^{3a}, and R^{3b}, when present, is independently selected from hydrogen and C1-C4 alkyl. In a still further aspect, each of R^{2a}, R^{2b}, R^{2c}, R^{2d}, R^{3a}, and R^{3b}, when present, is independently selected from hydrogen, methyl, ethyl, n-propyl, and isopropyl. In yet a further aspect, each of R^{2a}, R^{2b}, R^{2c}, R^{2d}, R^{3a}, and R^{3b}, when present, is independently selected from hydrogen, methyl, and ethyl. In an even further aspect, each of R^{2a}, R^{2b}, R^{2c}, R^{2d}, R^{3a}, and R^{3b}, when present, is independently selected from hydrogen and ethyl. In a still further aspect, each of R^{2a}, R^{2b}, R^{2c}, R^{2d}, R^{3a}, and R^{3b}, when present, is independently selected from hydrogen and methyl.

In a further aspect, each of R^{2a}, R^{2b}, R^{2c}, R^{2d}, R^{3a}, and R^{3b}, when present, is independently selected from hydrogen and halogen. In a still further aspect, each of R^{2a}, R^{2b}, R^{2c}, R^{2d}, R^{3a}, and R^{3b}, when present, is independently selected from hydrogen, —F, —Cl, and —Br. In yet a further aspect, each of R^{2a}, R^{2b}, R^{2c}, R^{2d}, R^{3a}, and R^{3b}, when present, is independently selected from hydrogen, —F, and —Cl. In an even further aspect, each of R^{2a}, R^{2b}, R^{2c}, R^{2d}, R^{3a}, and R^{3b}, when present, is independently selected from hydrogen and —Cl. In a still further aspect, each of R^{2a}, R^{2b}, R^{2c}, R^{2d}, R^{3a}, and R^{3b}, when present, is independently selected from hydrogen and —F.

In a further aspect, each of R^{2a}, R^{2b}, R^{2c}, R^{2d}, R^{3a}, and R^{3b}, when present, is hydrogen.

e. R^{3c} and R^{3d} Groups

In one aspect, each of R^{3c} and R^{3d}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{3c} and R^{3d}, when present, is independently selected from hydrogen, —F, —Cl, —CN, —NH₂, —OH, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, each of R^{3c} and R^{3d}, when present, is independently selected from hydrogen, —F, —Cl, —CN, —NH₂, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In yet a further aspect, each of R^{3c} and R^{3d}, when present, is independently selected from hydrogen, —F, —Cl, —CN, —NH₂, —OH, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, each of R^{3c} and R^{3d}, when present, is independently selected from hydrogen, halogen, —CN, —NH₂, —OH, —NO₂, C1-C4 alkyl, and C2-C4 alkenyl. In a further aspect, each of R^{3c} and R^{3d}, when present, is independently selected from hydrogen, —F, —Cl, —CN, —NH₂, —OH, —NO₂, methyl, ethyl, n-propyl, isopropyl, ethenyl, n-propenyl, and isopropenyl. In a still further aspect, each of R^{3c} and R^{3d}, when present, is independently selected from hydrogen, —F, —Cl, —CN, —NH₂, —OH, —NO₂, methyl, ethyl, and ethenyl. In yet a further aspect, each of R^{3c} and R^{3d}, when present, is independently selected from hydrogen, —F, —Cl, —CN, —NH₂, —OH, —NO₂, and methyl.

In various aspects, each of R^{3c} and R^{3d}, when present, is independently selected from hydrogen, halogen, —CN, —NH₂, —OH, —NO₂, C1-C4 haloalkyl, and C1-C4 cyanoalkyl. In a further aspect, each of R^{3c} and R^{3d}, when present, is independently selected from hydrogen, —F, —Cl, —CN, —NH₂, —OH, —NO₂, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, and —CH(CH₃)CH₂CN. In a still further aspect, each of R^{3c} and R^{3d}, when present, is independently selected from hydrogen, —F, —Cl, —CN, —NH₂, —OH, —NO₂, —CH₂F, —CH₂Cl, —CH₂CH₂F, —CH₂CH₂Cl, —CH₂CN, and —CH₂CH₂CN. In yet a further aspect, each of R^{3c} and R^{3d}, when present, is independently selected from hydrogen, —F, —Cl, —CN, —NH₂, —OH, —NO₂, —CH₂F, —CH₂Cl, and —CH₂CN.

In various aspects, each of R^{3c} and R^{3d}, when present, is independently selected from hydrogen, halogen, —CN, —NH₂, —OH, —NO₂, C1-C4 hydroxyalkyl, C1-C4 haloalkoxy, and C1-C4 alkoxy. In a further aspect, each of R^{3c} and R^{3d}, when present, is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, and —OCH(CH₃)CH₃. In a still further aspect, each of R^{3c} and R^{3d}, when present, is independently selected from hydrogen, —F, —Cl, —CN, —NH₂, —OH, —NO₂, —CH₂OH, —CH₂CH₂OH, —OCF₃, —OCH₂CF₃, —OCH₃, and —OCH₂CH₃. In yet a further aspect, each of R^{3c} and R^{3d}, when present, is independently selected from hydrogen, —F, —Cl, —CN, —NH₂, —OH, —NO₂, —CH₂OH, —OCF₃, and —OCH₃.

In various aspects, each of R^{3c} and R^{3d}, when present, is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R^{3c} and R^{3d}, when present, is independently selected from hydrogen, —F, —Cl, —CN, —NH₂, —OH, —NO₂, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, each of R^{3c} and R^{3d}, when present, is independently selected from hydrogen, —F, —Cl, —CN, —NH₂, —OH, —NO₂, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In yet a further aspect, each of R^{3c} and R^{3d}, when present, is independently selected from hydrogen, —F, —Cl, —CN, —NH₂, —OH, —NO₂, —NHCH₃, —N(CH₃)₂, and —CH₂NH₂.

In a further aspect, each of R^{3c} and R^{3d}, when present, is independently selected from hydrogen and C1-C4 alkyl. In a still further aspect, each of R^{3c} and R^{3d}, when present, is independently selected from hydrogen, methyl, ethyl, n-propyl, and isopropyl. In yet a further aspect, each of R^{3c} and R^{3d}, when present, is independently selected from hydrogen, methyl, and ethyl. In an even further aspect, each of R^{3c} and R^{3d}, when present, is independently selected from hydrogen and ethyl. In a still further aspect, each of R^{3c} and R^{3d}, when present, is independently selected from hydrogen and methyl.

In a further aspect, each of R^{3c} and R^{3d}, when present, is independently selected from hydrogen and halogen. In a still further aspect, each of R^{3c} and R^{3d}, when present, is independently selected from hydrogen, —F, —Cl, and —Br. In yet a further aspect, each of R^{3c} and R^{3d}, when present, is independently selected from hydrogen, —F, and —Cl. In an even further aspect, each of R^{3c} and R^{3d}, when present, is independently selected from hydrogen and —Cl. In a still further aspect, each of R^{3c} and R^{3d}, when present, is independently selected from hydrogen and —F.

In a further aspect, each of R^{3c} and R^{3d}, when present, is hydrogen.

f. R⁴ Groups

In one aspect, R⁴, when present, is independently selected from halogen, —CN, —OH, C1-C4 alkyl, C1-C4 alkoxy, —B(OR¹¹)₂, and —B(R¹²)₃. In a further aspect, R⁴, when present, is independently selected from —F, —Cl, —Br, —CN, —OH, methyl, ethyl, n-propyl, isopropyl, —OCH₃, —OCH₂CH₃, —OCH₂CH₂CH₃, —OCH(CH₃)CH₃, —B(OR¹¹)₂, and —B(R¹²)₃. In a still further aspect, R⁴, when present, is independently selected from —F, —Cl, —CN, —OH, methyl, ethyl, —OCH₃, —OCH₂CH₃, —B(OR¹¹)₂, and —B(R¹²)₃. In yet a further aspect, R⁴, when present, is independently selected from —F, —CN, —OH, methyl, —OCH₃, —B(OR¹¹)₂, and —B(R¹²)₃.

In one aspect, R⁴, when present, is independently selected from halogen, —CN, —OH, C1-C4 alkyl, C1-C4 alkoxy, and —B(OR¹¹)₂. In a further aspect, R⁴, when present, is independently selected from —F, —Cl, —Br, —CN, —OH, methyl, ethyl, n-propyl, isopropyl, —OCH₃, —OCH₂CH₃, —OCH₂CH₂CH₃, —OCH(CH₃)CH₃, and —B(OR¹)₂. In a still further aspect, R⁴, when present, is independently selected from —F, —Cl, —CN, —OH, methyl, ethyl, —OCH₃, —OCH₂CH₃, and —B(OR¹)₂. In yet a further aspect, R⁴, when present, is independently selected from —F, —CN, —OH, methyl, —OCH₃, and —B(OR¹¹)₂.

In various aspects, R⁴, when present, is independently selected from halogen, —CN, —OH, and —B(OR¹¹)₂. In a further aspect, R⁴, when present, is independently selected from —F, —Cl, —Br, —CN, —OH, and —B(OR¹)₂. In a still further aspect, R⁴, when present, is independently selected from —F, —Cl, —CN, —OH, and —B(OR¹¹)₂. In yet a further aspect, R⁴, when present, is independently selected from —F, —CN, —OH, and —B(OR¹¹)₂.

In various aspects, R⁴, when present, is independently selected from —CN, —OH, and —B(OR¹¹)₂. In a further aspect, R⁴, when present, is independently selected from —CN and —OH. In a still further aspect, R⁴, when present, is —CN. In yet a further aspect, R⁴, when present, is —OH.

In various aspects, R⁴, when present, is independently selected from halogen and —B(OR¹¹)₂. In a further aspect, R⁴, when present, is independently selected from —F, —Cl, —Br, and —B(OR¹)₂. In a still further aspect, R⁴, when present, is independently selected from —F, —Cl, and —B(OR¹)₂. In yet a further aspect, R⁴, when present, is independently selected from —F and —B(OR¹¹)₂.

In various aspects, R⁴, when present, is independently halogen. In a further aspect, R⁴, when present, is independently selected from —F, —Cl, and —Br. In a still further aspect, R⁴, when present, is independently selected from —F and —Cl. In yet a further aspect, R⁴, when present, is —F.

In various aspects, R⁴, when present, is independently selected from C1-C4 alkyl, C1-C4 alkoxy, and —B(OR¹¹)₂. In a further aspect, R⁴, when present, is independently selected from methyl, ethyl, n-propyl, isopropyl, —OCH₃, —OCH₂CH₃, —OCH₂CH₂CH₃, —OCH(CH₃)CH₃, and —B(OR¹¹)₂. In a still further aspect, R⁴, when present, is independently selected from methyl, ethyl, —OCH₃, —OCH₂CH₃, and —B(OR¹¹)₂. In yet a further aspect, R⁴, when present, is independently selected from methyl, —OCH₃, and —B(OR¹¹)₂.

In various aspects, R⁴, when present, is independently selected from C1-C4 alkyl. In a further aspect, R⁴, when present, is independently selected from methyl, ethyl, n-propyl, and isopropyl. In a still further aspect, R⁴, when present, is independently selected from methyl and ethyl. In yet a further aspect, R⁴, when present, is methyl.

In various aspects, R⁴, when present, is independently selected from C1-C4 alkoxy. In a further aspect, R⁴, when present, is independently selected from—OCH₃, —OCH₂CH₃, —OCH₂CH₂CH₃, and —OCH(CH₃)CH₃. In a still further aspect, R⁴, when present, is independently selected from —OCH₃ and —OCH₂CH₃. In yet a further aspect, R⁴, when present, is —OCH₃.

In various aspects, R⁴, when present, is independently selected from —B(OR¹¹)₂ and —B(R¹²)₃. In a further aspect, R⁴, when present, is —B(OR¹¹)₂. In a still further aspect, R⁴, when present, is —B(OH)₂. In yet a further aspect, R⁴, when present, is —B(R¹²)₃. In an even further aspect, R⁴, when present, is —BF₃.

In a further aspect, R⁴, when present, is a structure:

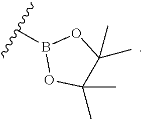 [SMILES: *B1OC(C)(C)C(C)(C)O1]

g. R⁵, R^{6a}, R^{6b}, and R^{6c} Groups

In one aspect, each of R⁵, R^{6a}, R^{6b}, and R^{6c}, when present, is independently selected from hydrogen, halogen, —CN, —NH₂, —OH, —NO₂, C1-C4 alkyl, C2-C4 alkenyl, C₁-C₄ haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R⁵, R^{6a}, R^{6b}, and R^{6c}, when present, is independently selected from hydrogen, —F, —Cl, —CN, —NH₂, —OH, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, each of R⁵, R^{6a}, R^{6b}, and R^{6c}, when present, is independently selected from hydrogen, —F, —Cl, —CN, —NH₂, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In yet a further aspect, each of R⁵, R^{6a}, R^{6b}, and R^{6c}, when present, is independently selected from hydrogen, —F, —Cl, —CN, —NH₂, —OH, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, each of R⁵, R^{6a}, R^{6b}, and R^{6c}, when present, is independently selected from hydrogen, halogen, —CN, —NH₂, —OH, —NO₂, C1-C4 alkyl, and C2-C4 alkenyl. In a further aspect, each of R⁵, R^{6a}, R^{6b}, and R^{6c}, when present, is independently selected from hydrogen, —F, —Cl, —CN, —NH₂, —OH, —NO₂, methyl, ethyl, n-propyl, isopropyl, ethenyl, n-propenyl, and isopropenyl. In a still further aspect, each of R⁵, R^{6a}, R^{6b}, and R^{6c}, when present, is independently selected from hydrogen, —F, —Cl, —CN, —NH₂, —OH, —NO₂, methyl, ethyl, and ethenyl. In yet a further aspect, each of R⁵, R^{6a}, R^{6b}, and R^{6c}, when present, is independently selected from hydrogen, —F, —Cl, —CN, —NH₂, —OH, —NO₂, and methyl.

In various aspects, each of R⁵, R^{6a}, R^{6b}, and R^{6c}, when present, is independently selected from hydrogen, halogen, —CN, —NH₂, —OH, —NO₂, C1-C4 haloalkyl, and C1-C4 cyanoalkyl. In a further aspect, e each of R⁵, R^{6a}, R^{6b}, and R^{6c}, when present, is independently selected from hydrogen, —F, —Cl, —CN, —NH₂, —OH, —NO₂, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, and —CH(CH₃)CH₂CN. In a still further aspect, each of R⁵, R^{6a}, R^{6b}, and R^{6c}, when present, is independently selected from hydrogen, —F, —Cl, —CN, —NH₂, —OH, —NO₂, —CH₂F, —CH₂Cl, —CH₂CH₂F, —CH₂CH₂Cl, —CH₂CN, and —CH₂CH₂CN. In yet a further aspect, each of R⁵, R^{6a}, R^{6b}, and R^{6c}, when present, is independently selected from hydrogen, —F, —Cl, —CN, —NH₂, —OH, —NO₂, —CH₂F, —CH₂Cl, and —CH₂CN.

In various aspects, each of R⁵, R^{6a}, R^{6b}, and R^{6c}, when present, is independently selected from hydrogen, halogen, —CN, —NH₂, —OH, —NO₂, C1-C4 hydroxyalkyl, C1-C4 haloalkoxy, and C1-C4 alkoxy. In a further aspect, each of R⁵, R^{6a}, R^{6b}, and R^{6c}, when present, is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, and —OCH(CH₃)CH₃. In a still further aspect, each of R⁵, R^{6a}, R^{6b}, and R^{6c}, when present, is independently selected from hydrogen, —F, —Cl, —CN, —NH₂, —OH, —NO₂, —CH₂OH, —CH₂CH₂OH, —OCF₃, —OCH₂CF₃, —OCH₃, and —OCH₂CH₃. In yet a further aspect, each of R⁵, R^{6a}, R^{6b}, and R^{6c}, when present, is independently selected from hydrogen, —F, —Cl, —CN, —NH₂, —OH, —NO₂, —CH₂OH, —OCF₃, and —OCH₃.

In various aspects, each of R⁵, R^{6a}, R^{6b}, and R^{6c}, when present, is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R⁵, R^{6a}, R^{6b}, and R^{6c}, when present, is independently selected from hydrogen, —F, —Cl, —CN, —NH₂, —OH, —NO₂, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, each of R⁵, R^{6a}, R^{6b}, and R^{6c}, when present, is independently selected from hydrogen, —F, —Cl, —CN, —NH₂, —OH, —NO₂, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In yet a further aspect, each of R⁵, R^{6a}, R^{6b}, and R^{6c}, when present, is independently selected from hydrogen, —F, —Cl, —CN, —NH₂, —OH, —NO₂, —NHCH₃, —N(CH₃)₂, and —CH₂NH₂.

In a further aspect, each of R⁵, R^{6a}, R^{6b}, and R^{6c}, when present, is independently selected from hydrogen and C1-C4 alkyl. In a still further aspect, each of R⁵, R^{6a}, R^{6b}, and R^{6c}, when present, is independently selected from hydrogen, methyl, ethyl, n-propyl, and isopropyl. In yet a further aspect, each of R⁵, R^{6a}, R^{6b}, and R^{6c}, when present, is independently selected from hydrogen, methyl, and ethyl. In an even further aspect, each of R⁵, R^{6a}, R^{6b}, and R^{6c}, when present, is independently selected from hydrogen and ethyl. In a still further aspect, each of R⁵, R^{6a}, R^{6b}, and R^{6c}, when present, is independently selected from hydrogen and methyl.

In a further aspect, each of R⁵, R^{6a}, R^{6b}, and R^{6c}, when present, is independently selected from hydrogen and halogen. In a still further aspect, each of R⁵, R^{6a}, R^{6b}, and R^{6c}, when present, is independently selected from hydrogen, —F, —Cl, and —Br. In yet a further aspect, each of R⁵, R^{6a}, R^{6b}, and R^{6c}, when present, is independently selected from hydrogen, —F, and —Cl. In an even further aspect, each of R⁵, R^{6a}, R^{6b}, and R^{6c}, when present, is independently selected from hydrogen and —Cl. In a still further aspect, each of R⁵, R^{6a}, R^{6b}, and R^{6c}, when present, is independently selected from hydrogen and —F.

In a further aspect, each of R⁵, R^{6a}, R^{6b}, and R^{6c}, when present, is hydrogen.

h. R⁷ Groups

In one aspect, R⁷, when present, is halogen. In a further aspect, R⁷, when present, is selected from —F, —Cl, and —Br. In a still further aspect, R⁷, when present, is selected from —F, —Cl, and —I. In yet a further aspect, R⁷, when present, is selected from —F and —Cl. In an even further aspect, R⁷, when present, is selected from —F and —Br. In a still further aspect, R⁷, when present, is selected from —Br and —Cl. In yet a further aspect, R⁷, when present, is selected from —F and —I. In an even further aspect, R⁷, when present, is selected from —I and —Cl. In a still further aspect, R⁷, when present, is selected from —I and —Br.

In a further aspect, R⁷, when present, is —Cl. In a still further aspect, R⁷, when present, is —Br. In yet a further aspect, R⁷, when present, is —I. In an even further aspect, R⁷, when present, is —F.

i. R⁸ Groups

In one aspect, R⁸, when present, is selected from —B(OR¹¹)₂ and —B(R¹²)₃. In a further aspect, R⁸, when present, is —B(OR¹¹)₂. In a still further aspect, R⁸, when present, is —B(OH)₂. In yet a further aspect, R⁸, when present, is —B(R¹²)₃. In an even further aspect, R⁸, when present, is —BF₃.

j. R¹⁰ Groups

In one aspect, R¹⁰, when present, is selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, R¹⁰, when present, is selected from hydrogen, —F, —Cl, —CN, —NH₂, —OH, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, R¹⁰, when present, is selected from hydrogen, —F, —Cl, —CN, —NH₂, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In yet a further aspect, R¹⁰, when present, is selected from hydrogen, —F, —Cl, —CN, —NH₂, —OH, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, R¹⁰, when present, is selected from hydrogen, halogen, —CN, —NH₂, —OH, —NO₂, C1-C4 alkyl, and C2-C4 alkenyl. In a further aspect, R¹⁰, when present, is independently selected from hydrogen, —F, —Cl, —CN, —NH₂, —OH, —NO₂, methyl, ethyl, n-propyl, isopropyl, ethenyl, n-propenyl, and isopropenyl. In a still further aspect, R¹⁰, when present, is independently selected from hydrogen, —F, —Cl, —CN, —NH₂, —OH, —NO₂, methyl, ethyl, and ethenyl. In yet a further aspect, R¹⁰, when present, is selected from hydrogen, —F, —Cl, —CN, —NH₂, —OH, —NO₂, and methyl.

In various aspects, R¹⁰, when present, is selected from hydrogen, halogen, —CN, —NH₂, —OH, —NO₂, C1-C4 haloalkyl, and C1-C4 cyanoalkyl. In a further aspect, R¹⁰, when present, is selected from hydrogen, —F, —Cl, —CN, —NH₂, —OH, —NO₂, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, and —CH(CH₃)CH₂CN. In a still further aspect, R¹⁰, when present, is selected from hydrogen, —F, —Cl, —CN, —NH₂, —OH, —NO₂, —CH₂F, —CH₂Cl, —CH₂CH₂F, —CH₂CH₂Cl, —CH₂CN, and —CH₂CH₂CN. In yet a further aspect, R¹⁰, when present, is selected from hydrogen, —F, —Cl, —CN, —NH₂, —OH, —NO₂, —CH₂F, —CH₂Cl, and —CH₂CN.

In various aspects, R¹⁰, when present, is selected from hydrogen, halogen, —CN, —NH₂, —OH, —NO₂, C1-C4 hydroxyalkyl, C1-C4 haloalkoxy, and C1-C4 alkoxy. In a further aspect, R¹⁰, when present, is selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, and —OCH(CH₃)CH₃. In a still further aspect, R¹⁰, when present, is selected from hydrogen, —F, —Cl, —CN, —NH₂, —OH, —NO₂, —CH₂OH, —CH₂CH₂OH, —OCF₃, —OCH₂CF₃, —OCH₃, and —OCH₂CH₃. In yet a further aspect, R¹⁰, when present, is selected from hydrogen, —F, —Cl, —CN, —NH₂, —OH, —NO₂, —CH₂OH, —OCF₃, and —OCH₃.

In various aspects, R¹⁰, when present, is selected from hydrogen, halogen, —CN, —NH₂, —OH, —NO₂, C1-C4 alkylamino, (C1-C4)(C1-C4) dialkylamino, and C1-C4 aminoalkyl. In a further aspect, R¹⁰, when present, is selected from hydrogen, —F, —Cl, —CN, —NH₂, —OH, —NO₂, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, R¹⁰, when present, is selected from hydrogen, —F, —Cl, —CN, —NH₂, —OH, —NO₂, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In yet a further aspect, R¹⁰, when present, is selected from hydrogen, —F, —Cl, —CN, —NH₂, —OH, —NO₂, —NHCH₃, —N(CH₃)₂, and —CH₂NH₂.

In a further aspect, R¹⁰, when present, is selected from hydrogen and C1-C4 alkyl. In a still further aspect, R¹⁰, when present, is selected from hydrogen, methyl, ethyl, n-propyl, and isopropyl. In yet a further aspect, R¹⁰, when present, is selected from hydrogen, methyl, and ethyl. In an even further aspect, R¹⁰, when present, is selected from hydrogen and ethyl. In a still further aspect, R¹⁰, when present, is selected from hydrogen and methyl.

In a further aspect, R¹⁰, when present, is selected from hydrogen and halogen. In a still further aspect, R¹⁰, when present, is selected from hydrogen, —F, —Cl, and —Br. In yet a further aspect, R¹⁰, when present, is selected from hydrogen, —F, and —Cl. In an even further aspect, R¹⁰, when present, is selected from hydrogen and —Cl. In a still further aspect, R¹⁰, when present, is selected from hydrogen and —F.

In a further aspect, R¹⁰, when present, is hydrogen.

k. R¹¹ Groups

In one aspect, each occurrence of R¹¹, when present, is independently selected from hydrogen and C1-C8 alkyl, or each occurrence of R¹¹, when present, is covalently bonded together, and, together with the intermediate atoms, comprise a C6 bicyclic heterocycle or a C2-C3 heterocycloalkyl, and is substituted with 0, 1, 2, 3, or 4 C1-C4 alkyl groups.

In a further aspect, each occurrence of R¹¹, when present, is independently selected from hydrogen and C1-C8 alkyl. In a still further aspect, each occurrence of R¹¹, when present, is independently selected from hydrogen and C1-C4 alkyl. In yet a further aspect, each occurrence of R¹¹, when present, is independently selected from hydrogen, methyl, ethyl, n-propyl, and isopropyl. In an even further aspect, each occurrence of R¹¹, when present, is independently selected from hydrogen, methyl, and ethyl. In a still further aspect, each occurrence of R¹¹, when present, is independently selected from hydrogen and ethyl. In yet a further aspect, each occurrence of R¹¹, when present, is independently selected from hydrogen and methyl.

In a further aspect, each occurrence of R¹¹, when present, is C1-C8 alkyl. In a still further aspect, each occurrence of R¹¹, when present, is C1-C4 alkyl. In yet a further aspect, each occurrence of R¹¹, when present, is selected from methyl, ethyl, n-propyl, and isopropyl. In an even further aspect, each occurrence of R¹¹, when present, is selected from methyl and ethyl. In a still further aspect, each occurrence of R¹¹, when present, is ethyl. In yet a further aspect, each occurrence of R¹¹, when present, is methyl.

In a further aspect, each occurrence of R¹¹, when present, is hydrogen.

In a further aspect, each occurrence of R¹¹, when present, is covalently bonded together, and, together with the intermediate atoms, comprise a C6 bicyclic heterocycle or a C2-C3 heterocycloalkyl, and is substituted with 0, 1, 2, 3, or 4 C1-C4 alkyl groups. In a still further aspect, R¹¹, when present, is covalently bonded together, and, together with the intermediate atoms, comprise a C6 bicyclic heterocycle or a C2-C3 heterocycloalkyl, and is substituted with 0, 1, 2, or 3 C1-C4 alkyl groups. In yet a further aspect, R¹¹, when present, is covalently bonded together, and, together with the intermediate atoms, comprise a C6 bicyclic heterocycle or a C2-C3 heterocycloalkyl, and is substituted with 0, 1, or 2 C1-C4 alkyl groups. In an even further aspect, R¹¹, when present, is covalently bonded together, and, together with the intermediate atoms, comprise a C6 bicyclic heterocycle or a C2-C3 heterocycloalkyl, and is substituted with 0 or 1 C1-C4 alkyl groups. In a still further aspect, R¹¹, when present, is covalently bonded together, and, together with the intermediate atoms, comprise a C6 bicyclic heterocycle or a C2-C3 heterocycloalkyl, and is monosubstituted with a C1-C4 alkyl group. In yet a further aspect, R¹¹, when present, is covalently bonded together, and, together with the intermediate atoms, comprise a C6 bicyclic heterocycle or a C2-C3 heterocycloalkyl, and is unsubstituted.

In a further aspect, each occurrence of R¹¹, when present, is covalently bonded together, and, together with the intermediate atoms, comprise a C6 bicyclic heterocycle substituted with 0, 1, 2, 3, or 4 C1-C4 alkyl groups. In a still further aspect, R¹¹, when present, is covalently bonded together, and, together with the intermediate atoms, comprise a C6 bicyclic heterocycle substituted with 0, 1, 2, or 3 C1-C4 alkyl groups. In yet a further aspect, R¹¹, when present, is covalently bonded together, and, together with the intermediate atoms, comprise a C6 bicyclic heterocycle substituted with 0, 1, or 2 C1-C4 alkyl groups. In an even further aspect, R¹¹, when present, is covalently bonded together, and, together with the intermediate atoms, comprise a C6 bicyclic heterocycle substituted with 0 or 1 C1-C4 alkyl groups. In a still further aspect, R¹¹, when present, is covalently bonded together, and, together with the intermediate atoms, comprise a C6 bicyclic heterocycle monosubstituted with a C1-C4 alkyl group. In yet a further aspect, R¹¹, when present, is covalently bonded together, and, together with the intermediate atoms, comprise an unsubstituted C6 bicyclic heterocycle.

In a further aspect, R¹¹, when present, is covalently bonded together, and, together with the intermediate atoms, comprise a C6 bicyclic heterocycle substituted with 4 C1-C4 alkyl groups. In a still further aspect, R¹¹, when present, is covalently bonded together, and, together with the intermediate atoms, comprise a C6 bicyclic heterocycle substituted with 4 groups selected from methyl, ethyl, n-propyl, and isopropyl. In yet a further aspect, R¹¹, when present, is covalently bonded together, and, together with the intermediate atoms, comprise a C6 bicyclic heterocycle substituted with 4 groups selected from methyl and ethyl. In an even further aspect, R¹¹, when present, is covalently bonded together, and, together with the intermediate atoms, comprise a C6 bicyclic heterocycle substituted with 4 methyl groups.

In a further aspect, each occurrence of R¹¹, when present, is covalently bonded together, and, together with the intermediate atoms, comprise a C2-C3 heterocycloalkyl substituted with 0, 1, 2, 3, or 4 C1-C4 alkyl groups. In a still further aspect, R¹¹, when present, is covalently bonded together, and, together with the intermediate atoms, comprise a C2-C3 heterocycloalkyl substituted with 0, 1, 2, or 3 C1-C4 alkyl groups. In yet a further aspect, R¹¹, when present, is covalently bonded together, and, together with the intermediate atoms, comprise a C2-C3 heterocycloalkyl substituted with 0, 1, or 2 C1-C4 alkyl groups. In an even further aspect, R¹¹, when present, is covalently bonded together, and, together with the intermediate atoms, comprise a C2-C3 heterocycloalkyl substituted with 0 or 1 C1-C4 alkyl groups. In a still further aspect, R¹¹, when present, is covalently bonded together, and, together with the intermediate atoms, comprise a C2-C3 heterocycloalkyl monosubstituted with a C1-C4 alkyl group. In yet a further aspect, R¹¹, when present, is covalently bonded together, and, together with the intermediate atoms, comprise an unsubstituted C2-C3 heterocycloalkyl.

In a further aspect, R¹¹, when present, is covalently bonded together, and, together with the intermediate atoms, comprise a C2-C3 heterocycloalkyl substituted with 4 C1-C4 alkyl groups. In a still further aspect, R¹¹, when present, is covalently bonded together, and, together with the intermediate atoms, comprise a C2-C3 heterocycloalkyl substituted with 4 groups selected from methyl, ethyl, n-propyl, and isopropyl. In yet a further aspect, R¹¹, when present, is covalently bonded together, and, together with the intermediate atoms, comprise a C2-C3 heterocycloalkyl substituted with 4 groups selected from methyl and ethyl. In an even further aspect, R¹¹, when present, is covalently bonded together, and, together with the intermediate atoms, comprise a C2-C3 heterocycloalkyl substituted with 4 methyl groups.

l. R¹² Groups

In one aspect, each occurrence of R¹², when present, is independently halogen. In a further aspect, each occurrence of R¹², when present, is independently selected from —F, —Cl, and —I. In a still further aspect, each occurrence of R¹², when present, is independently selected from —F and —Cl. In yet a further aspect, each occurrence of R¹², when present, is —Cl. In an even further aspect, each occurrence of R¹², when present, is —F.

### 2. Example Compounds

In one aspect, a compound can be present as one or more of the following structures:

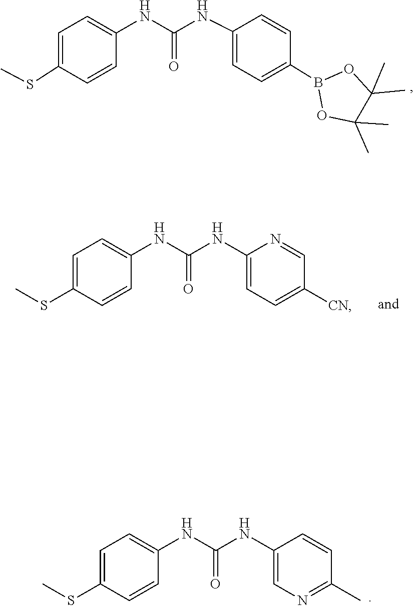

or a pharmaceutically acceptable salt thereof.

In one aspect, a compound can be present as the following structure:

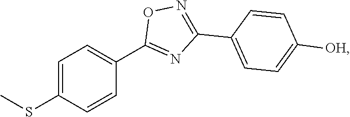 [SMILES: CSc1ccc(-c2nc(-c3ccc(O)cc3)no2)cc1]

or a pharmaceutically acceptable salt thereof.

In one aspect, a compound can be present as the following structure:

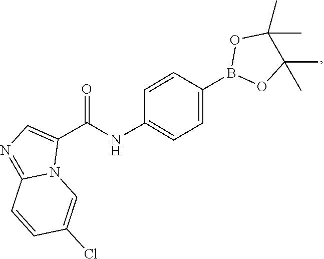 [SMILES: CC1(C)OB(c2ccc(NC(=O)c3cnc4ccc(Cl)cn34)cc2)OC1(C)C]

or a pharmaceutically acceptable salt thereof.

In one aspect, a compound can be present as the following structure:

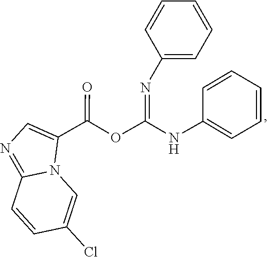 [SMILES: O=C(O/C(=N/c1ccccc1)Nc1ccccc1)c1cnc2ccc(Cl)cn12]

or a pharmaceutically acceptable salt thereof.

In one aspect, a compound can be present as one or more of the following structures:

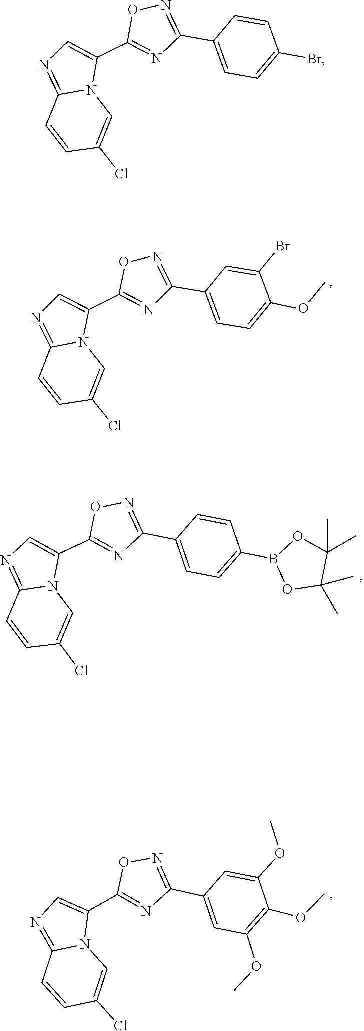 [SMILES: Clc1ccc2ncc(-c3nc(-c4ccc(Br)cc4)no3)n2c1 ; COc1ccc(-c2noc(-c3cnc4ccc(Cl)cn34)n2)cc1Br ; CC1(C)OB(c2ccc(-c3noc(-c4cnc5ccc(Cl)cn45)n3)cc2)OC1(C)C ; COc1cc(-c2noc(-c3cnc4ccc(Cl)cn34)n2)cc(OC)c1OC]

or a pharmaceutically acceptable salt thereof.

In one aspect, a compound can be present as one or more of the following structures:

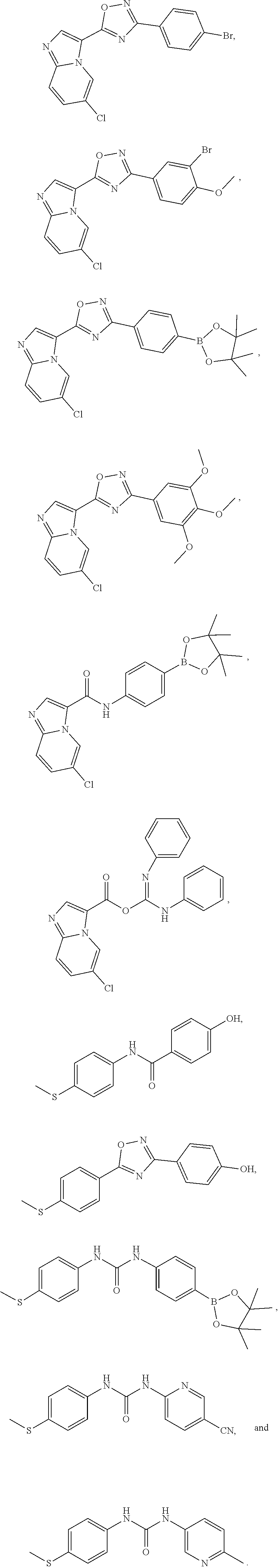

or a pharmaceutically acceptable salt thereof. In one aspect, a compound can be present as one or more of the following structures:

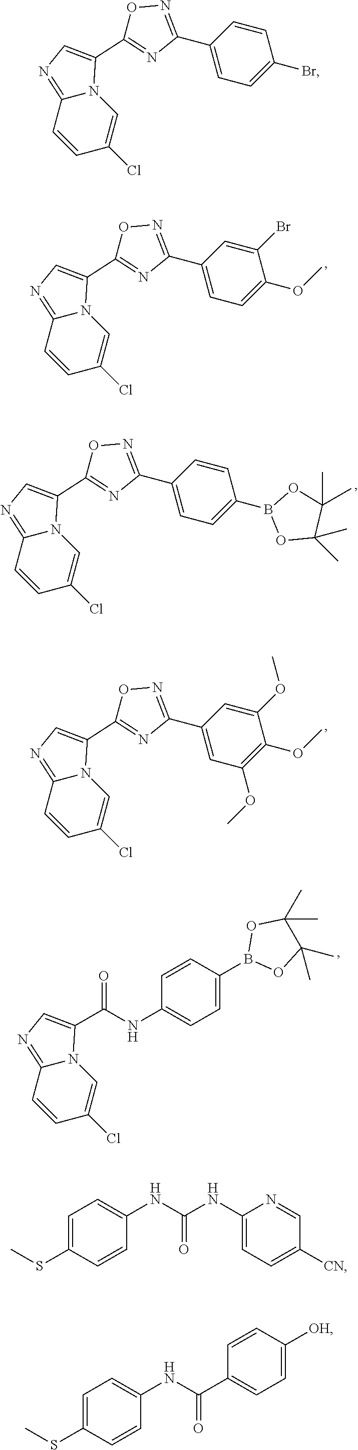

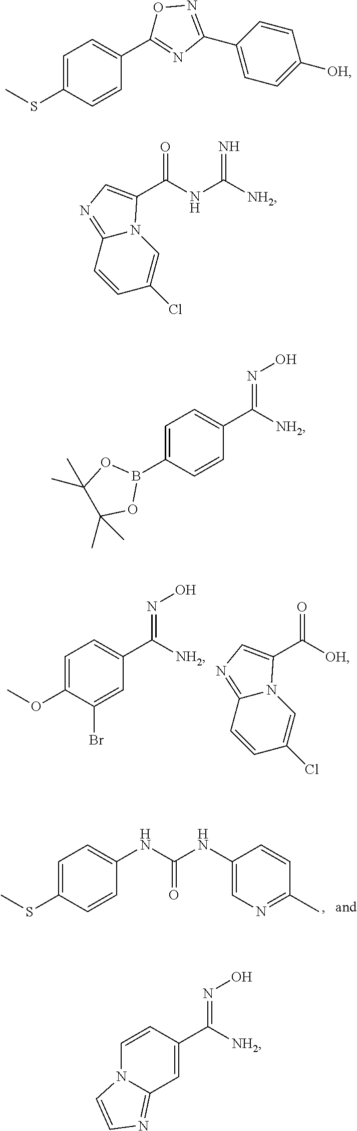

or a pharmaceutically acceptable salt thereof.

### C. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-III, as described and exemplified below. The following examples are provided so that the invention might be more fully understood, are illustrative only, and should not be construed as limiting.

### 1. Route I

In one aspect, substituted small molecule KLF15 agonists can be prepared as shown below.

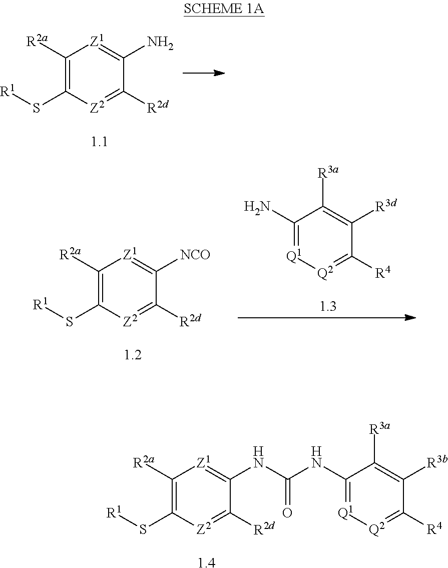

Compounds are represented in generic form, with substituents as noted in compound descriptions elsewhere herein. A more specific example is set forth below.

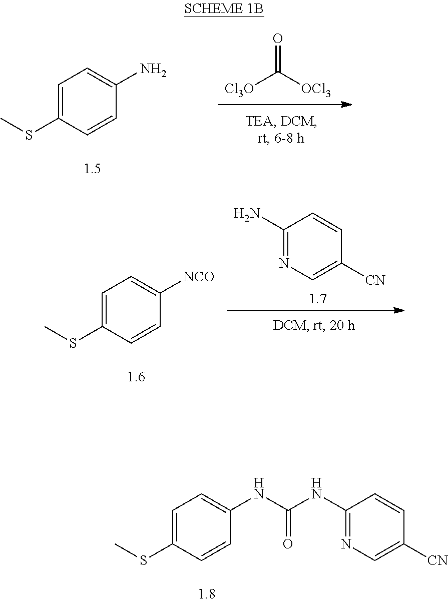

In one aspect, compounds of type 1.8 and similar compounds can be prepared according to reaction Scheme 1B above. Thus, compounds of type 1.6 can be prepared by reacting an appropriate aryl amine, e.g., 1.5 as shown above, with an appropriate electrophilic compound, e.g., 2λ³-trichloran-2-yl trichloro-λ⁴-oxidanecarboxylate as shown above. Appropriate amines and appropriate electrophilic compounds are commercially available or prepared by methods known to one skilled in the art. The reaction is carried out in the presence of an appropriate base, e.g., triethylamine, in an appropriate solvent, e.g., dichloromethane, for an appropriate period of time, e.g., 6 to 8 hours. Compounds of type 1.8 can be prepared by coupling an appropriate isothiocyanate, e.g., 1.6 as shown above, and an appropriate amine, e.g., 1.7 as shown above. The coupling reaction is carried out in the presence of an appropriate solvent, e.g., dichloromethane, for an appropriate period of time, e.g., 20 hours. As can be appreciated by one skilled in the art, the above reaction provides an example of a generalized approach wherein compounds similar in structure to the specific reactants above (compounds similar to compounds of type 1.1, 1.2, and 1.3) can be substituted in the reaction to provide substituted small molecule KLF15 agonists similar to Formula 1.4.

### 2. Route II

In one aspect, substituted small molecule KLF15 agonists can be prepared as shown below.

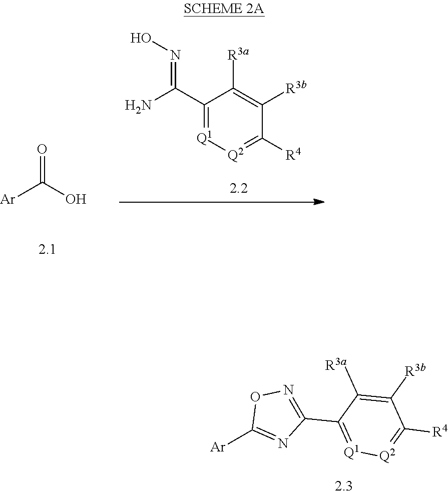

Compounds are represented in generic form, where Ar is

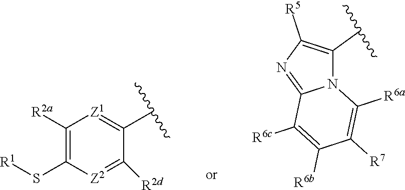

and with other substituents as noted in compound descriptions elsewhere herein. A more specific example is set forth below.

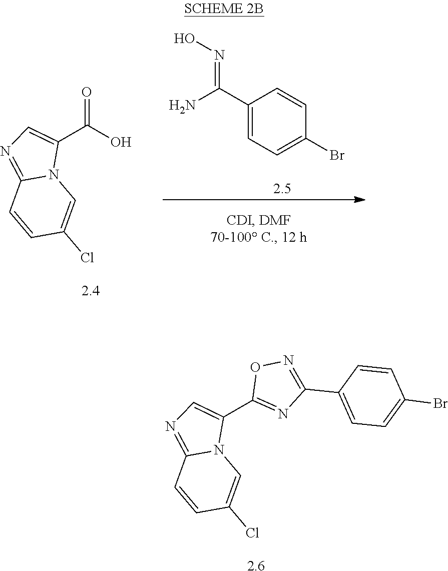

In one aspect, compounds of type 2.6 and similar compounds can be prepared according to reaction Scheme 2B above. Thus, compounds of type 2.6 can be prepared by coupling an appropriate carboxylic acid, e.g., 2.4 as shown above, with an appropriate N-hydroxyimine, e.g., 2.5 as shown above. Appropriate carboxylic acids and appropriate N-hydroxyimines are commercially available or prepared by methods known to one skilled in the art. The coupling reaction is carried out in the presence of an appropriate coupling agent, e.g., 1′-carbonyldiimidazole (CDI), in an appropriate solvent, e.g., dimethylformamide, at an appropriate temperature, e.g., 70 to 100° C., for an appropriate period of time, e.g., 12 hours. As can be appreciated by one skilled in the art, the above reaction provides an example of a generalized approach wherein compounds similar in structure to the specific reactants above (compounds similar to compounds of type 2.1 and 2.2) can be substituted in the reaction to provide substituted small molecule KLF15 agonists similar to Formula 2.3.

### 3. Route III

In one aspect, substituted small molecule KLF15 agonists can be prepared as shown below.

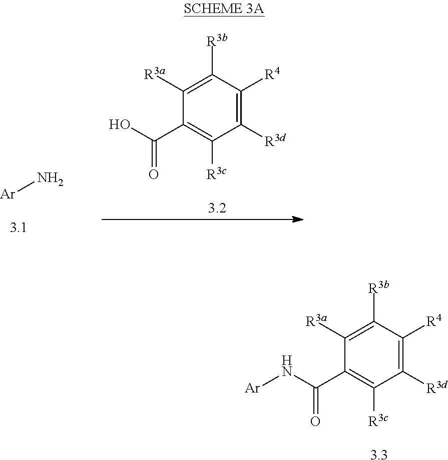

Compounds are represented in generic form, where Ar is

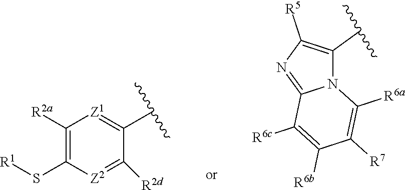

and with other substituents as noted in compound descriptions elsewhere herein. A more specific example is set forth below.

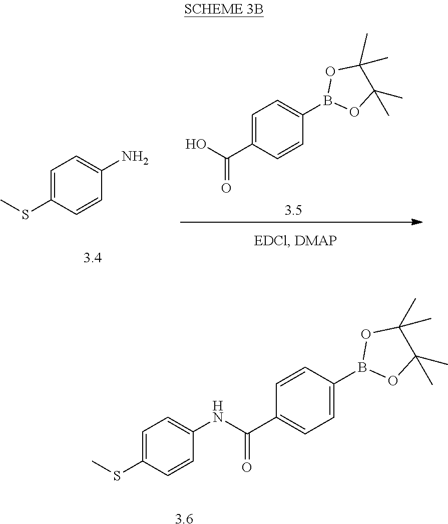

In one aspect, compounds of type 3.6 and similar compounds can be prepared according to reaction Scheme 3B above. Thus, compounds of type 3.6 can be prepared by coupling an appropriate amine, e.g., 3.4 as shown above, with an appropriate carboxylic acid, e.g., 3.5 as shown above. Appropriate amines and appropriate carboxylic acids are commercially available or prepared by methods known to one skilled in the art. The coupling reaction is carried out in the presence of an appropriate coupling agent, e.g., 1-ethyl-3-(3-dimethylaminopropyl)carbodiimide (EDCI), and an appropriate activating agent, e.g., 4-dimethylaminopyridine (DMAP). As can be appreciated by one skilled in the art, the above reaction provides an example of a generalized approach wherein compounds similar in structure to the specific reactants above (compounds similar to compounds of type 3.1 and 3.2) can be substituted in the reaction to provide substituted small molecule KLF15 agonists similar to Formula 3.3.

### D. Additional Methods of Using the Compounds

The compounds and pharmaceutical compositions of the invention are useful in treating or controlling disorders associated with overactivation of IKKβ such as, for example, cancer, arthritis, a cardiometabolic disease, and chronic obstructive pulmonary disease (COPD). To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a disorder associated with overactivation of IKKβ such as, for example, cancer, arthritis, a cardiometabolic disease, and chronic obstructive pulmonary disease (COPD).

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disorder associated with overactivation of IKKβ such as, for example, cancer, arthritis, a cardiometabolic disease, and chronic obstructive pulmonary disease (COPD).

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 1. Use of Compounds

In one aspect, the invention relates to the use of a disclosed compound or a product of a disclosed method. In a further aspect, a use relates to the manufacture of a medicament for the treatment of a disorder associated with KLF15 signaling dysfunction, as further described herein, in a subject.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of disorder associated with overactivation of IKKβ in a subject. In one aspect, the use is characterized in that the subject is a human. In one aspect, the use is characterized in that the disorder is selected from cancer, arthritis, a cardiometabolic disease, and chronic obstructive pulmonary disease (COPD).

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a disorder associated with overactivation of IKKβ in a mammal. In a further aspect, the disorder is selected from cancer (e.g., a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, plasma cell neoplasm (myeloma)), arthritis (e.g., osteoarthritis, psoriatic arthritis, rheumatoid arthritis, juvenile idiopathic arthritis), a cardiometabolic disease (e.g., heart attack, stroke, diabetes, insulin resistance, non-alcoholic fatty liver disease), and chronic obstructive pulmonary disease (COPD).

### 2. Manufacture of a Medicament

In one aspect, the invention relates to a method for the manufacture of a medicament for treating a disorder associated with overactivation of IKKβ in a subject in need thereof, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the inhibition of a viral infection. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### E. Examples

Here, several key conceptually and technically innovative strategies for optimization of lead KLF15 agonists are disclosed: (1) To date, utilization of small molecules to induce the expression of a pro-differentiation transcription factor in kidney disease is conceptually innovative. (2) Although combinatorial therapy to reduce the cumulative GC dose is not new, incorporating this strategy during early pre-IND studies by combining the use of KLF15 agonists and GCs is innovative in maximizing the effectiveness of therapy and optimizing lead KLF15 agonists. (3) The use of human podocyte-based high-throughput screening (HTS) to identify novel small molecules KLF15 agonists is innovative and has not been previously described in glomerular disease. (4) An innovative lead-optimization strategy involving iterative medicinal chemistry in combination with Limited Rational Design (LRD) approach is used to identify and optimize novel KLF15 analogues that will be selective in the low nanomolar (<100 nM) for primary glomerulopathies in a cost-effective and less time-consuming manner. (5) An innovative strategy to augment in vivo PK profiling studies using KLF15 analogues labelled with radiotracer and conduct PET imaging is proposed. (6) Novel assays to assess IL-17RA activity-homogenous ELISA-like solid phase binding and surface plasmon resonance assays are utilized to optimize selectivity of KLF15 agonists. (7) Human kidney organoids are utilized with single-cell RNA-seq to enhance the rigor of the preclinical models for optimization of lead KLF15 agonists. (8) A preliminary SAR was conducted of K-7 and used to identify novel KLF15 analogues.

Without wishing to be bound by theory, the following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Materials and Methods

### A. High-Throughput Drug Screening in Human Podocytes

Methods for immortalized human podocyte cultivation, immortalization, and differentiation were based on previously described protocol (M. A. Saleem et al., (2002) J Am Soc Nephrol 13, 630-638). Briefly, these podocytes proliferate under permissive conditions (gamma interferon at 33° C.), but differentiate under nonpermissive conditions (37° C.). Podocytes in 37° C. for 14 days are noted to be differentiated (M. A. Saleem et al., (2002) J Am Soc Nephrol 13, 630-638).

To generate the human KLF15 reporter assay, a 3636 bp fragment of the human KLF15 promoter upstream of the ATG start codon was amplified by PCR on the RP11 71E19 BAC clone (Resgen Invitrogen). The amplified fragment contains MluI and PmeI RE sites and was subcloned into the MluI and PmeI cloning site of the pEZX-FR03 Firefly luciferase (FLuc) and Renilla luciferase (Rluc) reporter cloning vector (GeneCopoeia) to generate pEZX-FR03hKLF15p. Following verification of the sequence by restriction and μsequencing, the pEZX-FR03hKLF15p construct (which contains a puromycin resistance cassette) was transfected into human podocytes followed by selection in puromycin-containing media for 2 weeks. Surviving podocytes clones were pooled together for further analysis.

KLF15 reporter human podocytes were proliferated under permissive conditions (33° C.) in RPMI 1640 with 5% fetal bovine serum and 1% penicillin/streptomycin supplemented with 1 μg/mL puromycin, and shifted to 37° C. to induce differentiation according to published protocols (S. K. Mallipattu et al., (2017) J Am Soc Nephrol 28, 166-184, S. K. Mallipattu et al., (2012). J Biol Chem 287, 19122-19135). After 7 days at 37° C., cells were trypsinized, counted, and transferred to 96 white plates (Corning 3917), and they were further cultured at 37° C. for another 4 days and subsequently used in an HTS assay (H. W. Lee et al., (2015) J Am Soc Nephrol 26, 2741-2752).

The library of compounds from the National Cancer Institute (Approved Oncology Drugs Set VI; Natural Products Set III; Diversity Set V; and Mechanistic Diversity Set II) were added to a final concentration of 1 μM in 0.5% DMSO in the low FBS cell culture media (0.2% FBS) using liquid handling system PipetMax (Gilson). After 24 hours incubation at 37° C., the Luc-Pair™ Duo-Luciferase HT Assay Kit (GeneCopoeia LF015) were used and firefly and renilla luciferase activity was determined using the SpectraMax M3 (Molecular Devices). Positive controls were all-trans retinoic acid (atRA) (1 μM) and dexamethasone (DEX) (1 μM). Negative controls were DMSO and cell-free media. The data were validated by two independent variables: signal-to-background (S/B) ratio and Z′ factor as previously reported (A. B. Bialkowska, Y. Du, H. Fu, V. W. Yang, (2009) Mol Cancer Ther 8, 563-570).

b. Half Maximal Effective Concentration (EC₅₀)

A serial dilution was initially created to measure the half maximal effective concentration (EC₅₀) with concentrations ranging from 10⁻² to 10⁴ nM. pEZX-FR03hKLF5p human podocytes were subsequently treated with various concentrations for each compound and firefly and renilla luciferase activity were measured. The EC₅₀ was determined using nonlinear regression and then choosing the equation of Sigmoidal function, 4PL method (X is log(concentration); or the equation of log(agonist) vs. response—variable slope).

c. GR-Mutant KLF15 Reporter Assay

A serial dilution was initially created to measure the half maximal effective concentration (EC₅₀) with concentrations ranging from 10⁻² to 10⁴ nM. pEZX-FR03hKLF5p human podocytes were subsequently treated with various concentrations for each compound and firefly and renilla luciferase activity were measured. The EC₅₀ was determined using nonlinear regression and then choosing the equation of Sigmoidal function, 4PL method (X is log(concentration); or the equation of log(agonist) vs. response—variable slope).

d. KLF15 Knockdown

KLF15 knockdown in human podocytes was performed using Genecopoeia lentiviral shRNA system as previously described (S. K. Mallipattu et al., (2017) J Am Soc Nephrol 28, 166-184). In brief, lentiviral particles were produced by transfecting HEK 293T cells with a combination of lentiviral expression plasmid DNA, pCD/NL-BH ΔΔΔ packaging plasmid, and VSV-G-encoding pLTR-G plasmid. Viral supernatants were subsequently supplemented with 8 μg/ml polybrene and incubated with cells for 24 hours. Cells expressing shRNA were selected with puromycin for 2 weeks. mCherry expression and Western blot was performed to confirm KLF15 knockdown, with SC-shRNA as control as reported (S. K. Mallipattu et al., (2017) J Am Soc Nephrol 28, 166-184).

e. Lipopolysaccharide Treatment in Human Podocytes

Differentiated pEZX-FR03hKLF5p human podocytes were treated with 25 μg/ml lipopolysaccharide (LPS) or vehicle and 1 μM KLF15 agonist or vehicle control for 24 hours. Firefly and renilla luciferase assay were measured initially to determine relative KLF15 reporter activity.

f. Adriamycin Treatment in Human Podocytes

Differentiated pEZX-FR03hKLF5p human podocytes were treated with 0.4 μg/ml adriamycin (ADR) or vehicle and 1 μM KLF15 agonist or vehicle control for 24 hours. Firefly and renilla luciferase assay were measured initially to determine relative KLF15 reporter activity.

g. 3-(4, 5-dimethylthiazolyl-2)-2, 5-diphenyltetrazolium bromide (MTS) Assay

To assess cell viability, the CellTiter 96® AQueous One Solution Reagent (Promega) was added to the culture media and plates were incubated for 2 hours at 37° C. with 5% CO₂. Optical density was determined at 490 nm using a 96-well plate reader SpectraMax M3 (Molecular Devices).

h. Actin Stress Fiber Formation

To determine actin stress fiber formation, differentiated human podocytes were treated with 25 μg/ml LPS or vehicle and 1 μM KLF15 agonist or vehicle control for 24 hours. Subsequently, podocytes were fixed and stained for F-actin using Alexa Fluor 647 Phalloidin (Life Technologies). Fixation, permeabilization, and staining with phalloidin were performed as per the manufacturer's protocol. Quantification and classification of changes in actin cytoskeleton are on the basis of previously published methodology (S. K. Mallipattu et al., (2017) J Am Soc Nephrol 28, 166-184). Briefly, phalloidin staining pattern in each cell was classified into the following types: Type A (90% of cell area filled with thick cables), Type B (no thick cables but some cables present), and Type C (no cables visible in the central area of the cell). Unless specified, 100-200 cells were quantified in a blinded manner for each group in three independent experiments.

i. Immunocytochemistry

Differentiated human podocytes were initially washed with phosphate-buffered saline (PBS) and subsequently fixed with 3.7% formaldehyde in the growth medium. Podocytes were washed and permeabilized with 0.25% Triton X-100, and then blocked in 10% normal horse serum (NHS) and incubated with rabbit anti-KLF15 antibody overnight. The next day, cells were washed with PBS and incubated in Donkey anti-Rabbit IgG (H+L) Secondary Antibody, Alexa Fluor™ 568 (Invitrogen) in 10% NHS. Subsequently, cells were washed and incubated with Hoechst (Thermo Fisher Scientific) before mounting.

j. Liquid Chromatography with Tandem Mass Spectrometry (LC-MS-MS)

Mass spectrometry measurements were performed on a TSQ Quantum Access MAX Triple-Stage Quadrupole Mass Spectrometer (Thermo Fisher Scientific) at Stony Brook Medicine Proteomics Center. Samples were introduced to MS via electrospray ionization. The collected spectra were scanned over the mass/charge number (m/z) range of 250-500 atomic mass units (Xcalibur version 2.07). MS spectra were generated by collision-induced dissociation of the metabolite ions at normalized collision energy of approximately 35 eV according to the parent compound. Samples stock solution were 10 mM in DMSO and diluted 1:1000 in acetonitrile (ACN), and then 1:5 in 50% ACN/0.1% formic acid (FA).

k. RNA Sequencing and Enrichment Analysis

RNA sequencing data was processed as previously described (Z. Wang, A. Ma'ayan, (2016) F1000Res 5, 1574). Briefly, sequencing reads were first aligned to the human genome (version hg38) using Spliced Transcripts Alignment to a Reference (STAR 2.4.1c) (A. Dobin et al., (2013) Bioinformatics 29, 15-21). Aligned reads were then quantified to the transcriptome (UCSC hg38 annotation) at the gene level using featureCounts (v1.4.6) (Y. Liao, G. K. Smyth, W. Shi, (2014) Bioinformatics 30, 923-930). Read counts were normalized to count per million (CPM), and differentially expressed genes were identified using BioJupies (A. Lachmann et al., (2018) Nat Commun 9, 1366). Enrichment analyses of the differentially expressed genes were performed with Enrichr (33, 66) with WikiPathways (D. N. Slenter et al., (2018) Nucleic Acids Res 46, D661-d667) and KEGG (M. Kanehisa, S. Goto, (2000) Nucleic Acids Res 28, 27-30) databases. Normalized read counts were used to create matrix visualized heat maps on Morpheus by Broad Institute (https://software.broadinstitute.org/morpheus) with hierarchical clustering, sorted by one minus Pearson's correlation and clustered by rows. Gene set enrichment analysis (GSEA) by Broad Institute and UC San Diego (A. Subramanian et al., (2005) P Natl Acad Sci USA 102, 15545-15550) was conducted using raw RNA-seq counts and used to enrich the gene list for the NF-κB signaling pathway to evaluate differences in gene expression level between different treatment groups. Integrated pathway analysis was conducted using ClusterProfiler (G. C. Yu, et al., (2012) Omics 16, 284-287 (2012)) based on differentially expressed genes between treatment groups and sorted based on gene count and adjusted p-value to identify enriched pathways. Using the TRANSFAC software (V. Matys et al., (2003) Nucleic Acids Res 31, 374-378 (2003), V. Matys et al., (2006) Nucleic Acids Res 34, D108-110 (2006)) the promoters of all human genes in the region from (−2000) to the transcription start site with the KLF15 position weight matrix provided by the TRANSFAC system were scanned. Enrichment analysis was performed using Enrichr and the Fisher's Exact test was used to determine the terms that were overrepresented among the genes with KLF15 binding sites (S. K. Mallipattu et al., (2017) J Am Soc Nephrol 28, 166-184, E. Y. Chen et al., (2013) BMC Bioinformatics 14, 128).

l. In Silico P50/P65 Motif Enrichment

The following R libraries were used to construct the in silico ENCODE ChIP-seq binding of p50 and p65 at the KLF15 promoter-proximal region within the first intron: GenomicRanges, rtracklayer, GenomicFeatures, Gviz, biomaRt, BSgenome.Hsapiens. UCSC.hg38. Gviz was the principal package used to graphically represent the binding of p50 and p65, followed by generation of the ENSEMBL human genome reference (hg38) track which underlies the intronic KLF15 region-of-interest plotting.

m. BT503 Treatment in Proteinuric Murine Models

In the LPS-induced proteinuric model, all 8-week-old FVB/n mice were initially treated with LPS [intraperitoneal (IP), 10 μg/g, Sigma-Aldrich] or sterile normal saline (IP) at 0- and 24-hour time points as previously described (17). BT503 (IP, 1 mg/kg), BT503 (IP, 0.5 mg/kg), DEX (IP, 1 mg/kg), DEX (IP, 10 mg/kg), combination of BT503 (IP, 0.5 mg/kg) and DEX (IP, 0.5 mg/kg), or vehicle (IP, 50% normal saline, 35% PEG300, 5% Tween80, 10% DMSO) were concurrently administered at similar time points. Urine was collected and mice were euthanized 48 hours post-LPS treatment.

In the Nephrotoxic Serum Nephritis (NTS) model, 8-week-old FVB/n mice were initially sensitized with an intraperitoneal injection of 0.5 mg of sheep IgG (IP, Jackson ImmunoResearch) with complete Freund's adjuvant (IP, Millipore Sigma). Five days later, mice were administered 100 μl of NTS (IP, ProbeTex), as previously described (Estrada et al. (2018) JCI Insight 3). All mice were treated with BT503 (IP, 1 mg/kg) or vehicle (IP, 50% normal saline, 35% PEG300, 5% Tween80, 10% DMSO) daily. Urine was collected and mice were euthanized at day 7 or day 14 post-NTS treatment.

In the HIV-1 transgenic model, Tg26 and wildtype FVB/n 8-week-old mice were treated with either BT503 (IP, 1 mg/kg) or vehicle (IP, 50% normal saline, 35% PEG300, 5% Tween80, 10% DMSO) daily. Urine and serum were collected and mice were euthanized 2 weeks after initiation of BT503 treatment.

n. Measurement of Urine Albumin and Creatinine

Urine albumin was quantified by ELISA using a kit from Bethyl Laboratory Inc. Urine creatinine levels were measured in the same samples using the Creatinine Colorimetric Assay Kit (500701; Cayman Chemical) as per manufacturer's protocol.

o. Isolation of Glomeruli from Mice for RNA Extraction

Mouse glomeruli were isolated as described (Mallipattu and He (2016) Am J Physiol Renal Physiol 311, F46-51; Mallipattu et al. (2012) J Biol Chem 287, 19122-19135). Briefly, mice were perfused with PBS containing 2.5 mg/ml iron oxide and 1% BSA. At the end of perfusion, kidneys were removed, decapsulated, minced into 1 mm³ pieces, and digested in PBS containing 1 mg/ml collagenase A. Digested tissue was subsequently passed through a 100 μm cell strainer and collected by centrifugation. The pellet was resuspended in 1 ml PBS and glomeruli were collected using a magnet. The purity of glomeruli was verified under microscopy. Total RNA was isolated from kidney glomeruli of mice using the RNeasy Kit (Qiagen, Germantown, MD).

p. Real-Time PCR

Total RNA from cells was extracted by using TRIzol (Life Technologies). First-strand cDNA was prepared from total RNA (up to 1.25 μg) using the SuperScript™ IV VILO™ Master Mix (Life Technologies), and cDNA was amplified in triplicate using PowerUp™ SYBR™ Green Master Mix on an ABI QuantStudio3 (Applied Biosystems). Primers for human and mouse genes were designed using NCBI Primer-BLAST and validated for efficiency before application (Table 1). Data were normalized to housekeeping genes (GAPDH or ACTB) and presented as a fold increase compared with RNA isolated from the control group using the ΔΔCT method.

TABLE 1

| Gene | | | |
|---|---|---|---|
| Name | Species | Forward primer | Reverse primer |
| KLF15 | Human | GTTGGGTATCTGGGTGATA | TGAGAGTCGGGACTGGAAC |
| | | GGC | AG |
| Syn- | Human | AGCCCAAGGTGACCCCGA | CCCTGTCACGAGGTGCTGG |
| aptopodin | | AT | C |
| WT1 | Human | CAGGCTGCAATAAGAGAT | GAAGTCACACTGGTATGGT |
| | | ATTTTA | TTCT |
| GAPDH | Human | TGTTGCCATCAATGACCCC | CTCCACGACGTACTCAGCG |
| | | TT | |
| ACTB | Human | AGAGCTACGAGCTGCCTGA | AGCACTGTGTTGGCGTACA |
| | | C | G |
| Klf15 | Mouse | AGAGCAGCCACCTCAAGG | TCACACCCGAGTGAGATCG |
| | | CCCA | CCGGT |
| Wt1 | Mouse | GAGAGCCAGCCTACCATCC | GGGTCCTCGTGTTTGAAGG |
| | | | AA |
| Gapdh | Mouse | GCCATCAACGACCCCTTCA | ATGATGACCCGTTTGGCTC |
| | | T | C |
| Actb | Mouse | GTTCCGATGCCCTGAGGCT | CGTCACACTTCATGATGGA |
| | | CTT | ATTGA |

q. Western Blot

Differentiated human podocytes were lysed with a fractionation buffer containing 1× protease and phosphatase inhibitor cocktail (Thermo Fisher Scientific) and separate cytoplasmic and nuclear fractions using a modified procedure described before (72). Cell fractionation lysates from cultured cells were subjected to immunoblot analysis using the antibody for the target protein of interest with mouse anti-GAPDH (Millipore, MAB374) for loading control. Antibodies utilized for target proteins of interest: rabbit anti-KLF15, rabbit anti-p50 (Cell Signaling Tech, 13586S), rabbit anti-p65 (Cell Signaling Tech, 8242S), rabbit anti-IKBα (Cell Signaling Tech, 48125), rabbit anti-IKKα (Cell Signaling Tech, 61294S), rabbit anti-IKKβ (Cell Signaling Tech, 8943S), and rabbit anti-Histone H3 (Cell Signaling Tech, 4620S) antibodies.

r. Immunofluorescence Staining and Quantification

Specimens were initially baked for 60 minutes in a 60° C. oven and then processed as previously described (S. K. Mallipattu et al., (2017) J Am Soc Nephrol 28, 166-184). Briefly formalin-fixed and paraffin embedded sections were deparaffinized, and endogenous peroxidase was inactivated with H₂O₂. All kidney sections from these mice were prepared in identical fashion. Immunofluorescence was performed using rabbit anti-KLF15, mouse anti-WT1 (Santa Cruz, SC-7385), rabbit anti-Synaptopodin (Sigma, SAB3500585), rabbit anti-p65 (Cell Signaling Tech, 8242S) antibodies. After washing, sections were incubated with the appropriate fluorophore-linked secondary antibody (Alexa Fluor 647 Donkey anti-mouse, Alexa Fluor 568 Donkey anti-rabbit antibodies, Life Technologies). After counter staining with Hoechst (Thermo Fisher Scientific), slides were mounted in Prolong Gold mounting media (Thermo Fisher Scientific) and photographed under a Nikon Eclipse Ni-E Fully Motorized Microscope System with a DS-Qi2 digital camera.

Quantification of KLF15 staining in the podocytes was determined by quantifying the intensity of KLF15 staining (optical density) in WT1+Hoechst staining using ImageJ 1.53c software (National Institute of Health, http://imagej.nih.gov/ij). WT1 staining was quantified by counting the number of WT1+ cells per glomerulus area (μm²) Quantification of Synaptopodin staining was performed by measuring % glomerular area staining using ImageJ (S. K. Mallipattu et al., (2017) J Am Soc Nephrol 28, 166-184).

s. Light Microscopy and Histopathological Scoring

Mice were perfused with PBS, and the kidneys were fixed in 10% phosphate buffered formalin overnight and switched to 70% ethanol before processing for histology. Kidney tissue was embedded in paraffin by Stony Brook Medicine Research Histology Core Laboratory (RHCL)—Department of Pathology and 4-μm-thick sections were stained with periodic acid-Schiff (PAS), hematoxylin & eosin (H&E), and masson's trichrome (Sigma-Aldrich). Quantification of % FSGS was determined by renal pathology in a blinded fashion (Huntsman Cancer Institute-University of Utah Health).

t. Transmission Electron Microscopy

Mice were perfused with PBS and then immediately fixed in 2.5% glutaraldehyde for electron microscopy as previously described (S. K. Mallipattu et al., (2017) J Am Soc Nephrol 28, 166-184). Transmission Electron Microscopy (TEM) was done in the Central Microscopy Imaging Center at Stony Brook Medicine. Briefly, after embedding the kidney tissues in epoxy resin, ultrathin sections were stained with uranyl acetate and lead citrate, then mounted on a copper grid, and photographed under a FEI BioTwin G2 Transmission Electron Microscope with AMT XR-60 CCD digital Camera (FEI). Podocyte effacement was quantified as previously described (S. K. Mallipattu et al., (2017) J Am Soc Nephrol 28, 166-184).

u. IKKβ Kinase Activity

To measure IKKβ Kinase activity with or without BT503, the IKKβ Kinase Enzyme Systems with the ADP-Glo™ Assay (Promega) was utilized. Briefly, IKKβ were incubated with different concentration of BT503 or other IKKβ inhibitor or vehicle at room temperature for 10 minutes, and then a complete kinase reaction buffer (included 1× buffer, ATP, DTT and IKKtide) was added and further incubated for 1 hour. After the kinase reaction, an equal volume of ADP-Go™ Reagent is added to both terminate the kinase reaction and deplete the remaining ATP. Subsequently, the Detection Reagent is added to simultaneously convert ADP to ATP and measure the concentrations of newly synthesized ATP using a luciferase/luciferin reaction. The light generated is measured using a 96-well plate reader SpectraMax M3 (Molecular Devices). Luminescence can be correlated to ADP concentrations by using an ATP-to-ADP conversion curve.

v. Thermal Shift Assay

The cells were treated with BT503 (1 μM) for 1 hour, resuspended in PBS, and a 100 μl (˜200 k cells) was aliquoted. Each tube was treated at a range of temperatures (40° C. to 52° C.) for 3 minutes using Eppendorf Mastercycler gradient temperature setting. Protein was subsequently extracted from cells using freeze-thaw cycle, and IKKβ and β-actin were detected using western blot.

w. Toxicity Screen for BT503

BT503 was screened in hERG Human Potassium Ion Channel Cell Based QPatch CiPA Assay using CHO-K1 cells in collaboration with Eurofins Discovery Inc. After whole cell configuration is achieved, the cell is held at −80 mV. A 500 ms pulse to −40 mV is delivered to measure the leaking current, which is subtracted from the tail current on-line. The cell is subsequently depolarized to +40 mV for 500 ms and then to −80 mV over a 100 ms ramp to elicit the hERG tail current. The parameters measured were the maximum tail current evoked on stepping to 40 mV and ramping back to −80 mV from the test pulse. All data were filtered for seal quality, seal drop, and current amplitude. The peak current amplitude was calculated before and after compound addition and the amount of block was assessed by dividing BT503 current amplitude by the control current amplitude. Control is the mean hERG current amplitude collected 15 seconds at the end of the control; BT503 is the mean hERG current amplitude collected in the presence of BT503 at 3 and 10 μM.

Differentiated human podocytes were treated with BT503 (3, 10 μM), with comparison to DMSO and DEX (3, 10 μM) for 24 hours and oxygen consumption rate (OCR) and extracellular acidification rate (ECAR) were measured using a Seahorse XFe96 Analyzer (Agilent). Subsequently, basal OCR, maximal respiration, ATP-linked respiration, and spare respiratory capacity were determined in the presence of oligomycin, carbonyl cyanide-p-trifluoromethoxyphenylhydrazone (FCCP), and rotenone/antimycin A. All OCR measurements were normalized for the number of cells per well using CyQuant reagent (Life Technologies).

To test the toxicity profile in vivo, mice were initially treated with BT503 (IP, 0.5, 10 mg/kg) or DMSO (IP) for a 24-hour period. Serum was subsequently collected and mice were euthanized for collection of kidney, liver, and heart tissue.

x. Statistical Analysis

Student's t test was used to compare continuous data between two groups and two-way ANOVA with Tukey's post-test to compare continuous data between more than two groups. It could not assume normality on some data sets with smaller sample sizes, nonparametric statistical tests were performed using the Mann-Whitney test to compare continuous data between two groups and Kruskal-Wallis test with Dunn's post-test to compare continuous data between more than two groups. The exact test used for each experiment is denoted in the figure legends and data were expressed as the mean±SEM. All experiments were repeated a minimum of three times, and representative experiments are shown. Statistical significance was considered when p<0.05. All statistical analysis was performed using GraphPad Prism 9.0.

y. Study Approval

Stony Brook University Animal Institute Committee approved all animal studies and the NIH Guide for the Care and Use of Laboratory Animals was followed strictly.

z. Protein Target Selection and Modeling

Coordinates for docking ligands to IKKβ were obtained from the Protein Data Bank (pdb code 4KIK) (S. Liu et al., (2013) J Biol Chem 288, 22758-22767). The structure is an asymmetric dimer and both ATP binding sites contained the Staurosporine analog labeled K252a. Chain B of the dimer was a more complete structure and thus retained for modeling (S. Liu et al., (2013) J Biol Chem 288, 22758-22767). The program Chimera (E. F. Pettersen et al., (2004) J Comput Chem 25, 1605-1612), with the Modeller interface (A. Sali, T. L. Blundell, (1993) J Mol Biol 234, 779-815), was employed to fill in missing residues (180-183) on the activation loop (4KIK numbering) with no movement of flanking residues allowed. To simplify docking setup of the kinase domain, residues numbered greater than 316 and crystallographic waters were deleted. These deletions removed the majority of the central ubiquitin like domain and the entirety of the C terminal dimerization domain. A homologous protein-ligand structure was downloaded from the pdb (code 1GIH) (M. Ikuta et al., (2001) J Biol Chem 276, 27548-27554), comprised of a CDK2 construct, in which the ATP binding site was modified to mimic CDK4 and bound to the inhibitor named 1PU (1-[(9bR)-5-oxo-1,2,3,9b-tetrahydrobenzo[f]pyrrolizin-9-yl]-3-pyridin-2-yl-urea). The binding site in the CDK4 mimic from 1GIH shares high structural homology and reasonable sequence homology with IKKβ from 4KIK and the inhibitor 1PU share similarity with the inhibitors being investigated here, thus, the 1GIH structure provides a means to generate a second inhibitor-bound IKKβ complex (see results). To generate this second complex, as reference for docking, 1GIH was matched to 4KIK (backbone atoms) using the Chimera command ““matchmaker.”” As expected, the procedure yielded a well overlapped structure with low RMSD (RMSD=0.872 Å over 140 pruned atom pairs). The sequence homology for the group of residues closest to the binding site, defined here as ca. 8 Å from 1PU (N=53) was 37% by identity and 65% by similarity (Madeira et al. (2022) Nucleic Acids Res 50, W276-W279). The homology for the entire catalytic domains, defined here as residues 12 to 311 (4KIK numbering), was 25% identity and 39% similarity (calculations from EMBOSS Needle, www.ebi.ac.uk/Tools/psa/emboss_needle) (F. Madeira et al., (2022) Nucleic Acids Res 50, W276-W279).

aa. Protein Target and Ligand Setups Details

Protocols for preparing ligands and proteins for docking have previously been described (W. J. Allen et al., (2015) J Comput Chem 36, 1132-1156, S. Mukherjee, et al., (2010) J Chem Inf Model 50, 1986-2000). Briefly, ligand K252a (code 4KIK) was separated from IKKβ, protonated, and assigned partial atomic charges (AM1BCC method) (A. Jakalian, et al., (2002) J Comput Chem 23, 1623-1641) using the program Chimera (E. F. Pettersen et al., (2004) J Comput Chem 25, 1605-1612). The utility programs tleap and antechamber (J. Wang, et al., (2006) J Mol Graph Model 25, 247-260), from the AMBER (H. M. A. D. A. Case, et al., Amber 2023 University of California, San Francisco) suite of programs, were employed to re-assemble the complex and assign force field parameters (FF14SB (J. A. Maier et al., (2015) J Chem Theory Comput 11, 3696-3713) for protein, GAFF (J. Wang, et al., (2004) J Comput Chem 25, 1157-1174) for ligand). To relax the structure prior to docking, the K252a/IKKβ complex was subsequently minimized for 1000 steps using the AMBER module sander as part of the standard DOCK6 FLX (S. Mukherjee, et al., (2010) J Chem Inf Model 50, 1986-2000) preparation protocol. Other ligands employed for docking in this work, including 1PU (code 1GIH) (M. Ikuta et al., (2001) J Biol Chem 276, 27548-27554), INH14 (M. Drexel, J. Kirchmair, S. Santos-Sierra, (2019) Chembiochem 20, 710-717), and BT503, were prepared in a similar manner using Chimera and saved as MOL2 files.

bb. Dock Setups for the Binding Site

The energy-minimized protein was separated from the K252a/IKKβ complex, saved as a separate MOL2 file, and used to prepare files needed by DOCK6 (Allen et al. (2015) J Comput Chem 36, 1132-1156) for docking. Briefly, a protein molecular surface (unprotonated protein) was computed using the Chimera tool DMS, and then the DOCK utility sphgen (R. L. DesJarlais et al., (1988) J Med Chem 31, 722-729) was used to generate docking spheres on the molecular surface (I. D. Kuntz, et al., (1982) J Mol Biol 161, 269-288). Spheres within ca. 8 Å of the cognate ligand were saved to facilitate ligand anchor orientation in the binding site (I. D. Kuntz, et al., (1982) J Mol Biol 161, 269-288). Docking grids (grid program) (Shoichet et al. (1992) Journal of Computational Chemistry 13, 380-397), which help speed up the calculations, employed a 6-9 Lennard Jones potential for the van der Waals (VDW) term and a 4r distance dependent dielectric Coulombic potential for the electrostatic (ES) term. Grid dimensions were based on a bounding box of 8 Å from any docking sphere and employed a spacing resolution of 0.3 Å. Following these preparation steps, a DOCK energy minimization was performed for the theoretical complex of 1PU with IKKβ (from the 1GIH to 4KIK alignment) which served as a reference for the study. To facilitate molecular footprint calculations (discussed below) the DOCK multigrid (T. E. Balius et al. (2013) J Comput Chem 34, 1226-1240) protocol was used to isolate key residues involved in protein ligand binding.

cc. Docking Protocol—Grid Score Only

To complement the energy minimizations, pose reproduction calculations were also performed (FLX protocol) for K252a with 4KIK and 1PU with 1GIH. 1PU was docked into IKKβ using the minimized aligned structure as the ““theoretical reference.”” Pose reproduction experiments employed equal weighting of VDW and ES terms, with each experiment generating 100 conformers for each ligand with the top one being retained for geometric comparisons and molecular dynamics (MD) simulations.

dd. Docking Protocol—Grid Score with Restraints

For INH14 and BT503 docking, a similarity-based function comprised of Hungarian Matching Similarity (HMS) (W. J. Allen, R. C. Rizzo, (2014) J Chem Inf Model 54, 518-529) and Volume Overlap Similarity (VOS) (G. M. Sastry, et al., (2011) J Chem Inf Model 51, 2455-2466) terms was employed (along with grid score) to help bias sampling in IKKβ towards the conformation adopted by the aligned 1PU reference which makes key H-bond interactions with the backbone of Cys099 (see Results herein). The weights employed for VDW, ES, HMS, and VOS terms were 1, 1, 5, and −5, respectively. The above scoring function was used during orienting, minimization, dihedral sampling, as well as final rank ordering. The VDW and ES terms were assessed to make sure the above weightings did not produce unlikely conformations for the top ranked pose. As before, 100 conformers were saved for each ligand, with the top pose being retained for molecular dynamics simulations. Molecular footprints (Balius et al. (2013) J Comput Chem 34, 1226-1240; Balius et al. (2011) J Comput Chem 32, 2273-2289) for the docked and reference ligands, based on the most favorable per-residue interaction energy decomposition (VDW and ES terms) observed across the “collective” group of poses in IKKβ were also computed.

ee. Minimization, Equilibration, and Production (Molecular Dynamics)

Docked and reference poses used for MD simulations were prepared as above using the AMBER utility programs tleap and antechamber (J. Wang et al. (2006) J Mol Graph Model 25, 247-260 (2006)) to assemble coordinates and assign the required force fields (FF14SB (J. A. Maier et al. (2015) J Chem Theory Comput 11, 3696-3713) for protein, GAFF (Wang et al. (2004) J Comput Chem 25, 1157-1174) for ligand). Each complex was solvated with an octahedron of 12 Å TIP3P (W. L. Jorgensen et al. (1983) J Chem Phys 79, 926-935) waters and neutralized as appropriate with sodium and chloride counter ions. A previously described nine-step protocol (Y. C. Zhou et al. (2019) Biochemistry-Us 58, 4304-4316) was then employed to minimize and equilibrate each complex prior to running production MD.

Briefly, a restrained energy minimization followed by short MD (100 ps) was performed (steps 1-2, 5 kcal/mol-Å-2 restraint, heavy atoms only) followed by three minimizations in which the weights were decreased from 2 to 0.1 to 0.05 kcal/mol-Å-2 (steps 3-5, heavy atoms only). Three additional short MD equilibrations (100 ps each) were performed in which the weights were decreased from 1 to 0.5 to 0.1 kcal/mol-Å-2 (step 6-8, heavy atoms only). A final step of MD equilibration was performed with a restraint of 0.1 kcal/mol-Å-2 applied only to the protein backbone (step 9). Production MD employed a 1 kcal/mol-Å-2 backbone only restraint. For each system, GPU accelerated MD (R. Salomon-Ferrer et al. (2013) Journal of Chemical Theory and Computation 9, 3878-3888) was performed for 10 nanoseconds. Chimera was used to visually assess simulation outcomes and the 3D coordinate analysis program cpptraj (D. R. Roe, T. E. Cheatham, (2013) Journal of Chemical Theory and Computation 9, 3084-3095) was used to calculate ligand RMSDs relative to originally docked poses. Cpptraj was also used to extract evenly spaced snapshots (frames) from the production MD trajectories, and the ambpdb and antechamber (J. Wang, et al., (2006) J Mol Graph Model 25, 247-260 (2006), H. M. A. D. A. Case, et al., Amber 2023 University of California, San Francisco) utilities were used to convert snapshots to MOL2 format (Sybyl atom types), so that time averaged footprints could be estimated using DOCK6 (W. J. Allen et al., (2015) J Comput Chem 36, 1132-1156). Energy minimizations of each frame (ligand restraint weight=10 kcal/mol-Å-2) were performed to account for any potential differences in force field parameters in switching from DOCK to AMBER format. Time averaged footprints computed using DOCK6 employed the same residue list as determined from the original docking calculations but were performed in Cartesian space (6-12 LJ potential, 4r distance dependent dielectric).

### 2. Identification and Validation of Small Molecule Inducers of KLF15 in Cultured Human Podocytes

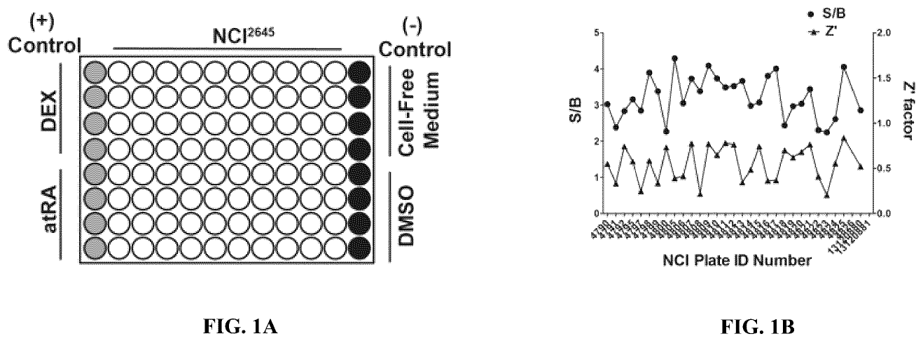

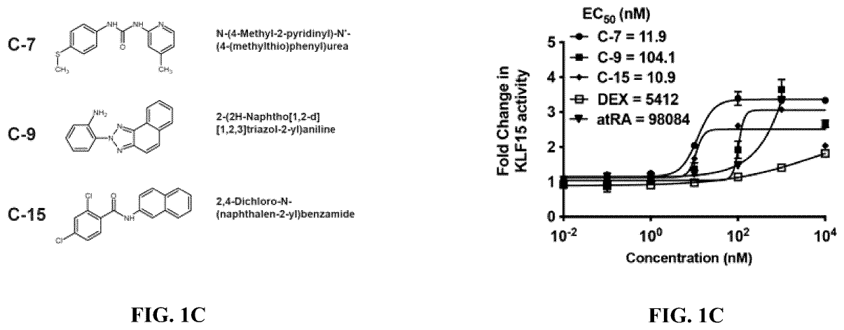

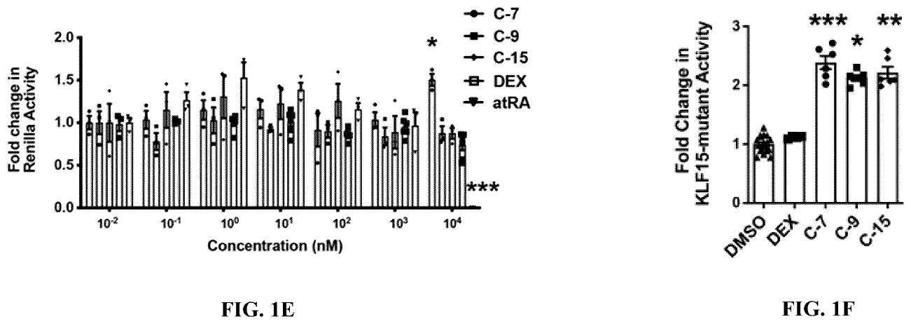

To identify small molecule inducers of KLF15 expression in human podocytes, human podocytes were generated with a dual reporter, firefly luciferase directed at the KLF15 promoter region and renilla luciferase (pEZX-FR03hKLF15p). Subsequently, pEZX-FR03hKLF15p human podocytes was utilized to develop a cell-based high-throughput screening (HTS) assay in a 96-well layout under nonpermissive conditions (FIG. 1A) to screen the National Cancer Institute Drug Screening Sets (2,645 compounds) at a final concentration of 1 μM in 1% DMSO for 24 hours. The positive controls were inducers of KLF15 expression, dexamethasone (DEX) and all-trans retinoic acid (atRA), and media-free and cell-free wells served as negative controls. The HTS podocyte assay exhibited high reproducibility with low variability as determined by a high signal to background (S/B) 3.22 and a low Z-score (Z′)˜0.56 (FIG. 1B). Using the traditional hit threshold selection methodology (A. B. Bialkowska, et al., (2009) Mol Cancer Ther 8, 563-570), 44 hits were identified with greater than 2.5-fold change in KLF15 reporter activity (Table 2). These initial hits from this primary screen belong to several classes of small molecules including benzamides, urea analogues, aromatic amines, nitriles, thiols and dienone. Dose-response studies (concentration range from 0.1 nM to 10 μM) were subsequently conducted for all 44 hit compounds and identified 16 small molecules with EC₅₀<100 nM (Table 2). Based on the composition of low-nanomolar EC₅₀, stable cell viability, and the Lipinski's rule of five (evaluate druggability and the likelihood of the compound being orally active), C-7, C-9, C-15 were advanced as KLF15 agonists (FIG. 1C-1E, Table 2, and Table 3). In comparison, DEX had an EC₅₀ of 5412 nM, with a trend towards lower renilla activity at higher doses, suggesting cell toxicity as compared to the KLF15 agonists C-7, C-9, and C-15 (FIG. 1D and FIG. 1E). Conversely, C-7 showed a significant increase in renilla activity with stable KLF15 activity at higher concentrations (10⁴ nM) as compared to all other groups. Since KLF15 is mediator of glucocorticoid (GC) signaling and glucocorticoid response elements (GRE) occupy the promoter region of KLF15 (Mallipattu and He (2016) Am J Physiol Renal Physiol 311, F46-51; Asada et al. (2011) Lab Invest 91, 203-215; Sasse et al. (2013) Mol Cell Biol 33, 2104-2115 (2013)), a podocyte dual reporter was generated with a mutation in the GRE (pEZX-FR03hKLF5p-mutant) to test whether the induction of KLF15 activity is independent of GR signaling. All three KLF15 agonists induced similar KLF15 activity in pEZX-FR03hKLF5p-mutant podocytes as compared to DMSO, which was lost with DEX treatment (FIG. 1F). Collectively, this developed human podocyte KLF15 HTS reporter assay was used to identify and validate small molecule inducers of KLF15, independent of GC signaling.

TABLE 2

| | | | | Fold | EC₅₀ |
|---|---|---|---|---|---|
| Hits | NSC | Pubchem | Description | Change | (nM) |
| C-1 | 33004 | 5351155 | Phenol,4-(2-benzothiazolyl) | 3.0083 | 99.63 |
| C-2 | 320846 | 71750 | Batracyclin | 3.0717 | 14.89 |
| C-3 | 80087 | 5784745 | [(4-Dimethylamino)benzylidene]indene | 2.6455 | 17.73 |
| C-4 | 667251 | 5351425 | 2-Propenenitrile, 3-[3- | 2.5670 | 23.74 |
| | | | (dimethylamino)phenyl]-2-phenyl- | | |
| C-5 | 159031 | 292850 | 5-methoxy-2-phenyl-1H-indole | 3.1354 | 55.88 |
| C-6 | 20619 | 3003755 | 1,3-bis(3-methylpyridin-2-yl)thiourea | 2.7113 | 770.50 |
| C-7 | 142269 | 285402 | 1-(4-Methylpyridin-2-yl)-3-(4- | 3.1695 | 11.90 |
| | | | methylsulfanylphenyl)urea | | |
| C-8 | 214029 | 310541 | 3-fluoro-N-(2-naphthyl)benzamide | 2.7914 | 70.53 |
| C-9 | 158549 | 292642 | 2-benzo[e]benzotriazol-2-ylaniline | 2.7182 | 76.26 |
| C-10 | 305743 | 65758 | Pifexole | 2.7219 | 332.40 |
| C-11 | 343557 | 335175 | 2-amino-N-(2,4- | 2.6436 | 705.90 |
| | | | dichlorophenyl)benzamide | | |
| C-12 | 33738 | 234249 | 2-[2-(3,4-Dichloroanilino)-2- | 3.1347 | 37.28 |
| | | | oxoethoxy]benzamide | | |
| C-13 | 522131 | 351549 | N-(3,4-dichlorophenyl)-N′-(2- | 2.6384 | 86.12 |
| | | | hydroxyphenyl)urea | | |
| C-14 | 164435 | 295274 | 1-(4-Anilinophenyl)-3-(2- | 3.0342 | 42.10 |
| | | | chlorophenyl)urea | | |
| C-15 | 158959 | 292795 | 2,4-dichloro-N-(naphthalen-2- | 2.9715 | 10.93 |
| | | | yl)benzamide | | |
| C-16 | 177407 | 5383615 | 5,6-dichloro-2-[3- | 2.8171 | 52.14 |
| | | | (trifluoromethyl)phenyl]-1H- | | |
| | | | imidazo[4,5-b]pyrazine | | |
| C-17 | 353527 | 8137375 | Tin(IV), chlorotriphenyl[1-(4- | 4.6159 | NA |
| | | | ethoxyphenyl)-3-cyanoureato]-, | | |
| | | | hydrogen, trimethylamine | | |
| C-18 | 693632 | 5351439 | 2-(2-(1-(pyrimidin-4- | 3.2863 | 156.50 |
| | | | yl)ethylidene)hydrazinyl)benzo[d]thiazole | | |
| C-19 | 70931 | 122724 | Celastrol | 3.2798 | 11.18 |
| C-20 | 78130 | 105268 | 3-[(4-Methoxyphenyl)azo]-2,6- | 3.1426 | 84.44 |
| | | | pyridinediamine | | |
| C-21 | 82025 | 255922 | 2-hydroxy-N-(4-methyl-2-nitrophenyl)- | 3.1125 | 61.35 |
| | | | 3-nitrobenzamide | | |
| C-22 | 757441 | 6450551 | Axitinib | 3.0642 | ~28669 |
| | | | | | 3 |
| C-23 | 184403 | 5351258 | 5-Methylbenzo[c]phenanthridin-5-ium- | 3.0541 | 296.00 |
| | | | 2,3,8,9- | | |
| | | | tetrol; pyridine; chloride; hydrochloride | | |
| C-24 | 21683 | 228624 | (4Z)-5-imino-4-[(4- | 3.0292 | 2670.00 |
| | | | methylphenyl)hydrazinylidene]-1- | | |
| | | | phenylpyrazol-3-amine | | |
| C-25 | 680516 | 387118 | N,N,3-trimethyl-4-[(E)-6- | 3.0228 | 15.77 |
| | | | quinolylazo]aniline | | |
| C-26 | 658350 | 135462447 | 2-[2-[(2E)-2-[(5Z)-5- | 3.0168 | 484.20 |
| | | | (anilinomethylene)-3-ethyl-4-oxo- | | |
| | | | thiazolidin-2-ylidene[hydrazino]thiazol- | | |
| | | | 5-yl]-N-(o-tolyl)acetamide | | |
| C-27 | 640584 | 5351382 | 3-(3,4-dichlorophenyl)-1-(2-(3,5- | 2.8899 | NA |
| | | | diphenyl-1H-pyrazol-1-yl)-4- | | |
| | | | methylthiazol-5-yl)prop-2-en-1-one | | |
| C-28 | 680515 | 39164 | 7-(p-N,N- | 2.8704 | ~6310 |
| | | | Dimethylaminophenylazo)benzofuran | | |
| C-29 | 7071729 | 4204363 | s-Triazolo[4,8-methyl | 2.8700 | 549.80 |
| C-30 | 106461 | 267294 | 2,5-dipyridin-2-yl-1,3,4-thiadiazole | 2.8592 | 334.80 |
| C-31 | 634224 | 366050 | 1-Indenone, 3-hydroxy-2-(2- | 2.8466 | 45.86 |
| | | | quinoxalinyl)- | | |
| C-32 | 658293 | 5351418 | ethyl (2Z)-5-methyl-2-[(4- | 2.8360 | NA |
| | | | nitrophenyl)methylidene]-3-oxo- | | |
| | | | [1,3]thiazolo[2,3-b][1,3]thiazol-4-ium- | | |
| | | | 6-carboxylate;chloride | | |
| C-33 | 33005 | 95746 | (4E)-4-(3H-1,3-benzothiazol-2- | 2.7511 | 490.10 |
| | | | ylidene)-3-hydroxycyclohexa-2,5-dien- | | |
| | | | 1-one | | |
| C-34 | 635404 | 366687 | 2-hydroxy-N′-(2-oxo-1,2-dihydro-3H- | 2.6900 | 121.60 |
| | | | indol-3-ylidene)benzohydrazide | | |
| C-35 | 367416 | 339701 | 7-Methoxy-1,4,6- | 2.6597 | 319.20 |
| | | | benzotriazaphenothiazine | | |
| C-36 | 176367 | 708470 | N-(pyridin-2- | 2.6427 | 291.90 |
| | | | ylcarbamothioyl)benzamide | | |
| C-37 | 9358 | 81528 | 4-(2-pyridylazo)-n,n-dimethylaniline | 2.6148 | 26.96 |
| C-38 | 50648 | 242249 | 2-<3-Nitro-phenylcarbamoyl>-naphth- | 2.6069 | 144.80 |
| | | | 1-ol | | |
| C-39 | 289748 | 324415 | N,N-dimethyl-4-[(4-methyl-3-oxido- | 2.5988 | ~6890 |
| | | | 1,3-thiazol-3-ium-2-yl)diazenyl]aniline | | |
| C-40 | 130872 | 6164010 | 2-(4-methoxyphenyl)-3-pyridin-2- | 2.5975 | 545.50 |
| | | | ylprop-2-enenitrile | | |
| C-41 | 647136 | 372186 | 1-Methoxy-7,8-dimethylphenazine | 2.5935 | 214.90 |
| C-42 | 657598 | 5351414 | Thiazolo[2,3-b]thiazolium, 2,3-dihydro- | 2.5844 | NA |
| | | | 2-[(4-nitrophenyl)methylene]-5-(3- | | |
| | | | nitrophenyl)-3-oxo-, chloride | | |
| C-43 | 400938 | 135441716 | 4-[2-(4- | 2.5682 | 866.00 |
| | | | methylphenyl)hydrazinyl]benzotriazol- | | |
| | | | 5-one | | |
| C-44 | 164880 | 5937213 | 1-[2-(2,3- | 2.5631 | ~1839 |
| | | | Dimethoxyphenyl)vinyllisoquinoline | | |

TABLE 3

| KLF15 Agonist | C-7 | BT501 | BT502 | BT503 |
|---|---|---|---|---|
| Physiochemical Properties | | | | |
| MW (g/mol) | 273.35 | 259.32 | 284.34 | 273.35 |
| # heavy atoms | 19 | 18 | 20 | 19 |
| # aromatic heavy atoms | 12 | 12 | 12 | 12 |
| # rotational bonds | 5 | 4 | 5 | 5 |
| # H-bond acceptors | 2 | 2 | 3 | 2 |
| # H-dond donors | 2 | 2 | 2 | 2 |
| Lipophilicity (XLOGP3) (−0.7 | 2.62 | 2.78 | 1.98 | 2.33 |
| to +5.0) | | | | |
| Polarity (TSPA) (20 to 130 Å²) | 79.32 | 74.63 | 103.11 | 79.32 |
| Solubility (log S) (not higher | −3.32 | −3.43 | −2.96 | −3.14 |
| than 6) | | | | |
| Pharmacokinetics | | | | |
| GI absorption | High | High | High | High |
| BBB permeability | No | Yes | No | No |
| Druglikeness | | | | |
| Lipinski | Yes | Yes | Yes | Yes |
| Ghose | Yes | Yes | Yes | Yes |
| Veber | Yes | Yes | Yes | Yes |
| Egan | Yes | Yes | Yes | Yes |
| Muegge | Yes | Yes | Yes | Yes |
| Bioavailability score | 0.55 | 0.55 | 0.55 | 0.55 |
| Leadlikeness | Yes | Yes | Yes | Yes |

Referring to FIG. 1A-F, identification of hit KLF15 agonists from NCI²⁶⁴⁵ HTS is demonstrated. FIG. 1A depicts a 96-well plate format for NCI2645 HTS. First column, (+) control (DEX- and atRA-treated podocytes); Last column, (−) control (cell free medium only, DMSO-treated cells); All other columns, cells treated with NCI2645 compounds with a final concentration of 1 μM in 0.5% DMSO. FIG. 1B shows variations in S/B ratios and Z′ factors across plates in NCI2645 HTS. S/B ratios and Z′ factors were calculated for each plate and plotted in the graphs. Left, y axis, S/B ratios; right, Z′ factors. FIG. 1C shows representative chemical structures for C-7, C-9, C-15. FIG. 1D refers to dose-response of KLF15 reporter activity for C-7, C-9, C-15, DEX, and atRA at various concentrations (relative to DMSO, normalized to renilla). EC₅₀ was calculated for each compound. FIG. 1E shows representative fold change in renilla activity for C-7, C-9, C-15, DEX, and atRA at various concentrations (relative to DMSO) (n=4, *p<0.05, ***P<0.001, Kruskal-Wallis test with Dunn's post-test). FIG. 1F shows representative fold change in KLF15-mutant reporter activity (relative to DMSO, normalized to Renilla) (n=8, *p<0.05, **p<0.01, ***p<0.001, Kruskal-Wallis test with Dunn's post-test).

### 3. KLF15 Agonists Attenuate Podocyte Injury Under Cell Stress

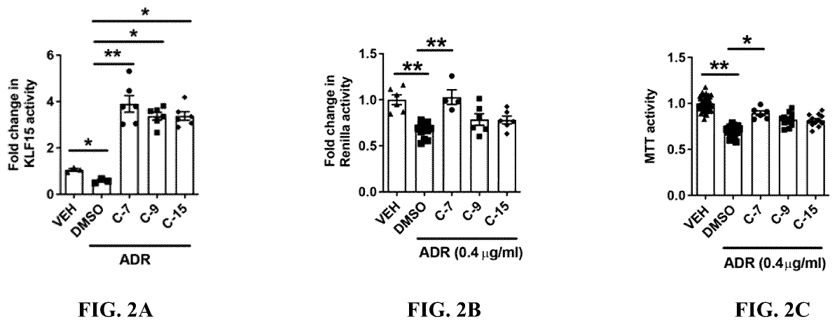

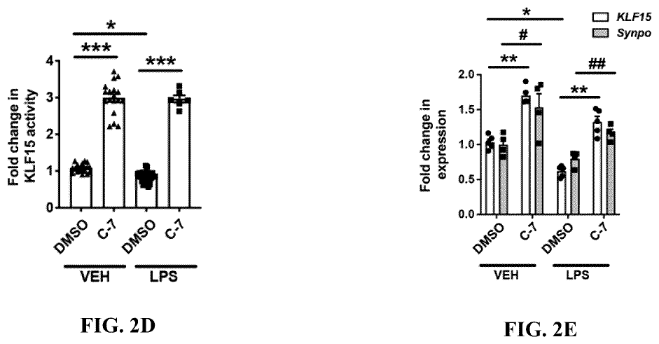

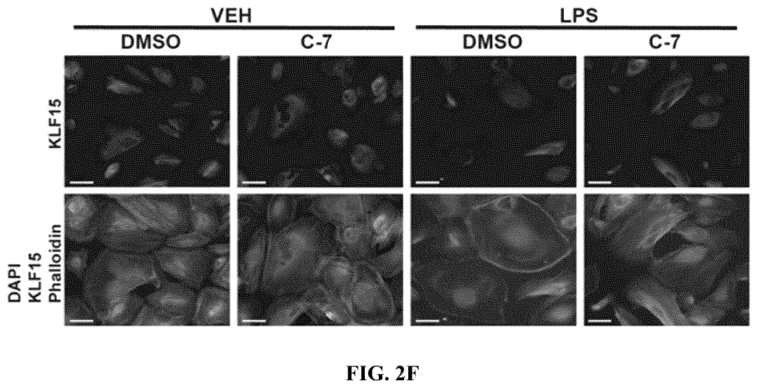

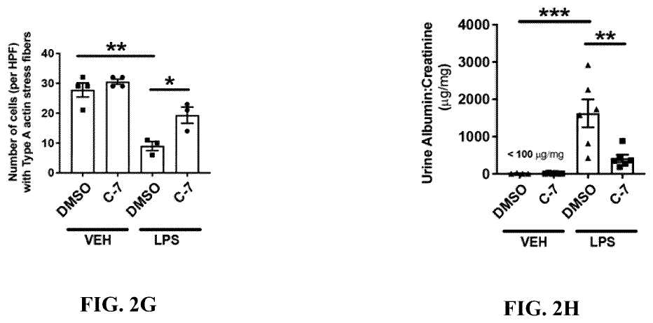

To determine the therapeutic efficacy of these KLF15 agonists (C-7, C-9, C-15), cultured human podocytes were initially treated with all three agonists as compared to DMSO in the setting of adriamycin (ADR). All three agonists induced KLF15 activity as compared to DMSO-treated podocytes, which showed a reduction in KLF15 activity (FIG. 2A). In addition, cell viability (measured by renilla activity and MTT assay) was only maintained with C-7 as compared to DMSO-treated podocytes (FIG. 2B and FIG. 2C). Based on these data, C-7 was advanced to begin in silico pharmacokinetic (PK) profiling and for further testing in additional in vitro and in vivo models of podocyte injury. Utilizing the open-source Swiss Institute of Bioinformatics to predict the pharmacokinetics, “leadlikeness,” and medicinal chemistry, C-7 demonstrated water solubility (Log S˜3.32), moderate lipophilicity (Log P ˜2.64), high GI absorption, impermeability to blood-brain barrier, and high druggability (based on Lipinski, Ghose, Veber, Egan tests) (Daina et al. (2017) Sci Rep 7, 42717), without wishing to be bound by theory suggesting a high prediction to be a “target-to-hit” compound (Table 3). Using a second model of podocyte injury, C-7 similarly maintained KLF15 activity as compared to DMSO in the setting of lipopolysaccharide (LPS) treatment (FIG. 2D). In addition, C-7 restored KLF15 and Synaptopodin expression as compared to DMSO-treated podocytes under LPS conditions (FIG. 2E). While LPS reduced KLF15 expression and destabilized the actin cytoskeleton (measured by actin stress fiber formation), immunostaining showed that C-7 restored KLF15 expression and actin stress fiber formation (FIG. 2F, 2G). Furthermore, using the LPS proteinuric murine model (Mallipattu et al. (2017) J Am Soc Nephrol 28, 166-184; Mallipattu et al. (2012) J Biol Chem 287, 19122-19135; Reiser et al. (2004) J Clin Invest 113, 1390-1397; Lee et al. (2015) J Am Soc Nephrol 26, 2741-2752), concurrent treatment with C-7 significantly abrogated albuminuria as compared to DMSO (FIG. 2H). Without wishing to be bound by theory, these data suggest that the “target-to-hit” small molecule, C-7, attenuated podocyte injury and albuminuria in the setting of cell stress.

Referring to FIG. 2A-H, KLF15 agonists attenuate podocyte injury in the setting of podocyte stress. FIG. 2A shows representative fold change in KLF15 reporter (relative to VEH-treated podocytes, normalized to renilla) (n=6, *p<0.5, **p<0.01, Kruskal-Wallis test with Dunn's post-test), FIG. 2B shows representative Renilla activity (relative to VEH-treated podocytes) (n=6, **p<0.01, Kruskal-Wallis test with Dunn's post-test), and FIG. 2C shows representative MTT activity in DMSO-, C-7-, C-9-, C-15-treated podocytes in the setting of ADR treatment (n=12, *p<0.5, **p<0.01, Kruskal-Wallis test with Dunn's post-test). FIG. 2D shows representative fold change in KLF15 reporter activity in DMSO- and C-7-treated podocytes in the setting of LPS treatment (n=6-8, *p<0.5, ***p<0.001, Kruskal-Wallis test with Dunn's post-test). FIG. 2E shows representative fold change in KLF15 and synaptopodin (synpo) expression in DMSO- and C-7-treated podocytes in the setting of LPS treatment (n=5, *p<0.5, **p<0.01 (KLF15), #p<0.5, ##p<0.01 (Synpo), Kruskal-Wallis test with Dunn's post-test). FIG. 2E shows representative immunostaining for phalloidin, KLF15, and Hoechst. Representative images from 3 independent experiments are shown. FIG. 2G shows quantification of Type A actin stress fibers in DMSO- and C-7-treated podocytes in the setting of LPS treatment (n=3-4, *p<0.5, **p<0.01, Kruskal-Wallis test with Dunn's post-test). FIG. 2A shows representative urine albumin:creatinine ratio in DMSO- and C-7-treated mice in the setting of LPS administration (n=6, **p<0.01, ***p<0.001, Kruskal-Wallis test with Dunn's post-test).

### 4. Novel Structural Analogues of C-7 Restores KLF15 Reporter Activity and Podocyte Viability Under Cell Stress

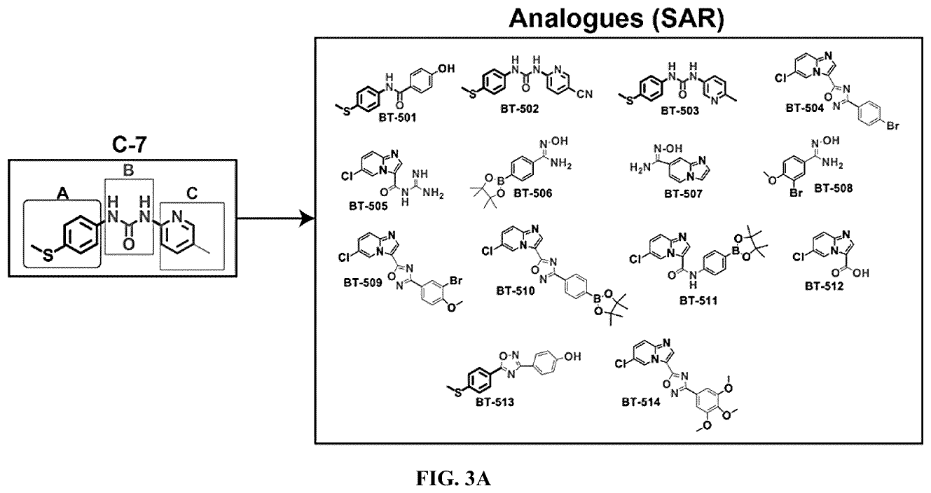

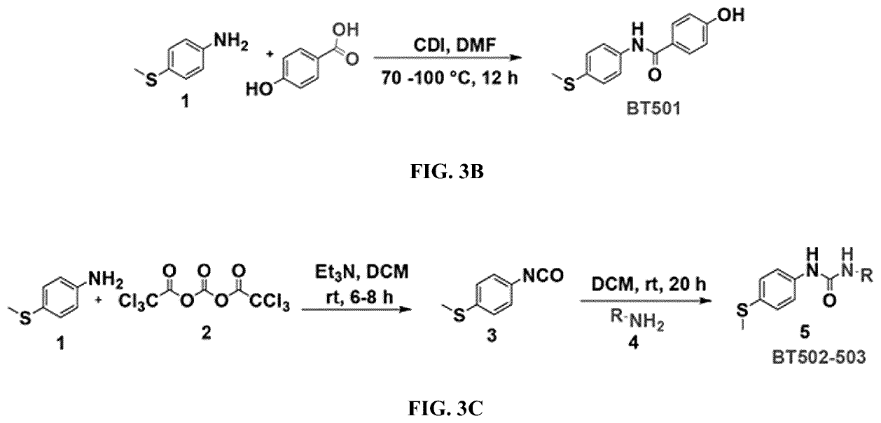

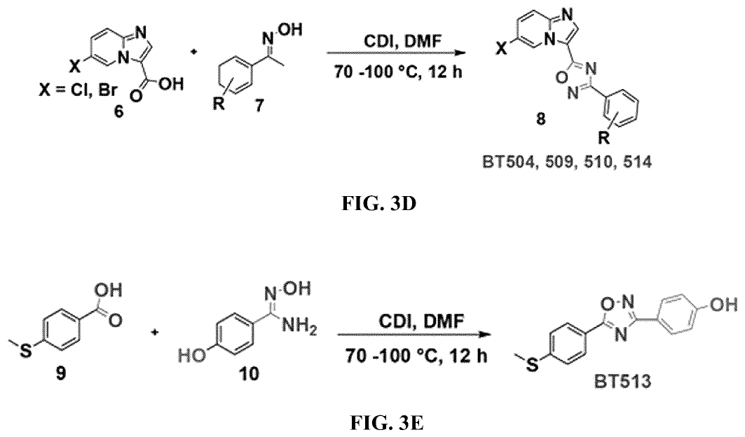

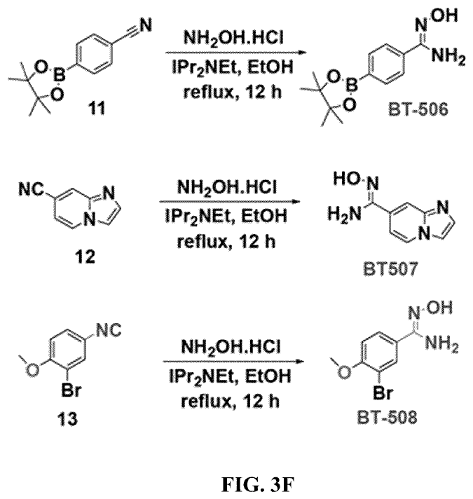

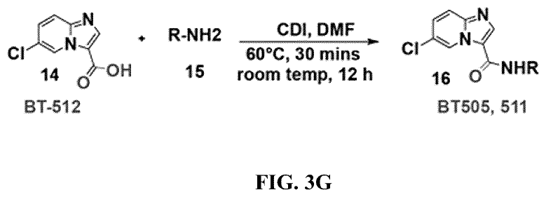

Based on the preceding low EC₅₀, “leadlikeness,” and salutary effects in podocytes in vitro and in vivo, C-7 was selected for a Structure Activity Relationship (SAR) study to synthesize novel structural analogues with a goal of improving therapeutic efficacy without compromising cell toxicity. 14 analogues were generated by targeting the 3 structural moieties of C-7 (Part A contains 4 substituted thiomethyl group of phenyl ring system, Part B contains substituted urea derivative and Part C contains substituted pyridine derivatives) as described herein (FIG. 3A-G).

Referring to FIG. 3A-G, a Structure Activity Relationship (SAR) Study of C-7 was conducted by modifying different core moieties. Referring to FIG. 3A, SAR was conducted on C-7 with 3 structural moieties (Part A contains 4 substituted thiomethyl group of phenyl ring system, Part B contains substituted urea derivative and Part C contains substituted pyridine derivatives) to generate 15 novel analogues. FIG. 3B shows a representative synthetic scheme for amide analogue, BT501. FIG. 3C shows a representative general synthetic scheme for C-7 analogues (BT502 and BT503) for substituting the pyridine ring. FIG. 3D shows a general procedure for a synthesis to replace the urea derivatives (in ring Part B) with oxadiazole (BT504, BT509, BT510, BT514). FIG. 3E shows a representative synthetic scheme for BT513. FIG. 3F shows a representative synthetic scheme for BT506, BT507 and BT508. A general procedure for the synthesis of amide analogues (BT505, BT511) is shown in FIG. 3G.

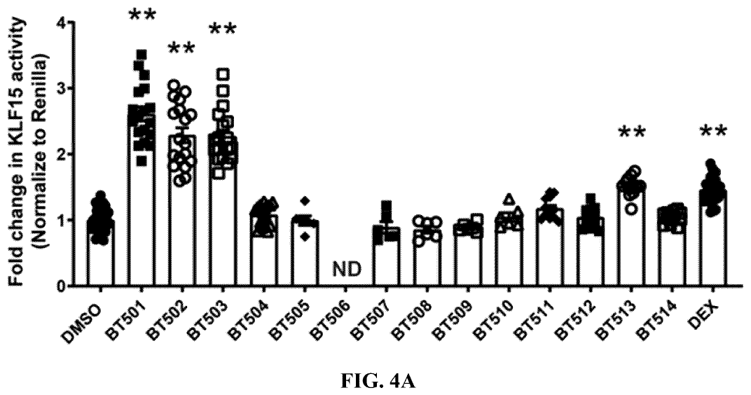

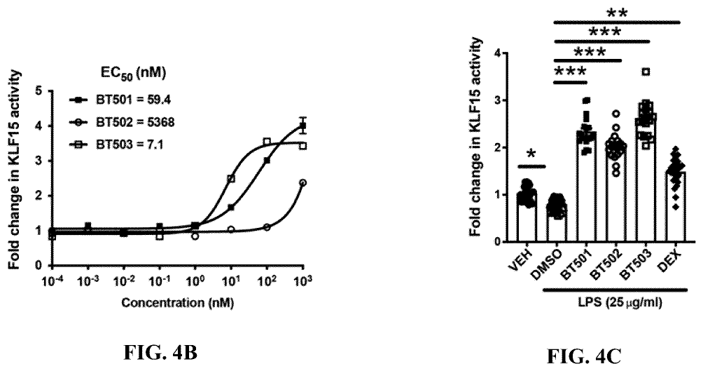

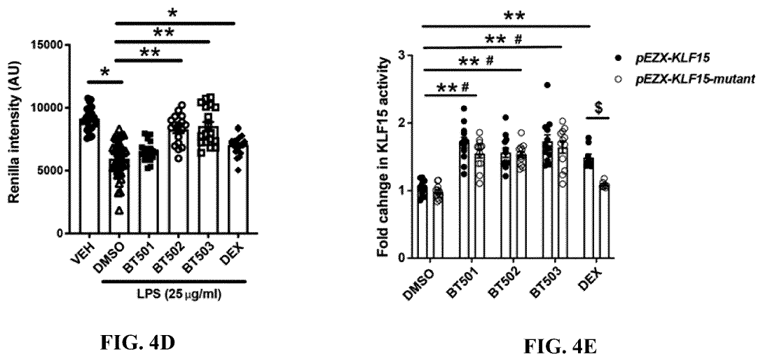

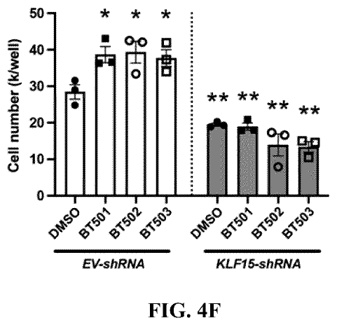

These novel analogues were screened and determined that BT501, BT502 and BT503 significantly induced KLF15 reporter activity (>2-fold), with no observed toxicity to human podocytes) as compared to DMSO-treated podocytes (FIG. 4A). In addition, all three analogues had higher KLF15 activity as compared to DEX-treated podocytes. Subsequent dose escalation studies for these 3 analogues determined BT503 with EC₅₀˜7.1 nM, lowest as compared to the other analogues and most closely resembling C-7 (FIG. 4B). While LPS reduced reporter activity in DMSO-treated podocytes, BT501, BT502, and BT503 maintained the elevated KLF15 activity in the setting of LPS treatment (FIG. 4C). However, only BT502 and BT503 restored renilla activity (i.e., cell viability) as compared to DMSO-treated podocytes in the setting LPS treatment (FIG. 4D). Similar to C-7, all three novel analogues induced KLF15 activity as compared in DMSO and DEX in pEZX-KLF15-GRE podocytes, suggesting these novel compounds induced KLF15 activity independent of GC signaling (FIG. 4E). To test the specificity of these novel analogues to KLF15, podocytes were generated with KLF15 knockdown (KLF15-shRNA) and compared them to control podocytes (EV-shRNA). While all three analogues improved cell survival in EV-shRNA podocytes, this was lost in KLF15-shRNA podocytes (FIG. 4F). Without wishing to be bound by theory, these data suggest that SAR study of the “target-to-hit” small molecule, C-7, identified novel structural analogues (BT501, BT502, and BT503) that induced KLF15 reporter activity and restored podocyte survival, independent of GC signaling.

Referring to FIG. 4A-F, Novel lead compounds restore KLF15 activity in the setting of podocyte stress. FIG. 4A shows representative fold change in KLF15 reporter activity (relative to DMSO, normalized to Renilla, ND—not determined due to low solubility) (n=6-12, **p<0.01, Kruskal-Wallis test with Dunn's post-test). FIG. 4B shows representative dose-response of KLF15 reporter activity for BT501, BT502, and BT503 at various concentrations (normalized to renilla). EC₅₀ was calculated for each compound. FIG. 4C shows representative fold change in KLF15 reporter activity (relative to DMSO, normalized to Renilla) in the setting of LPS treatment (n=18, *p<0.05, **p<0.01, ***p<0.001, Kruskal-Wallis test with Dunn's post-test). FIG. 4D shows Renilla intensity in the setting of LPS treatment (n=18, *p<0.05, **p<0.01, Kruskal-Wallis test with Dunn's post-test). FIG. 4E shows representative fold change in pEZX-KLF15 and pEZX-KLF15-mutant reporter activity (relative to DMSO, normalized to Renilla) (n=12, **p<0.01 (pEZX-KLF15 activity), ^{#}p<0.05 (pEZX-KLF15-mutant activity), Kruskal-Wallis test with Dunn's post-test; ^{$}p<0.05, Mann-Whitney Test). FIG. 4F shows representative data for cell number for EV-shRNA and KLF15-shRNA podocytes (n=3, *p<0.05, **p<0.01, Kruskal-Wallis test with Dunn's post-test).

### 5. BT503 Attenuates Albuminuria and Kidney Disease in Mice

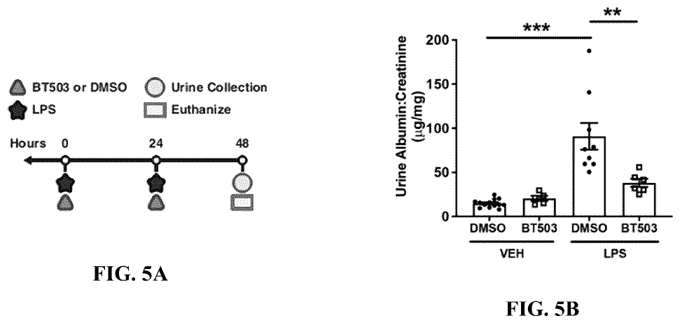

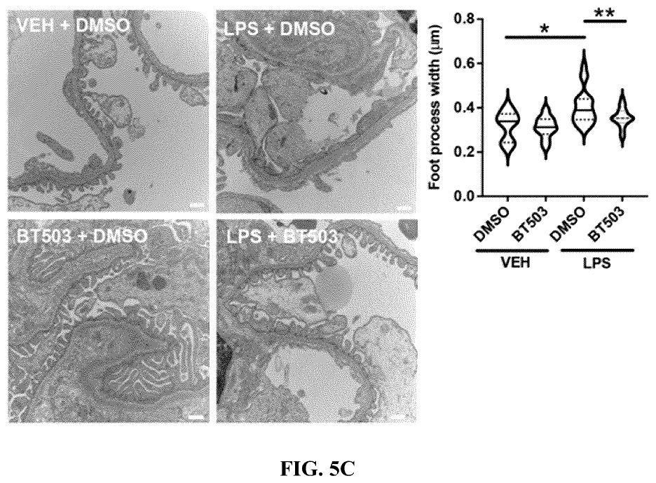

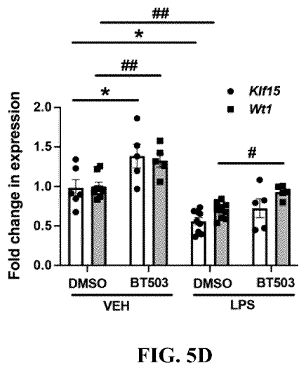

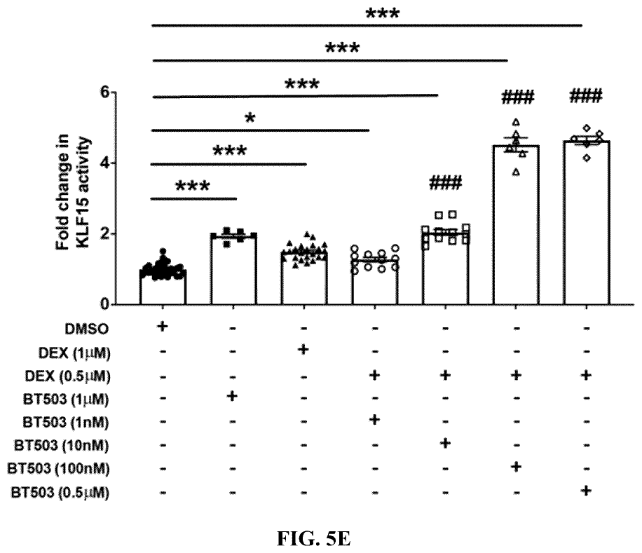

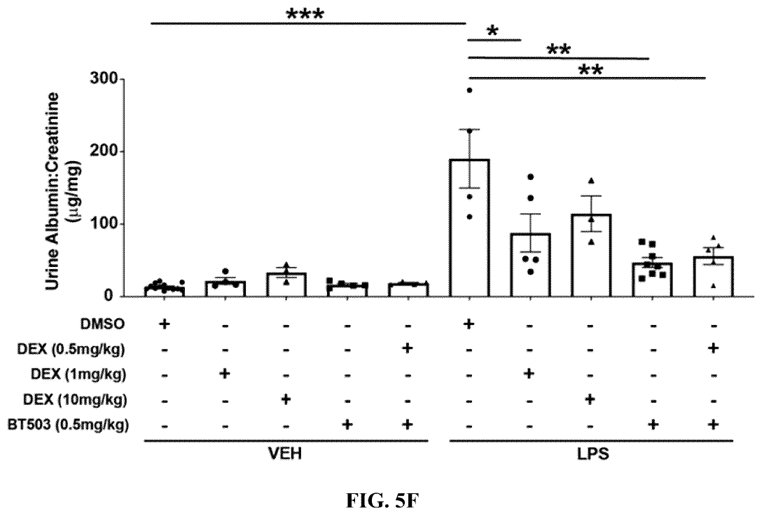

Since BT503 exhibited an EC₅₀<10 nM and improved cell viability in podocytes under cell stress, BT503 was utilized to test its salutary effects in vivo using proteinuric murine models. Initially, utilizing the short-term LPS proteinuric murine model (FIG. 5A), concurrent treatment with BT503 attenuated albuminuria as compared to DMSO-treated mice (FIG. 5B). In addition, LPS treatment increased foot process effacement (i.e., podocyte injury), which was significantly restored in BT503-treated mice (FIG. 5C). Glomerular Klf15 and Wilms Tumor 1 (Wt1) expression was also restored in BT503-treated mice as compared to DMSO-treated mice under LPS conditions (FIG. 5D).

GCs remains the initial treatment for primary glomerulopathies such as MCD and primary FSGS (M. van Husen, M. J. Kemper, (2011) Pediatr Nephrol 26, 881-892). Since BT503 induced KLF15 activity independent of GC signaling, determining whether the salutary effects of BT503 could be synergistic to GCs and reduce the GC dose necessary to maintain therapeutic efficacy was explored. DEX has been previously reported to induce KLF15 expression directly in podocytes (Mallipattu and He (2016) Am J Physiol Renal Physiol 311, F46-51; Asada et al. (2011) Lab Invest 91, 203-215; Sasse et al. (2013) Mol Cell Biol 33, 2104-2115). While an increase in KLF15 activity with DEX (1 μM) was observed, the combination of BT503 (1 nM, 10 nM) and a reduced dose of DEX, 50%, maintained similar therapeutic efficacy as either DEX alone (1 μM) or BT503 (1 μM) (FIG. 5E). Interestingly, maintaining this reduced dose of DEX (0.5 μM) in combination with BT503 at higher doses (100 nM, 0.5 μM) had a synergistic effect on KLF15 activity as compared to either alone at double the concentration (1 μM) (FIG. 5E). Furthermore, the combination of BT503 and reduced DEX (0.5 μg/g) maintained a similar reduction in albuminuria as compared to either BT503 (0.5 μg/g) or DEX (1 or 10 μg/g) post-LPS treatment (FIG. 5F). Without wish to be bound by theory, these data suggest that BT503 ameliorates albuminuria and podocyte injury and reduces the dose of DEX required to maintain therapeutic efficacy.

Referring to FIG. 5A-F, BT503 attenuates albuminuria, podocyte injury, and the dexamethasone dose in the setting of LPS treatment. FIG. 5A shows a representative schematic of the LPS proteinuric model. FIG. 5B shows representative Urine albumin:creatinine ratio in DMSO- and BT503-treated mice in the setting of LPS versus VEH administration (n=6-10, **p<0.01, ***p<0.001, Kruskal-Wallis test with Dunn's post-test). FIG. 5C shows representative Transmission Electron Microscopy showing podocyte foot processes in DMSO- and BT503-treated mice in the setting of LPS administration. Representative images from 3 independent experiments are shown. Inset shows quantification of foot process (FP) width (n=3, 10 glomeruli each, measurements per group, *p<0.05, **p<0.01, Kruskal-Wallis test with Dunn's post-test). FIG. 5D shows representative fold change in Klf15 and Wilms tumor 1 (Wt1) mRNA expression (n=6-8, *p<0.05, (Klf15); #p<0.05, ##p<0.01, (Wt1), Kruskal-Wallis test with Dunn's post-test). FIG. 5E shows representative fold change in KLF15 activity (relative to DMSO, normalized to Renilla) (n=6-12, *p<0.05, ***p<0.001, Kruskal-Wallis test with Dunn's post-test). FIG. 5F shows representative urine albumin:creatinine ratio in DMSO-, DEX-, BT503-, and combination BT503/DEX-treated mice in the setting of LPS versus VEH administration (n=5-12, *p<0.05, **p<0.01, ***p<0.001, Kruskal-Wallis test with Dunn's post-test).

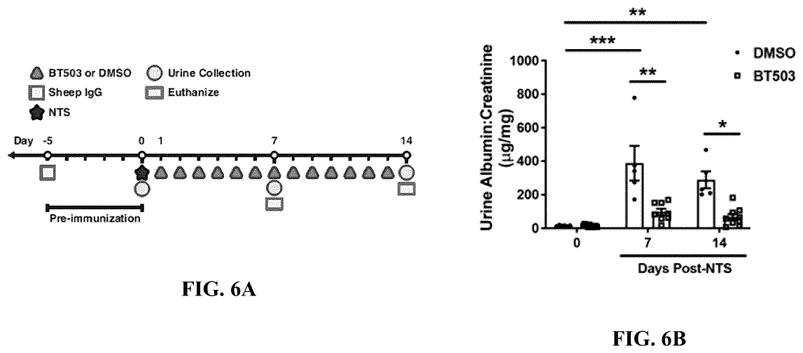

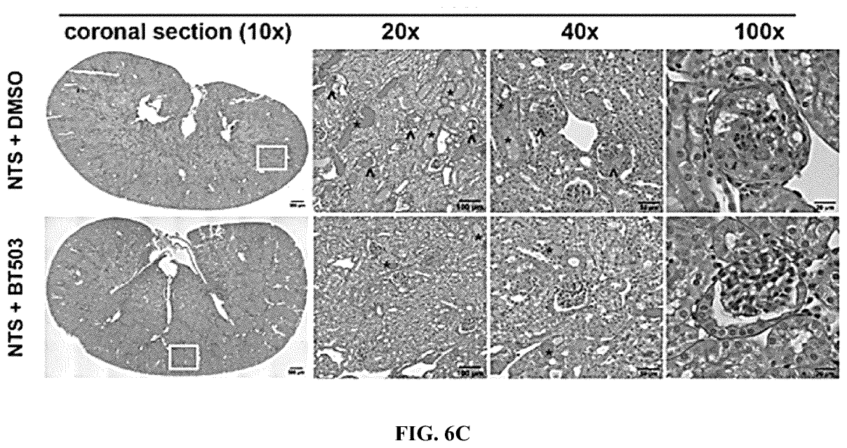

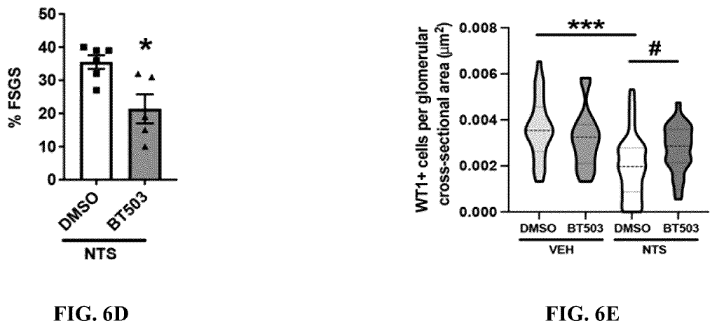

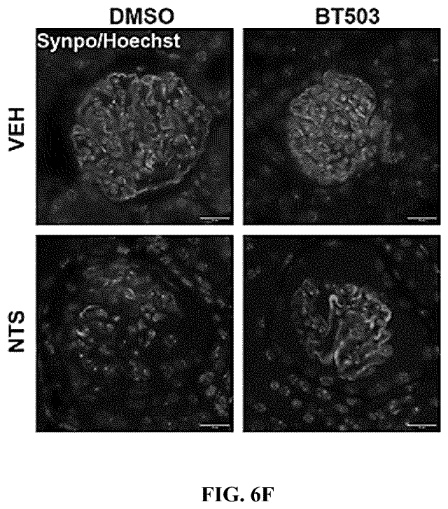

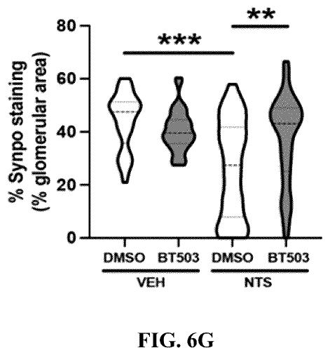

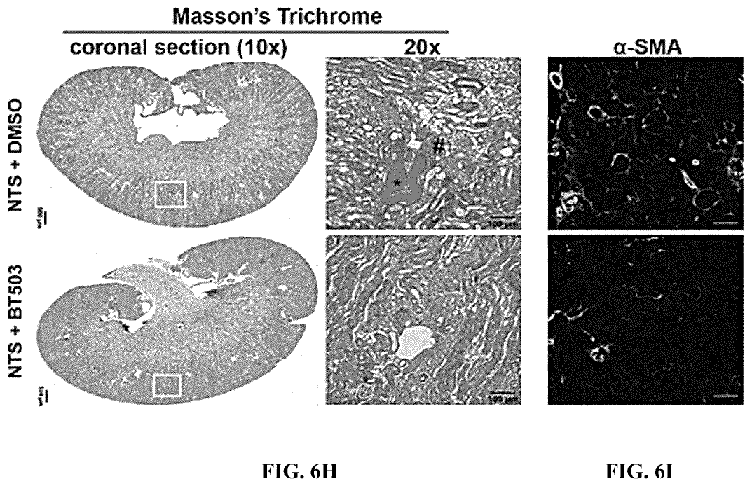

To test the therapeutic efficacy of BT503 in additional proteinuric models with progressive kidney disease, the Nephrotoxic Serum Nephritis (NTS) proteinuric model of FSGS was utilized (V. A. Rufanova et al., (2009) Kidney Int 75, 31-40) (FIG. 6A). BT503-treated mice exhibited significantly less albuminuria as compared to DMSO-treated mice at day 7 and 14 post-NTS treatment (FIG. 6B). In addition, BT503 significantly reduced the amount of glomerular injury (% of glomeruli with FSGS, extracapillary proliferation) and proteinaceous casts and restored the podocyte number (WT1+ podocytes per glomerular cross-sectional area) as compared to DMSO treatment (FIG. 6C-E). Furthermore, glomerular synaptopodin expression was restored in BT503-treated mice as compared to DMSO-treated mice (FIG. 6F and FIG. 6G). BT503-treated mice also exhibited less interstitial fibrosis as compared to DMSO-treated mice using masson's trichrome and α-SMA staining (FIG. 6H and FIG. 6I).

Referring to FIG. 6A-I, BT503 attenuates albuminuria, podocyte injury, and glomerulosclerosis post-NTS treatment. FIG. 6A shows a representative schematic of the NTS nephritis proteinuric model. FIG. 6B shows representative urine albumin:creatinine ratio in DMSO-, BT503-treated mice post-nephrotoxic serum (NTS) versus IgG administration (n=5-10, *p<0.05, **p<0.01, ***p<0.001, Kruskal-Wallis test with Dunn's post-test). FIG. 6C shows representative Periodic Acid-Schiff staining. Representative images are shown at 10×, 20×, 40×, and 100×. White box shows areas of higher magnification. Arrowheads indicate sclerotic glomeruli. * indicates protein casts. FIG. 6D shows representative % of glomeruli with FSGS lesions (n=5-6, *p<0.05, Mann-Whitney Test). FIG. 6E shows representative Podocyte number determined by WT1+ cells per glomerular cross-sectional area (n=4 mice, n=30 glomeruli per mouse, ***p<0.001, Kruskal-Wallis test with Dunn's post-test; #p<0.05, Mann-Whitney Test). FIG. 6F shows representative immunostaining for Synaptopodin (Synpo) and Hoechst. Representative images from three different experiments are shown. FIG. 6G shows representative quantification of glomerular Synaptopodin expression (% area stained) (n=3, 30 glomeruli per mouse, **p<0.01, ***p<0.001, Kruskal-Wallis test with Dunn's post-test). FIG. 6H shows representative Masson's Trichrome staining of kidney cortex. Representative images are shown at 1Ox and 20×. White box shows areas of higher magnification. #indicates areas of interstitial fibrosis. *indicates protein casts. FIG. 6I shows representative immunostaining for α-SMA. Representative images from three different experiments are shown.

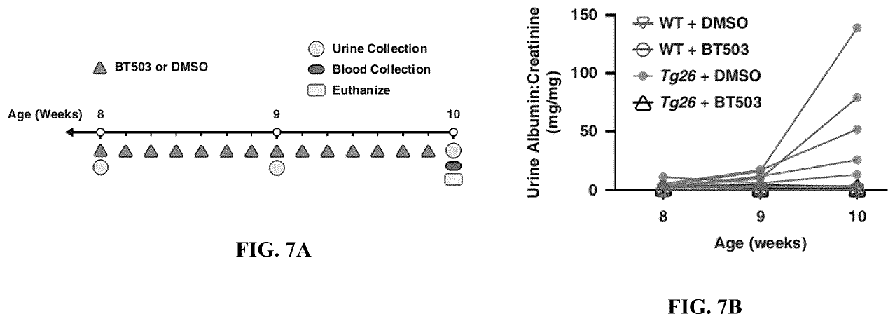

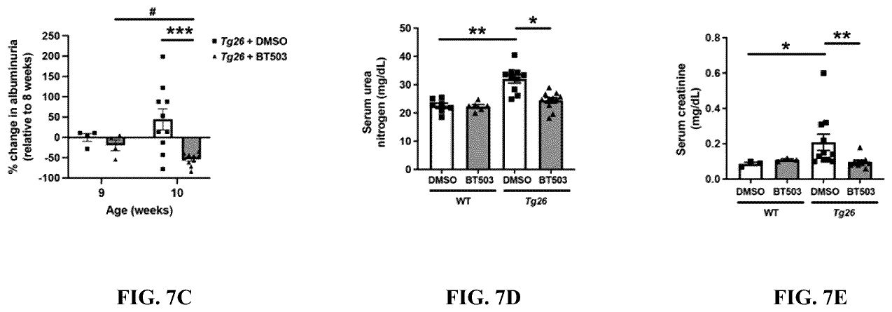

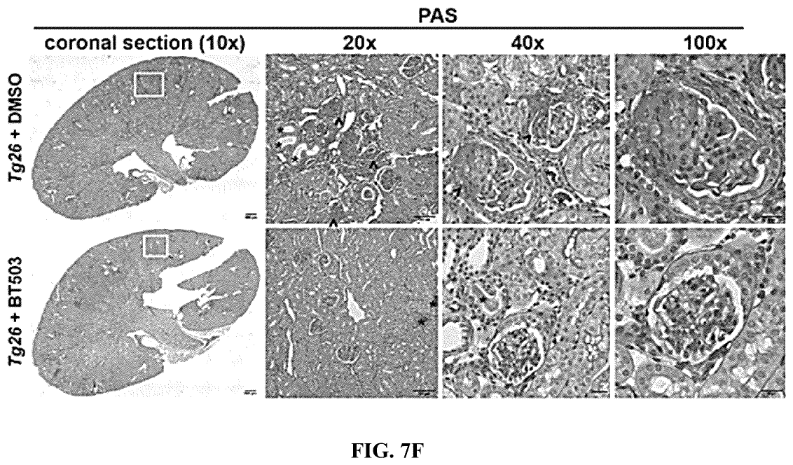

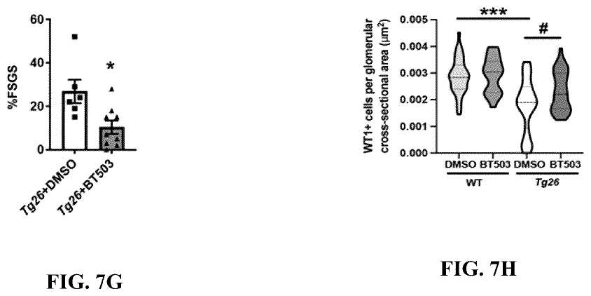

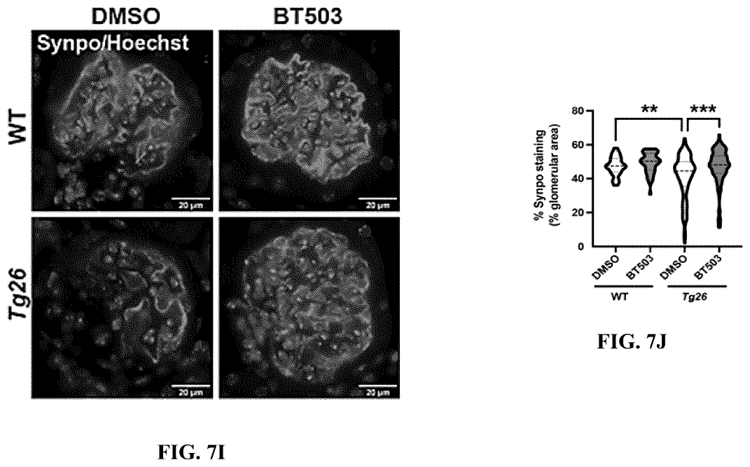

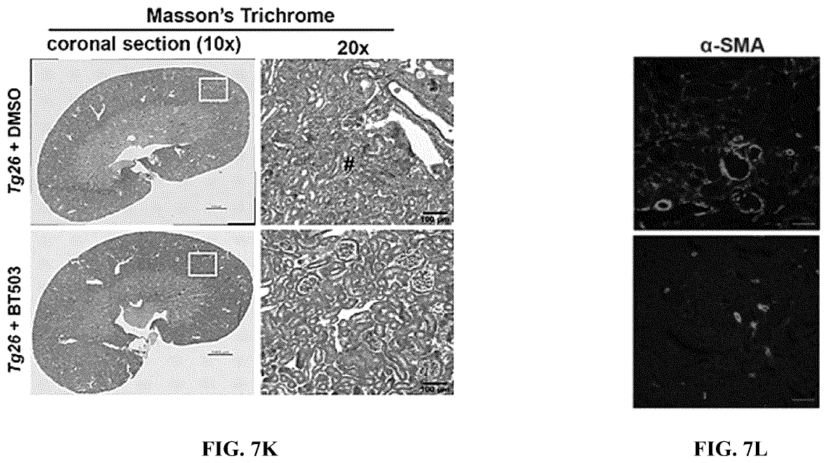

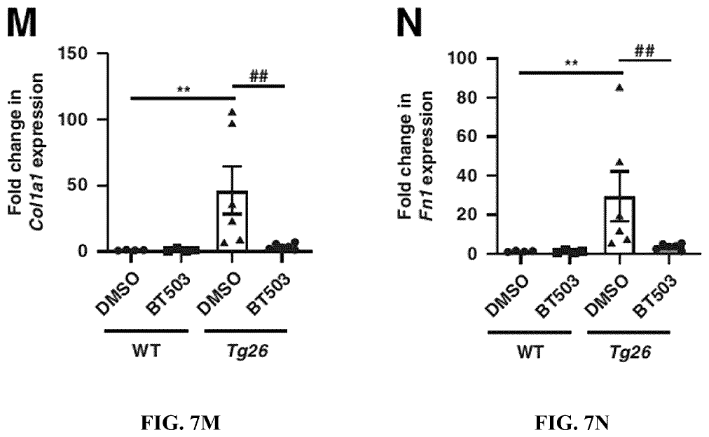

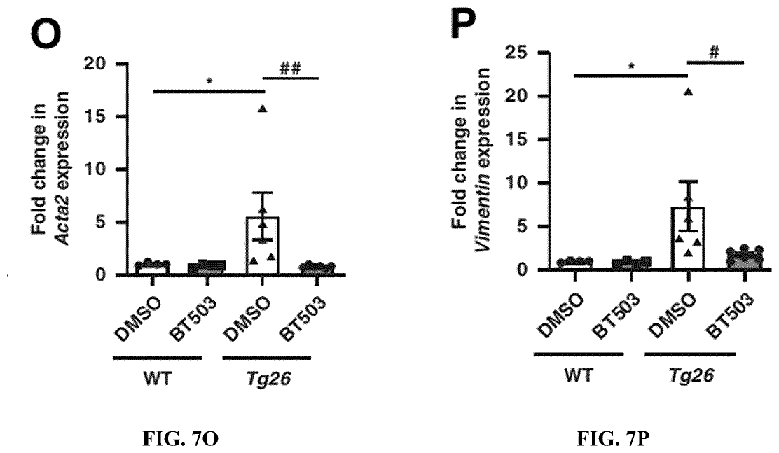

To further validate these findings, the efficacy of BT503 was tested in the HIV-1 transgenic (Tg26) mice, a model of progressive podocyte loss, severe proteinuria, and collapsing FSGS (P. Dickie et al., (1991) Virology 185, 109-119). Since significant albuminuria begins at 3-4 weeks and progresses to FSGS lesions by 7-8 weeks of age in the Tg26 mice, whether treatment with BT503 can mitigate the continued rise of albuminuria and development of FSGS lesions in these mice was tested. Initially, Tg26 mice with significant albuminuria (>1 mg·mg urine albumin/creatine) at 8 weeks of age were selected to ensure likelihood of progression of kidney disease. Subsequently, these mice were treated with BT503 or VEH daily for a 14-day period (FIG. 7A). It was observed that the BT503-treated Tg26 mice significantly mitigated the increase in albuminuria and improved kidney function (lower serum urea nitrogen and creatinine) as compared to DMSO-treatment (FIG. 7B-E). PAS staining confirmed that BT503 significantly reduced the % of glomeruli with focal segmental and global GS lesions, and preserved podocyte number (WT1+ podocytes per glomerular cross-sectional area) as compared to DMSO treatment (FIG. 7F-H). Glomerular synaptopodin expression was also restored in BT503-treated Tg26 mice as compared to DMSO-treated Tg26 mice (FIG. 7I and FIG. 7J). Similar to the NTS model, treatment with BT503 reduced interstitial fibrosis as compared to DMSO (FIG. 7K and FIG. 7L). These findings were confirmed by measuring cortical mRNA expression of pro-fibrotic markers (Col1a1, Fibronectin 1 (Fn1), Acta2, and Vimentin) (FIG. 7M-P). Collectively, these findings suggest that BT503 ameliorates podocyte injury and loss leading to a reduction in albuminuria and glomerulosclerosis with an improvement in kidney function in a murine model of progressive kidney disease.

Referring to FIG. 7A-P, BT503 attenuates albuminuria, podocyte injury, and glomerulosclerosis in Tg26 mice. FIG. 7C shows representative % change in urine albumin:creatinine (albuminuria) in Tg26 mice treated with DMSO or BT503 for 2 weeks (relative to albuminuria at 8 weeks) (n=8, ***p<0.001, 2-way ANOVA; #p<0.05, Mann-Whitney Test). FIG. 7D shows representative serum urea nitrogen and FIG. 7E shows representative serum creatinine (n=9-10, *p<0.05, **p<0.01, Kruskal-Wallis test with Dunn's post-test). FIG. 7F shows representative periodic Acid-Schiff staining of kidney cortex. Representative images are shown at 10× and 20×. White box shows areas of higher magnification. Arrowheads indicate sclerotic glomeruli. *indicates protein casts. FIG. 7G shows representative % of glomeruli with FSGS lesions (n=6-9, *p<0.05, Mann-Whitney Test). FIG. 7H shows representative podocyte number determined by WT1+ cells per glomerular cross-sectional area (n=4 mice, 30 glomeruli per mouse, ***p<0.001, Kruskal-Wallis test with Dunn's post-test; #p<0.05, Mann-Whitney Test). FIG. 7I shows representative immunostaining for Synaptopodin (Synpo) and Hoechst. Representative images from three different experiments are shown. FIG. 7J shows representative quantification of glomerular Synaptopodin expression (% area stained) (n=3, 30 glomeruli per mouse, **p<0.01, ***p<0.001, Kruskal-Wallis test with Dunn's post-test). FIG. 7K shows representative Masson's Trichrome staining of kidney cortex. Representative images are shown at 10× and 20×. White box shows areas of higher magnification. #indicates areas of interstitial fibrosis. FIG. 7L shows representative immunostaining for α-SMA. Representative images from three different experiments are shown.

### 6. BT503 Attenuates Pro-Inflammatory Pathways Under Cell Stress

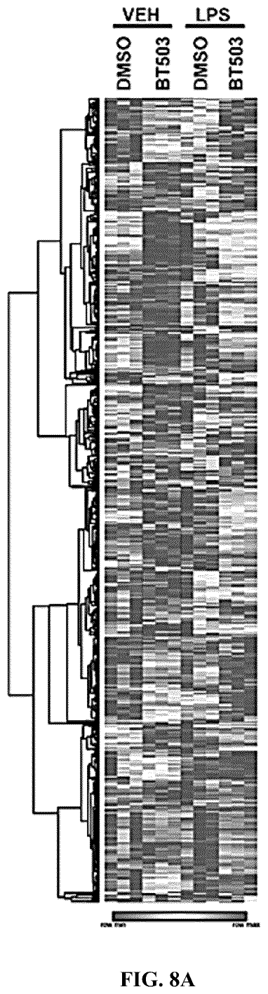

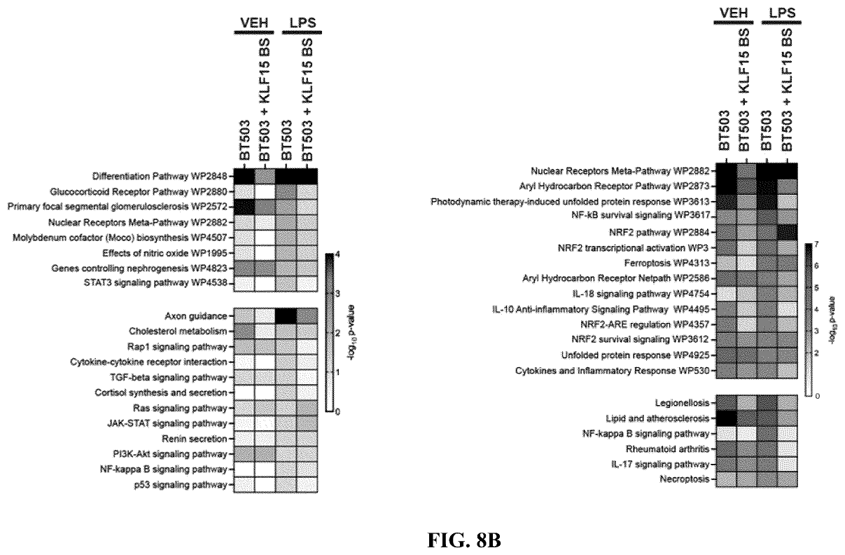

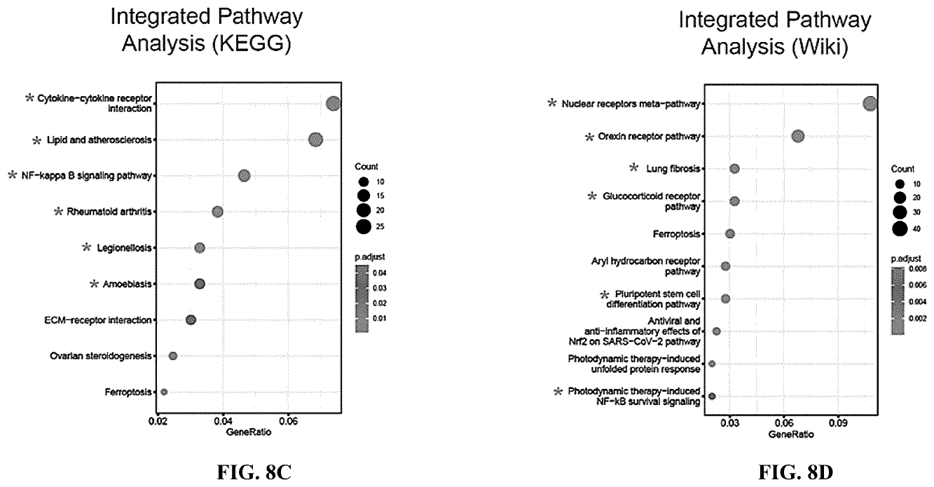

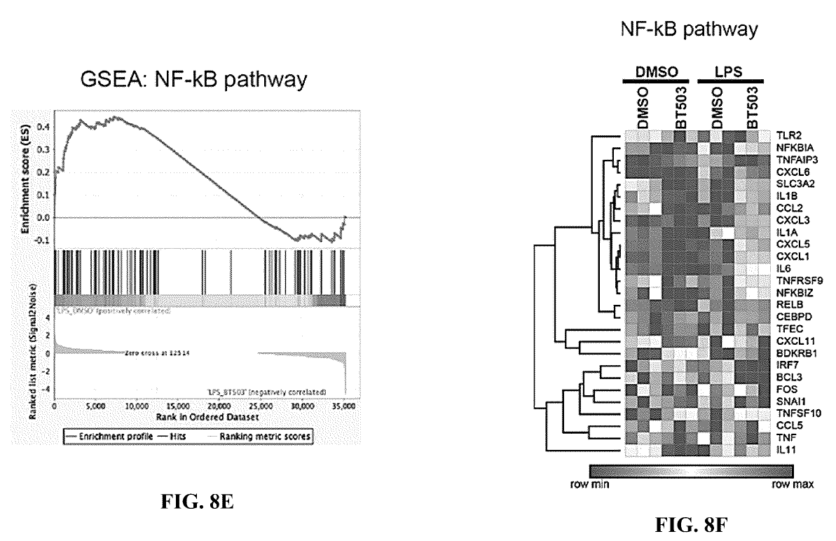

To investigate the potential pathways by which BT503-KLF15 restores podocyte injury, RNA-sequencing was conducted in differentiated human podocytes treated with and without BT503 in the setting of LPS treatment. Initially, the expression profiles of the top 600 differentially expressed genes between all four groups were visualized (FIG. 8A). The ranked upregulated and downregulated differentially expressed genes (DEGs) between the BT503 as compared in the DMSO with and without LPS are provided in supporting tables (Table 4 and Table 5). An enrichment analysis was conducted by applying the tool Enrichr (Chen et al. (2013) BMC Bioinformatics 14: 128) to the DEGs (upregulated and downregulated) in the BT503 groups as compared to DMSO (+/−LPS) using the gene set libraries: WikiPathways (Pico et al. (2008) PLoS Biol 6: e184; Slenter et al. (2018) Nucleic Acids Res 46: D661-d667) and KEGG pathways (M. Kanehisa, S. Goto, (2000) Nucleic Acids Res 28, 27-30). Upregulated DEGs post-BT503 treatment revealed a significant increase in pathways involved in cell differentiation, axon guidance, and GC signaling (FIG. 8B). In comparison, there was an enrichment of nuclear receptors meta-pathway, NF-κB survival signaling pathway, and other inflammatory pathways in the downregulated DEGs (FIG. 8B). By crossmatching these DEGs with predicted KLF15 binding sites in their promoter demonstrates an enrichment of these pathways that might be directly regulated by KLF15, further confirming that the salutary effects of BT503 are in part mediated by KLF15 (FIG. 8B).

TABLE 4

| Gene Symbol | Description |
|---|---|
| LDLR | low density lipoprotein receptor |
| PPME1 | protein phosphatase methylesterase 1 |
| SCD | stearoyl-CoA desaturase |
| SLC22A5 | solute carrier family 22 member 5 |
| TINAGL1 | tubulointerstitial nephritis antigen like 1 |
| CCN2 | cellular communication network factor 2 |
| COL8A1 | collagen type VIII alpha 1 chain |
| COL11AI | collagen type XI alpha 1 chain |
| FASN | fatty acid synthase |
| LTBP2 | latent transforming growth factor beta binding protein 2 |
| LTBP3 | latent transforming growth factor beta binding protein 3 |
| TENM2 | teneurin transmembrane protein 2 |
| PLK2 | polo like kinase 2 |
| F8A2 | coagulation factor VIII associated 2 |
| G0S2 | G0/G1 switch 2 |
| ENC1 | ectodermal-neural cortex 1 |
| DCDC2 | doublecortin domain containing 2 |
| MAMDC2 | MAM domain containing 2 |
| SEMA7A | semaphorin 7A (John Milton Hagen blood group) |
| AHNAK | AHNAK nucleoprotein |
| OXTR | oxytocin receptor |
| KRT80 | keratin 80 |
| IRS1 | insulin receptor substrate 1 |
| ITGA3 | integrin subunit alpha 3 |
| CAV1 | caveolin 1 |
| GAS6 | growth arrest specific 6 |
| FABP5P7 | fatty acid binding protein 5 pseudogene 7 |
| FJX1 | four-jointed box kinase 1 |
| HAPLN1 | hyaluronan and proteoglycan link protein 1 |
| LSS | lanosterol synthase |
| ADAMTS3 | ADAM metallopeptidase with thrombospondin type 1 motif 3 |
| NPR3 | natriuretic peptide receptor 3 |
| NPRL3 | NPR3 like, GATOR1 complex subunit |
| AXL | AXL receptor tyrosine kinase |
| HS3ST3A1 | heparan sulfate-glucosamine 3-sulfotransferase 3A1 |
| ANXA3 | annexin A3 |
| GFRA1 | GDNF family receptor alpha 1 |
| COL7A1 | collagen type VII alpha 1 chain |
| LOXL1 | lysyl oxidase like 1 |
| MTCL1 | microtubule crosslinking factor 1 |
| EFEMP1 | EGF containing fibulin extracellular matrix protein 1 |
| F2R | coagulation factor II thrombin receptor |
| KRT7 | keratin 7 |
| AGRN | agrin |
| WNT5B | Wnt family member 5B |
| TGM2 | transglutaminase 2 |
| CD70 | CD70 molecule |
| ELFN2 | extracellular leucine rich repeat and fibronectin type III |
| | domain containing 2 |
| ADAMTSL1 | ADAMTS like 1 |
| NIBAN2 | niban apoptosis regulator 2 |
| NR2F2 | nuclear receptor subfamily 2 group F member 2 |
| NUAK2 | NUAK family kinase 2 |
| SCRIB | scribble planar cell polarity protein |
| SOX9 | SRY-box transcription factor 9 |
| ADAMTS1 | ADAM metallopeptidase with thrombospondin type 1 motif 1 |
| KAZN | kazrin, periplakin interacting protein |
| ANKRD33B | ankyrin repeat domain 33B |
| PDE1C | phosphodiesterase 1C |
| WNK4 | WNK lysine deficient protein kinase 4 |
| MVD | mevalonate diphosphate decarboxylase |
| LFNG | LFNG O-fucosylpeptide 3-beta-N-acetylglucosaminyltransferase |
| CTIF | cap binding complex dependent translation initiation factor |
| SEMA3F | semaphorin 3F |
| S1PR1 | sphingosine-1-phosphate receptor 1 |
| CHTF18 | chromosome transmission fidelity factor 18 |
| RPS6KA4 | ribosomal protein S6 kinase A4 |
| IGDCC4 | immunoglobulin superfamily DCC subclass member 4 |
| FOXD1 | forkhead box D1 |
| SREBF1 | sterol regulatory element binding transcription factor 1 |
| INSIG1 | insulin induced gene 1 |
| PDLIM2 | PDZ and LIM domain 2 |
| ITGB3 | integrin subunit beta 3 |
| TBC1D2 | TBC1 domain family member 2 |
| DHCR7 | 7-dehydrocholesterol reductase |
| S100A10 | S100 calcium binding protein A10 |
| NAT14 | N-acetyltransferase 14 (putative) |
| MFSD3 | major facilitator superfamily domain containing 3 |
| SPNS2 | SPNS lysolipid transporter 2, sphingosine-1-phosphate |
| ANO8 | anoctamin 8 |
| RAPGEF3 | Rap guanine nucleotide exchange factor 3 |
| SEMA3B | semaphorin 3B |
| BCL2L1 | BCL2 like 1 |
| FOXC2 | forkhead box C2 |
| NSUN5P1 | NSUN5 pseudogene 1 |
| PEAR1 | platelet endothelial aggregation receptor 1 |
| MAN2C1 | mannosidase alpha class 2C member 1 |
| PDGFB | platelet derived growth factor subunit B |
| EPPK1 | epiplakin 1 |
| CGNL1 | cingulin like 1 |
| SH2D5 | SH2 domain containing 5 |
| AP1M2 | adaptor related protein complex 1 subunit mu 2 |
| COL27A1 | collagen type XXVII alpha 1 chain |
| FAT3 | FAT atypical cadherin 3 |
| SYNPO | synaptopodin |
| EPS8L2 | EPS8 like 2 |
| CCBE1 | collagen and calcium binding EGF domains 1 |
| MACROD1 | mono-ADP ribosylhydrolase 1 |
| SHISA9 | shisa family member 9 |
| TRIM6 | tripartite motif containing 6 |
| ARHGAP23 | Rho GTPase activating protein 23 |
| LAMB3 | laminin subunit beta 3 |
| SLX1B | SLX1 homolog B, structure-specific endonuclease subunit |
| MEST | mesoderm specific transcript |
| C6orf132 | chromosome 6 open reading frame 132 |
| SYTL1 | synaptotagmin like 1 |
| GDF6 | growth differentiation factor 6 |
| TRPC4 | transient receptor potential cation channel subfamily C member 4 |
| PGM5P2 | phosphoglucomutase 5 pseudogene 2 |
| PAPLN | papilin, proteoglycan like sulfated glycoprotein |
| CCDC61 | coiled-coil domain containing 61 |
| HRNR | hornerin |
| KCNIP1 | potassium voltage-gated channel interacting protein 1 |
| ST6GAL2 | ST6 beta-galactoside alpha-2,6-sialyltransferase 2 |
| KIRREL3 | kirre like nephrin family adhesion molecule 3 |
| ADAMTS6 | ADAM metallopeptidase with thrombospondin type 1 motif 6 |
| MN1 | MN1 proto-oncogene, transcriptional regulator |
| SPEG | striated muscle enriched protein kinase |
| ABCC3 | ATP binding cassette subfamily C member 3 |
| CPED1 | cadherin like and PC-esterase domain containing 1 |
| CPNE7 | copine 7 |
| RRS1 | ribosome biogenesis regulator 1 homolog |
| AMZ1 | archaelysin family metallopeptidase 1 |
| GALK1 | galactokinase 1 |
| FBXL6 | F-box and leucine rich repeat protein 6 |
| LSM7 | LSM7 homolog, U6 small nuclear RNA and mRNA degradation |
| | associated |
| ACCS | 1-aminocyclopropane-1-carboxylate synthase homolog (inactive) |
| MXRA8 | matrix remodeling associated 8 |
| IER5L | immediate early response 5 like |
| NBEAL2 | neurobeachin like 2 |
| NAV3 | neuron navigator 3 |
| TLL1 | tolloid like 1 |
| SLC26A1 | solute carrier family 26 member 1 |
| ARHGAP22 | Rho GTPase activating protein 22 |
| EMP1 | epithelial membrane protein 1 |
| AMDHD2 | amidohydrolase domain containing 2 |
| RARRES2 | retinoic acid receptor responder 2 |
| HYI | hydroxypyruvate isomerase (putative) |
| LIPG | lipase G, endothelial type |
| TNFRSF6B | TNF receptor superfamily member 6b |
| KCNIP3 | potassium voltage-gated channel interacting protein 3 |
| JPH2 | junctophilin 2 |
| CDH4 | cadherin 4 |
| CCDC183 | coiled-coil domain containing 183 |
| SFN | stratifin |
| REXO2 | RNA exonuclease 2 |
| DNPH1 | 2′-deoxynucleoside 5'-phosphate N-hydrolase 1 |
| DKK1 | dickkopf WNT signaling pathway inhibitor 1 |
| WNT2B | Wnt family member 2B |
| HLX | H2.0 like homeobox |
| SLITRK5 | SLIT and NTRK like family member 5 |
| BMP4 | bone morphogenetic protein 4 |
| TBX1 | T-box transcription factor 1 |
| ACADS | acyl-CoA dehydrogenase short chain |
| ID1 | inhibitor of DNA binding 1 |
| S100A2 | S100 calcium binding protein A2 |
| CDHR2 | cadherin related family member 2 |
| MIR1915HG | MIR1915 host gene |
| IL7R | interleukin 7 receptor |
| FBXW12 | F-box and WD repeat domain containing 12 |
| CSPG4P11 | chondroitin sulfate proteoglycan 4 pseudogene 11 |
| CKMT2 | creatine kinase, mitochondrial 2 |
| XDH | xanthine dehydrogenase |
| PGGHG | protein-glucosylgalactosylhydroxylysine glucosidase |
| PIF1 | PIF1 5′-to-3′ DNA helicase |
| STX1B | syntaxin 1B |
| CHST7 | carbohydrate sulfotransferase 7 |
| SYCE1L | synaptonemal complex central element protein 1 like |
| SORCS2 | sortilin related VPS10 domain containing receptor 2 |
| TSPAN18 | tetraspanin 18 |
| SPONI | spondin 1 |
| RASA4B | RAS p21 protein activator 4B |
| LHPP | phospholysine phosphohistidine inorganic pyrophosphate phosphatase |
| KIF12 | kinesin family member 12 |
| PCSK9 | proprotein convertase subtilisin/kexin type 9 |
| KCNN4 | potassium calcium-activated channel subfamily N member 4 |
| SLC35F3 | solute carrier family 35 member F3 |
| SHANK2 | SH3 and multiple ankyrin repeat domains 2 |
| EPN3 | epsin 3 |
| S100A3 | S100 calcium binding protein A3 |
| SLC52A1 | solute carrier family 52 member 1 |
| PAK3 | p21 (RAC1) activated kinase 3 |
| ARHGEF7 | Rho guanine nucleotide exchange factor 7 |
| USP43 | ubiquitin specific peptidase 43 |
| FLG2 | filaggrin 2 |
| ACBD4 | acyl-CoA binding domain containing 4 |
| RTEL1-TNFRSF6B | RTEL1-TNFRSF6B readthrough (NMD candidate) |
| COL13AI | collagen type XIII alpha 1 chain |
| SLC16A8 | solute carrier family 16 member 8 |
| ZRSR2P1 | ZRSR2 pseudogene 1 |
| CSDC2 | cold shock domain containing C2 |
| HSD11B1L | hydroxysteroid 11-beta dehydrogenase 1 like |
| PSMC1P1 | proteasome 26S subunit, ATPase 1 pseudogene 1 |
| P2RY2 | purinergic receptor P2Y2 |
| METTL27 | methyltransferase like 27 |
| NTHL1 | nth like DNA glycosylase 1 |
| IKBKGP1 | inhibitor of nuclear factor kappa B kinase subunit gamma pseudogene 1 |
| ITGA2B | integrin subunit alpha 2b |
| FGFR4 | fibroblast growth factor receptor 4 |
| SNAI3 | snail family transcriptional repressor 3 |
| BAIAP2L2 | BAR/IMD domain containing adaptor protein 2 like 2 |
| B4GALNT4 | beta-1,4-N-acetyl-galactosaminyltransferase 4 |
| DOK7 | docking protein 7 |
| WT1 | WT1 transcription factor |
| LMF1 | lipase maturation factor 1 |
| KLF4 | KLF transcription factor 4 |
| MAMDC4 | MAM domain containing 4 |
| SH3TC2 | SH3 domain and tetratricopeptide repeats 2 |
| VSIG8 | V-set and immunoglobulin domain containing 8 |
| GPRIN2 | G protein regulated inducer of neurite outgrowth 2 |
| GPT | glutamic -- pyruvic transaminase |
| DPAGTI | dolichyl-phosphate N-acetylglucosaminephosphotransferase 1 |
| P4HA3 | prolyl 4-hydroxylase subunit alpha 3 |
| ST6GALNAC5 | ST6 N-acetylgalactosaminide alpha-2,6-sialyltransferase 5 |
| INKA1 | inka box actin regulator 1 |
| HRCT1 | histidine rich carboxyl terminus 1 |
| Clorf116 | chromosome 1 open reading frame 116 |
| CAMK2A | calcium/calmodulin dependent protein kinase II alpha |
| NEURL2 | neuralized E3 ubiquitin protein ligase 2 |
| PTRHI | peptidyl-tRNA hydrolase 1 homolog |
| GPC2 | glypican 2 |
| PABPC4L | poly(A) binding protein cytoplasmic 4 like |
| LENG1 | leukocyte receptor cluster member 1 |
| FMC1 | formation of mitochondrial complex V assembly factor 1 homolog |
| CCDC198 | coiled-coil domain containing 198 |
| RAB43 | RAB43, member RAS oncogene family |
| RASAL1 | RAS protein activator like 1 |
| EDF1 | endothelial differentiation related factor 1 |
| SSR4P1 | signal sequence receptor subunit 4 pseudogene 1 |
| MYEOV | myeloma overexpressed |
| B3GNTL1 | UDP-GlcNAc:betaGal beta-1,3-N-acetylglucosaminyltransferase like 1 |
| DMBT1 | deleted in malignant brain tumors 1 |
| CDH16 | cadherin 16 |
| ASIC2 | acid sensing ion channel subunit 2 |
| PGM5 | phosphoglucomutase 5 |
| KCNK10 | potassium two pore domain channel subfamily K member 10 |
| SMIM11B | |
| LINC00431 | long intergenic non-protein coding RNA 431 |
| FGF1 | fibroblast growth factor 1 |
| PCDHGC5 | protocadherin gamma subfamily C, 5 |
| KLHL13 | kelch like family member 13 |
| EDN1 | endothelin 1 |
| PTX3 | pentraxin 3 |
| HUNK | hormonally up-regulated Neu-associated kinase |
| HEPACAM | hepatic and glial cell adhesion molecule |
| NKAIN4 | sodium/potassium transporting ATPase interacting 4 |
| GNBIL | G protein subunit beta 1 like |
| KLHL4 | kelch like family member 4 |
| SOAT2 | sterol O-acyltransferase 2 |
| GLYCTK | glycerate kinase |
| CD163L1 | CD163 molecule like 1 |
| KMT5AP1 | KMT5A pseudogene 1 |
| ADAMTS16 | ADAM metallopeptidase with thrombospondin type 1 motif 16 |
| SBK2 | SH3 domain binding kinase family member 2 |
| NOG | noggin |
| PROM2 | prominin 2 |
| HSD17B8 | hydroxysteroid 17-beta dehydrogenase 8 |
| SOWAHD | sosondowah ankyrin repeat domain family member D |
| PCDHGA6 | protocadherin gamma subfamily A, 6 |
| DNAJB13 | DnaJ heat shock protein family (Hsp40) member B13 |
| KAAG1 | kidney associated DCDC2 antisense RNA 1 |
| DYNLT4 | dynein light chain Tctex-type 4 |
| HSPD1P11 | heat shock protein family D (Hsp60) member 1 pseudogene 11 |
| MPP3 | MAGUK p55 scaffold protein 3 |
| KIF17 | kinesin family member 17 |
| CRYBG2 | crystallin beta-gamma domain containing 2 |
| DIRAS1 | DIRAS family GTPase 1 |
| PTAFR | platelet activating factor receptor |
| GPR39 | G protein-coupled receptor 39 |
| ACHE | acetylcholinesterase (Cartwright blood group) |
| MMP23B | matrix metallopeptidase 23B |
| PHYHIP | phytanoyl-CoA 2-hydroxylase interacting protein |
| DERL3 | derlin 3 |
| FKBP2 | FKBP prolyl isomerase 2 |
| NRG2 | neuregulin 2 |
| NNAT | neuronatin |
| USH1G | USH1 protein network component sans |
| ULK4P3 | ULK4 pseudogene 3 |
| CYP27C1 | cytochrome P450 family 27 subfamily C member 1 |
| DSTNP1 | DSTN pseudogene 1 |
| KLK10 | kallikrein related peptidase 10 |
| CEACAM20 | CEA cell adhesion molecule 20 |
| FRG1JP | FSHD region gene 1 family member J, pseudogene |
| DCST1 | DC-STAMP domain containing 1 |
| PLTP | phospholipid transfer protein |
| KCNJ5-AS1 | KCNJ5 antisense RNA 1 |
| APLN | apelin |
| CHAD | chondroadherin |
| MATN1 | matrilin 1 |
| BGLAP | bone gamma-carboxyglutamate protein |
| RIMBP3B | RIMS binding protein 3B |
| MAMSTR | MEF2 activating motif and SAP domain containing |
| | transcriptional regulator |
| INSYN2B | inhibitory synaptic factor family member 2B |
| NT5E | 5′-nucleotidase ecto |
| PLAU | plasminogen activator, urokinase |
| IGFBP3 | insulin like growth factor binding protein 3 |
| NRP2 | neuropilin 2 |
| NELL2 | neural EGFL like 2 |
| DGKA | diacylglycerol kinase alpha |
| CAVIN2 | caveolae associated protein 2 |
| KCNH1 | potassium voltage-gated channel subfamily H member 1 |
| PTPRB | protein tyrosine phosphatase receptor type B |
| CCND1 | cyclin D1 |
| UCP2 | uncoupling protein 2 |
| HMGA2 | high mobility group AT-hook 2 |
| NFE2L3 | NFE2 like bZIP transcription factor 3 |
| CAPN2 | calpain 2 |
| HEG1 | heart development protein with EGF like domains 1 |
| DOCK4 | dedicator of cytokinesis 4 |
| RIN2 | Ras and Rab interactor 2 |
| KLF6 | KLF transcription factor 6 |
| SEMA3C | semaphorin 3C |
| SEMA5A | semaphorin 5A |
| THSD4 | thrombospondin type 1 domain containing 4 |
| CD24 | CD24 molecule |
| MYLK | myosin light chain kinase |
| AMOTL2 | angiomotin like 2 |
| LMO7 | LIM domain 7 |
| LAYN | layilin |
| SFXN2 | sideroflexin 2 |
| NRXN3 | neurexin 3 |
| CLDN4 | claudin 4 |
| NEFL | neurofilament light chain |
| AOX1 | aldehyde oxidase 1 |
| KIF20A | kinesin family member 20A |
| PRSS23 | serine protease 23 |
| DUXAP9 | double homeobox A pseudogene 9 |
| TMSB4X | thymosin beta 4 X-linked |
| TJP2 | tight junction protein 2 |
| FGF5 | fibroblast growth factor 5 |
| PDGFC | platelet derived growth factor C |
| PCDHGC3 | protocadherin gamma subfamily C, 3 |
| TNS3 | tensin 3 |
| KRT18 | keratin 18 |
| CPA4 | carboxypeptidase A4 |
| SLC37A4 | solute carrier family 37 member 4 |
| ANXA2 | annexin A2 |
| PODXL | podocalyxin like |
| FAM83D | family with sequence similarity 83 member D |
| NCKAP5 | NCK associated protein 5 |
| NMT2 | N-myristoyltransferase 2 |
| SNAPC1 | small nuclear RNA activating complex polypeptide 1 |
| ABTB3 | ankyrin repeat and BTB domain containing 3 |
| TSPAN14 | tetraspanin 14 |
| PLAUR | plasminogen activator, urokinase receptor |
| DCAF12L1 | DDB1 and CUL4 associated factor 12 like 1 |
| C2CD3 | C2 domain containing 3 centriole elongation regulator |
| POU2F2 | POU class 2 homeobox 2 |
| DRAXIN | dorsal inhibitory axon guidance protein |
| HLA-DOA | major histocompatibility complex, class II, DO alpha |
| NPNT | nephronectin |
| IP6K3 | inositol hexakisphosphate kinase 3 |
| BHLHE40 | basic helix-loop-helix family member e40 |
| FRMD5 | FERM domain containing 5 |
| EZR | ezrin |
| SERPINE1 | serpin family E member 1 |
| CHRDL1 | chordin like 1 |
| ATP8B1 | ATPase phospholipid transporting 8B1 |
| TENM4 | teneurin transmembrane protein 4 |
| P3H2 | prolyl 3-hydroxylase 2 |
| SRSF2 | serine and arginine rich splicing factor 2 |
| ADGRG1 | adhesion G protein-coupled receptor G1 |
| NIPAL4 | NIPA like domain containing 4 |
| NABP1 | nucleic acid binding protein 1 |
| MEGF9 | multiple EGF like domains 9 |
| MTMR10 | myotubularin related protein 10 |
| BCAR3 | BCAR3 adaptor protein, NSP family member |
| TRERF1 | transcriptional regulating factor 1 |
| RPL21 | ribosomal protein L21 |
| SH3RF2 | SH3 domain containing ring finger 2 |
| BNC1 | basonuclin zinc finger protein 1 |
| ABLIM1 | actin binding LIM protein 1 |
| CMBL | carboxymethylenebutenolidase homolog |
| CRIM1 | cysteine rich transmembrane BMP regulator 1 |
| CD274 | CD274 molecule |
| NHS | NHS actin remodeling regulator |
| HAS2 | hyaluronan synthase 2 |
| ACTN4 | actinin alpha 4 |
| CLCA2 | chloride channel accessory 2 |
| ARHGAP18 | Rho GTPase activating protein 18 |
| ARSJ | arylsulfatase family member J |
| PRKX | protein kinase cAMP-dependent X-linked catalytic subunit |
| SERTAD4 | SERTA domain containing 4 |
| NME1 | NME/NM23 nucleoside diphosphate kinase 1 |
| RMRP | RNA component of mitochondrial RNA processing endoribonuclease |
| ADAMTS12 | ADAM metallopeptidase with thrombospondin type 1 motif 12 |
| DHCR24 | 24-dehydrocholesterol reductase |
| F2RL2 | coagulation factor II thrombin receptor like 2 |
| SCN2A | sodium voltage-gated channel alpha subunit 2 |
| GYPC | glycophorin C (Gerbich blood group) |
| NUAK1 | NUAK family kinase 1 |
| APBB2 | amyloid beta precursor protein binding family B member 2 |

TABLE 5

| Gene Symbol | Description |
|---|---|
| CYP1B1 | cytochrome P450 family 1 subfamily B member 1 |
| STC2 | stanniocalcin 2 |
| SLC7A11 | solute carrier family 7 member 11 |
| TIPARP | TCDD inducible poly(ADP-ribose) polymerase |
| SHISA2 | shisa family member 2 |
| HSPA1A | heat shock protein family A (Hsp70) member 1A |
| CYP1A1 | cytochrome P450 family 1 subfamily A member 1 |
| SLC7A5 | solute carrier family 7 member 5 |
| ANGPTL4 | angiopoietin like 4 |
| GREM1 | gremlin 1, DAN family BMP antagonist |
| OTUB2 | OTU deubiquitinase, ubiquitin aldehyde binding 2 |
| HSPA5 | heat shock protein family A (Hsp70) member 5 |
| DDIT3 | DNA damage inducible transcript 3 |
| HSPA1B | heat shock protein family A (Hsp70) member 1B |
| AHRR | aryl hydrocarbon receptor repressor |
| ASNS | asparagine synthetase (glutamine-hydrolyzing) |
| PLIN2 | perilipin 2 |
| ITGB1P1 | integrin subunit beta 1 pseudogene 1 |
| SQSTM1 | sequestosome 1 |
| NQO1 | NAD(P)H quinone dehydrogenase 1 |
| HSP90AA1 | heat shock protein 90 alpha family class A member 1 |
| TRIB3 | tribbles pseudokinase 3 |
| FTL | ferritin light chain |
| HSPH1 | heat shock protein family H (Hsp110) member 1 |
| ASB2 | ankyrin repeat and SOCS box containing 2 |
| IL6 | interleukin 6 |
| CCPG1 | cell cycle progression 1 |
| HSPA13 | heat shock protein family A (Hsp70) member 13 |
| CTH | cystathionine gamma-lyase |
| VSIG2 | V-set and immunoglobulin domain containing 2 |
| TRIM16L | tripartite motif containing 16 like (pseudogene) |
| BCL2L2- | BCL2L2-PABPN1 readthrough |
| PABPN1 | |
| GABARAPL1 | GABA type A receptor associated protein like 1 |
| MT-ND4L | mitochondrially encoded NADH:ubiquinone |
| | oxidoreductase core subunit 4L |
| ZFAND2A | zinc finger AN1-type containing 2A |
| TSC22D3 | TSC22 domain family member 3 |
| IFRD1 | interferon related developmental regulator 1 |
| CREBRF | CREB3 regulatory factor |
| ZNF473 | zinc finger protein 473 |
| FAM107B | family with sequence similarity 107 member B |
| CHAC1 | ChaC glutathione specific |
| | gamma-glutamylcyclotransferase 1 |
| DHRS3 | dehydrogenase/reductase 3 |
| OSGIN1 | oxidative stress induced growth inhibitor 1 |
| CCL2 | C-C motif chemokine ligand 2 |
| LPXN | leupaxin |
| PPP1R15A | protein phosphatase 1 regulatory subunit 15A |
| NFE2L2 | NFE2 like bZIP transcription factor 2 |
| INA | internexin neuronal intermediate filament protein alpha |
| SLU7 | SLU7 homolog, splicing factor |
| HMOX1 | heme oxygenase 1 |
| KLHL24 | kelch like family member 24 |
| LRIF1 | ligand dependent nuclear receptor interacting factor 1 |
| TRIM16 | tripartite motif containing 16 |
| KCNK3 | potassium two pore domain channel subfamily K |
| | member 3 |
| CXCL8 | C-X-C motif chemokine ligand 8 |
| CASP4 | caspase 4 |
| CENPS-CORT | CENPS-CORT readthrough |
| ADGRF1 | adhesion G protein-coupled receptor F1 |
| SAA2 | serum amyloid A2 |
| DLX2 | distal-less homeobox 2 |
| IGF2 | insulin like growth factor 2 |
| ARRDC4 | arrestin domain containing 4 |
| CENPQ | centromere protein Q |
| H2BC12 | H2B clustered histone 12 |
| QRICH2 | glutamine rich 2 |
| SC5D | sterol-C5-desaturase |
| LUM | lumican |
| MIA2 | MIA SH3 domain ER export factor 2 |
| CYTIP | cytohesin 1 interacting protein |
| ZNF511- | ZNF511-PRAP1 readthrough |
| PRAP1 | |
| ZNF273 | zinc finger protein 273 |
| GFPT2 | glutamine-fructose-6-phosphate transaminase 2 |
| CHRM4 | cholinergic receptor muscarinic 4 |
| SPX | spexin hormone |
| CXCL1 | C-X-C motif chemokine ligand 1 |
| TRIM36 | tripartite motif containing 36 |
| IL1A | interleukin 1 alpha |
| CLEC4E | C-type lectin domain family 4 member E |
| ATG4A | autophagy related 4A cysteine peptidase |
| FAM9C | family with sequence similarity 9 member C |
| CPEB3 | cytoplasmic polyadenylation element binding protein 3 |
| ZNF267 | zinc finger protein 267 |
| TIGD7 | tigger transposable element derived 7 |
| ZNF823 | zinc finger protein 823 |
| PIR | pirin |
| SH3GL2 | SH3 domain containing GRB2 like 2, endophilin A1 |
| ZNF724 | zinc finger protein 724 |
| HSPE1-MOB4 | HSPE1-MOB4 readthrough |
| RADX | RPA1 related single stranded DNA binding protein, |
| | X-linked |
| SNAI1 | snail family transcriptional repressor 1 |
| LEKR1 | leucine, glutamate and lysine rich 1 |
| H4C9 | H4 clustered histone 9 |
| FZD3 | frizzled class receptor 3 |
| FZD9 | frizzled class receptor 9 |
| RPL13P12 | ribosomal protein L13 pseudogene 12 |
| SYS1- | SYS1-DBNDD2 readthrough (NMD candidate) |
| DBNDD2 | |
| BMF | Bc12 modifying factor |
| CXCL11 | C-X-C motif chemokine ligand 11 |
| H3P6 | H3 histone pseudogene 6 |
| PTGES3L- | PTGES3L-AARSD1 readthrough |
| AARSD1 | |
| KRT34 | keratin 34 |
| BATF2 | basic leucine zipper ATF-like transcription factor 2 |
| CYP27B1 | cytochrome P450 family 27 subfamily B member 1 |
| MAFA | MAF bZIP transcription factor A |
| KLRG1 | killer cell lectin like receptor G1 |
| BBS12 | Bardet-Biedl syndrome 12 |
| COLQ | collagen like tail subunit of asymmetric |
| | acetylcholinesterase |
| HOXC8 | homeobox C8 |
| PDE8B | phosphodiesterase 8B |
| PRRG3 | proline rich and Gla domain 3 |
| HSPA6 | heat shock protein family A (Hsp70) member 6 |
| BCAS1 | brain enriched myelin associated protein 1 |
| LRRC49 | leucine rich repeat containing 49 |
| FAM47E- | FAM47E-STBD1 readthrough |
| STBD1 | |
| CRABP2 | cellular retinoic acid binding protein 2 |
| ZNF100 | zinc finger protein 100 |
| TMED7- | TMED7-TICAM2 readthrough |
| TICAM2 | |
| HLA-DMB | major histocompatibility complex, class II, DM beta |
| ZNF570 | zinc finger protein 570 |
| FRG1BP | FSHD region gene 1 family member B, pseudogene |
| TMEM179 | transmembrane protein 179 |
| GKAP1 | G kinase anchoring protein 1 |
| FOS | Fos proto-oncogene, AP-1 transcription factor subunit |
| ZCCHC12 | zinc finger CCHC-type containing 12 |
| ILIB | interleukin 1 beta |
| MICOS10- | MICOS10-NBL1 readthrough |
| NBL1 | |
| FMC1- | FMC1-LUC7L2 readthrough |
| LUC7L2 | |
| AOC2 | amine oxidase copper containing 2 |
| CCDC181 | coiled-coil domain containing 181 |
| GYG2P1 | glycogenin 2 pseudogene 1 |
| DNAJC27 | DnaJ heat shock protein family (Hsp40) member C27 |
| MYO7A | myosin VIIA |
| IRGM | immunity related GTPase M |
| ULBP3 | UL16 binding protein 3 |
| TMED10P1 | transmembrane p24 trafficking protein 10 |
| | pseudogene 1 |
| RND1 | Rho family GTPase 1 |
| ZNF695 | zinc finger protein 695 |
| GPR68 | G protein-coupled receptor 68 |
| RGPD6 | RANBP2 like and GRIP domain containing 6 |
| MPZL3 | myelin protein zero like 3 |
| BTBD8 | BTB domain containing 8 |
| ATP1B2 | ATPase Na+/K+ transporting subunit beta 2 |
| PLA2G4C | phospholipase A2 group IVC |
| CAB39L | calcium binding protein 39 like |
| ELAVL3 | ELAV like RNA binding protein 3 |
| SLC6A9 | solute carrier family 6 member 9 |
| CTAGE8 | CTAGE family member 8 |
| DPY19L1P1 | DPY19L1 pseudogene 1 |
| MMP16 | matrix metallopeptidase 16 |
| XKR9 | XK related 9 |
| RNASEK- | RNASEK-C17orf49 readthrough |
| C17orf49 | |
| TIGD4 | tigger transposable element derived 4 |
| ZNF214 | zinc finger protein 214 |
| IQCK | IQ motif containing K |
| SHE | Src homology 2 domain containing E |
| FGF23 | fibroblast growth factor 23 |
| CKMT1B | creatine kinase, mitochondrial 1B |
| TAS2R4 | taste 2 receptor member 4 |
| EFHD1 | EF-hand domain family member D1 |
| GAREM1 | GRB2 associated regulator of MAPK1 subtype 1 |
| STIMATE- | STIMATE-MUSTN1 readthrough |
| MUSTN1 | |
| EIF3CL | eukaryotic translation initiation factor 3 subunit C like |
| C3orf33 | chromosome 3 open reading frame 33 |
| CPA2 | carboxypeptidase A2 |
| ZFP37 | ZFP37 zinc finger protein |
| UBE2FP1 | UBE2F pseudogene 1 |
| PPM1L | protein phosphatase, Mg2+/Mn2+ dependent 1L |
| GTF2IRD2P1 | GTF2I repeat domain containing 2 pseudogene 1 |
| SMIM11 | small integral membrane protein 11 |
| SLC3A1 | solute carrier family 3 member 1 |
| SLC43A1 | solute carrier family 43 member 1 |
| HPCAL4 | hippocalcin like 4 |
| ZNF568 | zinc finger protein 568 |
| TEN1-CDK3 | TEN1-CDK3 readthrough (NMD candidate) |
| BEX2 | brain expressed X-linked 2 |
| CNTF | ciliary neurotrophic factor |
| GP1BB | glycoprotein Ib platelet subunit beta |
| OGN | osteoglycin |
| MAP2 | microtubule associated protein 2 |
| METAP2 | methionyl aminopeptidase 2 |
| INO80B-WBP1 | INO80B-WBP1 readthrough (NMD candidate) |
| EML1 | EMAP like 1 |
| ZNF836 | zinc finger protein 836 |
| ZNF43 | zinc finger protein 43 |
| RAB39B | RAB39B, member RAS oncogene family |
| C12orf50 | chromosome 12 open reading frame 50 |
| LPA | lipoprotein(a) |
| CXCL2 | C-X-C motif chemokine ligand 2 |
| SRXN1 | sulfiredoxin 1 |
| TTN | titin |
| ATF3 | activating transcription factor 3 |
| SEPHS2 | selenophosphate synthetase 2 |
| SLC3A2 | solute carrier family 3 member 2 |
| ANKRD13C | ankyrin repeat domain 13C |
| CXCL3 | C-X-C motif chemokine ligand 3 |
| TPCN1 | two pore segment channel 1 |
| KRTAP2-3 | keratin associated protein 2-3 |
| FREM2 | FRASI related extracellular matrix 2 |
| HLA-E | major histocompatibility complex, class I, E |
| MAFG | MAF bZIP transcription factor G |
| MYBL2 | MYB proto-oncogene like 2 |
| RASSF1 | Ras association domain family member 1 |
| TPRA1 | transmembrane protein adipocyte associated 1 |
| GPR153 | G protein-coupled receptor 153 |
| HDAC11 | histone deacetylase 11 |
| RHBDL3 | rhomboid like 3 |
| AGTR1 | angiotensin II receptor type 1 |
| FBN1 | fibrillin 1 |
| TBRG1 | transforming growth factor beta regulator 1 |
| NCF2 | neutrophil cytosolic factor 2 |
| PSENEN | presenilin enhancer, gamma-secretase subunit |
| GLA | galactosidase alpha |
| NAT8 | N-acetyltransferase 8 (putative) |
| DNAJB9 | DnaJ heat shock protein family (Hsp40) member B9 |
| IER3 | immediate early response 3 |
| USH2A | usherin |
| UNKL | unk like zinc finger |
| FLG | filaggrin |
| FGFR1 | fibroblast growth factor receptor 1 |
| SLC66A1 | solute carrier family 66 member 1 |
| MAP1LC3B | microtubule associated protein 1 light chain 3 beta |
| INPP5K | inositol polyphosphate-5-phosphatase K |
| SMCR8 | SMCR8-C9orf72 complex subunit |

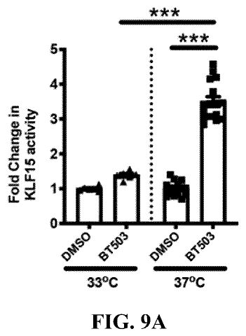

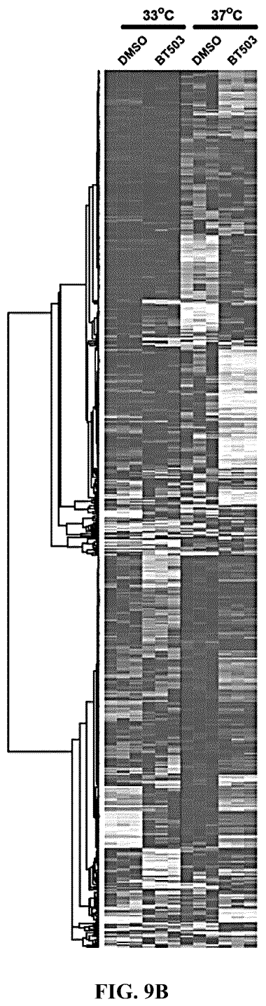

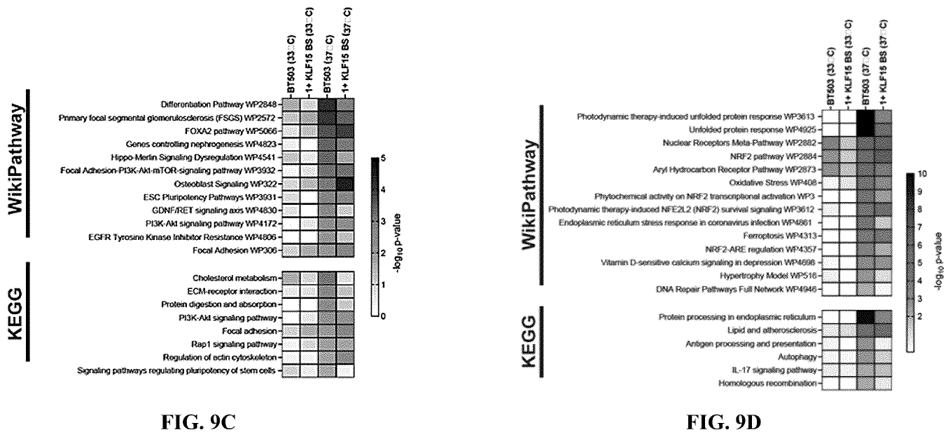

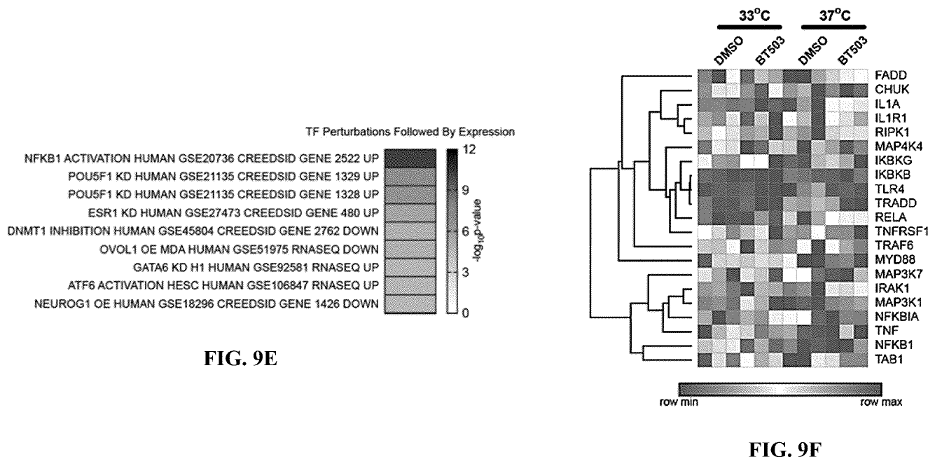

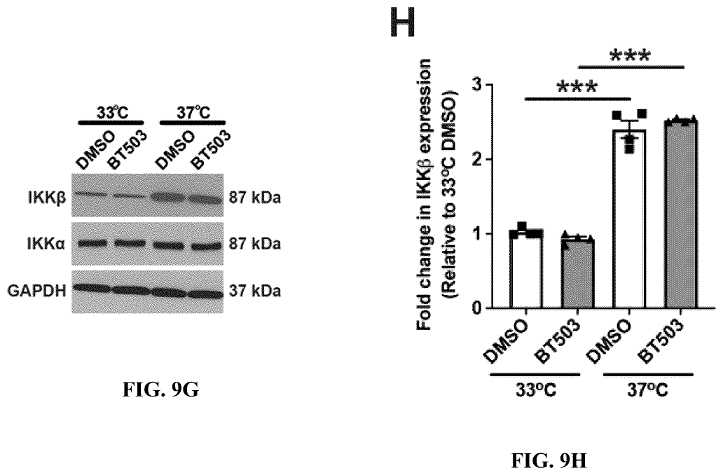

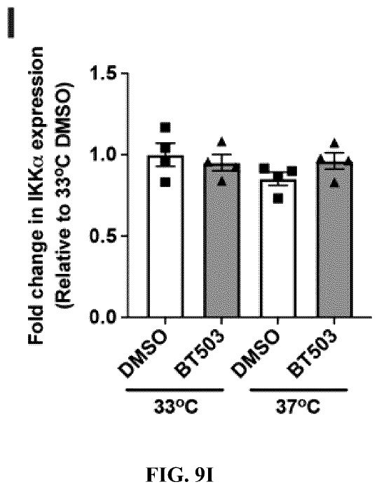

Since NF-κB signaling has been previously reported to negatively regulate KLF15 (X. Gao et al., (2011) Kidney Int 79, 987-996, Y. Lu et al., (2013) J Clin Invest 123, 4232-4241, B. Liu et al., (2018) Mol Med Rep 18, 1987-1994), an integrated pathway analysis (KEGG and WikiPathways) was conducted of both upregulated and downregulated DEGs using ClusterProfiler to validate an enrichment of several pathways involving NF-κB signaling (FIG. 8C and FIG. 8D). Gene set enrichment analysis (GSEA) with heatmap analysis of DEGs involved in NF-κB signaling also shows a downregulation with BT503 as compared to DMSO in the setting of LPS treatment (FIG. 8E and FIG. 8F). To test the potential specificity of BT503 to NF-κB signaling, the conditions were tested that enable the salutary effects of BT503. It was observed that BT503 only induces KLF15 activity under nonpermissive (37 C) conditions (i.e., differentiated podocytes) as compared to permissive (33 C) conditions (FIG. 9A). Subsequent RNA sequencing demonstrated a significant enrichment of similar pathways involving cell differentiation from the upregulated DEGs and proinflammatory pathways from the downregulated DEGs post-BT503 treatment under nonpermissive conditions (FIG. 91B-D). The ranked upregulated and downregulated differentially expressed genes (DEGs) are provided in Table 6 and Table 7, respectively. (Note: both tables illustrate BT503 vs. DMSO in nonpermissive conditions (37 C) relative to permissive conditions (33° C.)). Interestingly, in silico Chromatin Immunoprecipitation Enrichment Analysis (ChEA) (A. Lachmann et al., (2010) Bioinformatics 26, 2438-2444) demonstrated NFKB1 as the most statistically significant transcription factor with binding sites that might occupy the promoter of these DEGs (FIG. 9E). These data demonstrate that BT503 inhibits proinflammatory pathways, specifically NF-κB signaling, in human podocytes in the setting of cell stress.

TABLE 6

| Gene Symbol | Description |
|---|---|
| C3 | complement C3 |
| CPA4 | carboxypeptidase A4 |
| CDKN1A | cyclin dependent kinase inhibitor 1A |
| SERPINE1 | serpin family E member 1 |
| GDF15 | growth differentiation factor 15 |
| NT5E | 5′-nucleotidase ecto |
| LTBP2 | latent transforming growth factor beta binding protein 2 |
| LTBP3 | latent transforming growth factor beta binding protein 3 |
| PHLDA1 | pleckstrin homology like domain family A member 1 |
| PSTPIP2 | proline-serine-threonine phosphatase interacting protein 2 |
| TRIM22 | tripartite motif containing 22 |
| PLAT | plasminogen activator, tissue type |
| CLDN1 | claudin 1 |
| CPT1A | carnitine palmitoyltransferase 1A |
| BTG2 | BTG anti-proliferation factor 2 |
| MDM2 | MDM2 proto-oncogene |
| CLCA2 | chloride channel accessory 2 |
| SLC7A14 | solute carrier family 7 member 14 |
| INPP5D | inositol polyphosphate-5-phosphatase D |
| DRAM1 | DNA damage regulated autophagy modulator 1 |
| SULF2 | sulfatase 2 |
| FBN1 | fibrillin 1 |
| CRYAB | crystallin alpha B |
| SLC7A11 | solute carrier family 7 member 11 |
| EDA2R | ectodysplasin A2 receptor |
| ACTA2 | actin alpha 2, smooth muscle |
| CCND1 | cyclin D1 |
| TNFRSF10B | TNF receptor superfamily member 10b |
| SPATA18 | spermatogenesis associated 18 |
| CD82 | CD82 molecule |
| ANKRD1 | ankyrin repeat domain 1 |
| HSPA1A | heat shock protein family A (Hsp70) member 1A |
| FAS | Fas cell surface death receptor |
| FASN | fatty acid synthase |
| STC2 | stanniocalcin 2 |
| IGFBP7 | insulin like growth factor binding protein 7 |
| INKA2 | inka box actin regulator 2 |
| HSPA4L | heat shock protein family A (Hsp70) member 4 like |
| DDX60 | DExD/H-box helicase 60 |
| TGFA | transforming growth factor alpha |
| DRAXIN | dorsal inhibitory axon guidance protein |
| WNT5B | Wnt family member 5B |
| DUSP6 | dual specificity phosphatase 6 |
| ETV5 | ETS variant transcription factor 5 |
| SPRY4 | sprouty RTK signaling antagonist 4 |
| EFNB1 | ephrin B1 |
| MARCHF4 | membrane associated ring-CH-type finger 4 |
| PLK3 | polo like kinase 3 |
| PAG1 | phosphoprotein membrane anchor with glycosphingolipid |
| | microdomains 1 |
| HMOX1 | heme oxygenase 1 |
| LACC1 | laccase domain containing 1 |
| PHLDA3 | pleckstrin homology like domain family A member 3 |
| CSMD3 | CUB and Sushi multiple domains 3 |
| ITGA11 | integrin subunit alpha 11 |
| NTN1 | netrin 1 |
| TP5313 | tumor protein p53 inducible protein 3 |
| HLA-DOA | major histocompatibility complex, class II, DO alpha |
| SLCO4A1 | solute carrier organic anion transporter family member 4A1 |
| DHRS2 | dehydrogenase/reductase 2 |
| CA12 | carbonic anhydrase 12 |
| CHAC1 | ChaC glutathione specific gamma-glutamylcyclotransferase 1 |
| HSPE1-MOB4 | HSPE1-MOB4 readthrough |
| DNAH3 | dynein axonemal heavy chain 3 |
| DNAI3 | dynein axonemal intermediate chain 3 |
| RFLNA | refilin A |
| CEACAM1 | CEA cell adhesion molecule 1 |
| ABCB1 | ATP binding cassette subfamily B member 1 |
| NOTCH3 | notch receptor 3 |
| CFH | complement factor H |
| ZDHHC14 | zinc finger DHHC-type palmitoyltransferase 14 |
| ACER2 | alkaline ceramidase 2 |
| SAA1 | serum amyloid A1 |
| FOXO1 | forkhead box O1 |
| NSG1 | neuronal vesicle trafficking associated 1 |
| GREB1 | growth regulating estrogen receptor binding 1 |
| TP53I11 | tumor protein p53 inducible protein 11 |
| PLCL2 | phospholipase C like 2 |
| TMEM59L | transmembrane protein 59 like |
| PPP1R14C | protein phosphatase 1 regulatory inhibitor subunit 14C |
| PTCHD4 | patched domain containing 4 |
| CXCL11 | C-X-C motif chemokine ligand 11 |
| FLRT2 | fibronectin leucine rich transmembrane protein 2 |
| SRGAP3 | SLIT-ROBO Rho GTPase activating protein 3 |
| NPPB | natriuretic peptide B |
| ABCA12 | ATP binding cassette subfamily A member 12 |
| CYGB | cytoglobin |
| AK5 | adenylate kinase 5 |
| CHRM4 | cholinergic receptor muscarinic 4 |
| RHBDL3 | rhomboid like 3 |
| GRHL3 | grainyhead like transcription factor 3 |
| TBX2 | T-box transcription factor 2 |
| GABBR2 | gamma-aminobutyric acid type B receptor subunit 2 |
| NECTIN4 | nectin cell adhesion molecule 4 |
| FOXA1 | forkhead box A1 |
| BLNK | B cell linker |
| MICOS10-NBL1 | MICOS10-NBL1 readthrough |
| IL6 | interleukin 6 |
| POU2F2 | POU class 2 homeobox 2 |
| RTL5 | retrotransposon Gag like 5 |
| GPR87 | G protein-coupled receptor 87 |
| TNFRSF14 | TNF receptor superfamily member 14 |
| UBE2L6 | ubiquitin conjugating enzyme E2 L6 |
| KCNK3 | potassium two pore domain channel subfamily K member 3 |
| CXCL1 | C-X-C motif chemokine ligand 1 |
| NPTXR | neuronal pentraxin receptor |
| ANGPTL4 | angiopoietin like 4 |
| KRTAP2-3 | keratin associated protein 2-3 |
| TRIM55 | tripartite motif containing 55 |
| CXCL8 | C-X-C motif chemokine ligand 8 |
| CDH10 | cadherin 10 |
| ANKRD29 | ankyrin repeat domain 29 |
| ARRDC4 | arrestin domain containing 4 |
| FAM13C | family with sequence similarity 13 member C |
| GATA6 | GATA binding protein 6 |
| FEZ1 | fasciculation and elongation protein zeta 1 |
| LZTS1 | leucine zipper tumor suppressor 1 |
| DOCK4 | dedicator of cytokinesis 4 |
| DNAH12 | dynein axonemal heavy chain 12 |
| SUGCT | succinyl-CoA: glutarate-CoA transferase |
| HERC5 | HECT and RLD domain containing E3 ubiquitin protein ligase 5 |
| IGF2 | insulin like growth factor 2 |
| OAS1 | 2′-5′-oligoadenylate synthetase 1 |
| EMILIN3 | elastin microfibril interfacer 3 |
| MMRN2 | multimerin 2 |
| H2AC11 | H2A clustered histone 11 |
| TLR4 | toll like receptor 4 |
| SCN4B | sodium voltage-gated channel beta subunit 4 |
| ST6GAL1 | ST6 beta-galactoside alpha-2,6-sialyltransferase 1 |
| SGIP1 | SH3GL interacting endocytic adaptor 1 |
| CLTRN | collectrin, amino acid transport regulator |
| SCN2A | sodium voltage-gated channel alpha subunit 2 |
| RTN1 | reticulon 1 |
| LEF1 | lymphoid enhancer binding factor 1 |
| SERPINB7 | serpin family B member 7 |
| SHANK1 | SH3 and multiple ankyrin repeat domains 1 |
| ACSS1 | acyl-CoA synthetase short chain family member 1 |
| HAS3 | hyaluronan synthase 3 |
| PLEKHG1 | pleckstrin homology and RhoGEF domain containing G1 |
| KANK3 | KN motif and ankyrin repeat domains 3 |
| CLEC4E | C-type lectin domain family 4 member E |
| RNASE7 | ribonuclease A family member 7 |
| USP18 | ubiquitin specific peptidase 18 |
| FBXO32 | F-box protein 32 |
| PDE4C | phosphodiesterase 4C |
| HSPA6 | heat shock protein family A (Hsp70) member 6 |
| SPRY1 | sprouty RTK signaling antagonist 1 |
| TMEM179 | transmembrane protein 179 |
| ANK1 | ankyrin 1 |
| SAA2 | serum amyloid A2 |
| H2BC5 | H2B clustered histone 5 |
| FAM43A | family with sequence similarity 43 member A |
| IRGM | immunity related GTPase M |
| GAL3ST4 | galactose-3-O-sulfotransferase 4 |
| NR2F1 | nuclear receptor subfamily 2 group F member 1 |
| CCND2 | cyclin D2 |
| COL13A1 | collagen type XIII alpha 1 chain |
| CCR4 | C-C motif chemokine receptor 4 |
| CNOT6 | CCR4-NOT transcription complex subunit 6 |
| HBA1 | hemoglobin subunit alpha 1 |
| SLC15A3 | solute carrier family 15 member 3 |
| SHC3 | SHC adaptor protein 3 |
| SLC27A2 | solute carrier family 27 member 2 |
| HES1 | hes family bHLH transcription factor 1 |
| GATD3 | glutamine amidotransferase class 1 domain containing 3 |
| NRARP | NOTCH regulated ankyrin repeat protein |
| RTL9 | retrotransposon Gag like 9 |
| POU3F1 | POU class 3 homeobox 1 |
| FAM184A | family with sequence similarity 184 member A |
| REEP1 | receptor accessory protein 1 |
| LRP3 | LDL receptor related protein 3 |
| CASP1 | caspase 1 |
| OPCML | opioid binding protein/cell adhesion molecule like |
| LURAP1L | leucine rich adaptor protein 1 like |
| CD177 | CD177 molecule |
| PNMA2 | PNMA family member 2 |
| ANOS1 | anosmin 1 |
| PADI3 | peptidyl arginine deiminase 3 |
| TMEM158 | transmembrane protein 158 |
| SCN5A | sodium voltage-gated channel alpha subunit 5 |
| SMC1B | structural maintenance of chromosomes 1B |
| LYPD6B | LY6/PLAUR domain containing 6B |
| APOBEC3H | apolipoprotein B mRNA editing enzyme catalytic subunit 3H |
| IFIH1 | interferon induced with helicase C domain 1 |
| KLHL30 | kelch like family member 30 |
| DUSP4 | dual specificity phosphatase 4 |
| LRRC26 | leucine rich repeat containing 26 |
| SNAP25 | synaptosome associated protein 25 |
| ANKRD20A11P | ankyrin repeat domain 20 family member A11, pseudogene |
| SERPING1 | serpin family G member 1 |
| ATP1A3 | ATPase Na+/K+ transporting subunit alpha 3 |
| TMOD1 | tropomodulin 1 |
| WSCD1 | WSC domain containing 1 |
| FBXL16 | F-box and leucine rich repeat protein 16 |
| HTRA1 | HtrA serine peptidase 1 |
| TGFBR3 | transforming growth factor beta receptor 3 |
| EPHX2 | epoxide hydrolase 2 |
| TLR3 | toll like receptor 3 |
| LAMP3 | lysosomal associated membrane protein 3 |
| CYP4F25P | cytochrome P450 family 4 subfamily F member 25, pseudogene |
| COL4A4 | collagen type IV alpha 4 chain |
| MYEOV | myeloma overexpressed |
| STOX2 | storkhead box 2 |
| TG | thyroglobulin |
| ENHO | energy homeostasis associated |
| SLC43A1 | solute carrier family 43 member 1 |
| NRCAM | neuronal cell adhesion molecule |
| CEACAM22P | CEA cell adhesion molecule 22, pseudogene |
| LYNX1 | Ly6/neurotoxin 1 |
| KCNN2 | potassium calcium-activated channel subfamily N member 2 |
| CEMP1 | cementum protein 1 |
| CATSPERD | cation channel sperm associated auxiliary subunit delta |
| CELF2 | CUGBP Elav-like family member 2 |
| HES2 | hes family bHLH transcription factor 2 |
| SMOC1 | SPARC related modular calcium binding 1 |
| BST2 | bone marrow stromal cell antigen 2 |
| NBEAP1 | neurobeachin pseudogene 1 |
| HHATL | hedgehog acyltransferase like |
| CLDN16 | claudin 16 |
| SERPINB2 | serpin family B member 2 |
| PREX1 | phosphatidylinositol-3,4,5-trisphosphate dependent Rac exchange |
| | factor 1 |
| XKR7 | XK related 7 |
| ALOX5 | arachidonate 5-lipoxygenase |
| HMCN2 | hemicentin 2 |
| CARD6 | caspase recruitment domain family member 6 |
| ACVR1C | activin A receptor type 1C |
| CXCL6 | C-X-C motif chemokine ligand 6 |
| C5AR1 | complement C5a receptor 1 |
| ZNF560 | zinc finger protein 560 |
| CYTIP | cytohesin 1 interacting protein |
| TENT5C | terminal nucleotidyltransferase 5C |
| DDO | D-aspartate oxidase |
| DLL4 | delta like canonical Notch ligand 4 |
| OASL | 2′-5′-oligoadenylate synthetase like |
| GRIP2 | glutamate receptor interacting protein 2 |
| RANBP3L | RAN binding protein 3 like |
| C11orf96 | chromosome 11 open reading frame 96 |
| TYRP1 | tyrosinase related protein 1 |
| OR211P | olfactory receptor family 2 subfamily I member 1 pseudogene |
| GRIN1 | glutamate ionotropic receptor NMDA type subunit 1 |
| GPRIN1 | G protein regulated inducer of neurite outgrowth 1 |
| PSG9 | pregnancy specific beta-1-glycoprotein 9 |
| BCAS1 | brain enriched myelin associated protein 1 |
| SLC28A3 | solute carrier family 28 member 3 |
| ADAMTS14 | ADAM metallopeptidase with thrombospondin type 1 motif 14 |
| AKR1B10 | aldo-keto reductase family 1 member B10 |
| FBLN5 | fibulin 5 |
| LHX3 | LIM homeobox 3 |
| MYOCD | myocardin |
| COL25A1 | collagen type XXV alpha 1 chain |
| CSF2 | colony stimulating factor 2 |
| PDK4 | pyruvate dehydrogenase kinase 4 |
| SYN1 | synapsin I |
| KLRG2 | killer cell lectin like receptor G2 |
| PSG3 | pregnancy specific beta-1-glycoprotein 3 |
| ZNF488 | zinc finger protein 488 |
| FBP2 | fructose-bisphosphatase 2 |
| KHSRP | KH-type splicing regulatory protein |
| MUC5AC | mucin 5AC, oligomeric mucus/gel-forming |
| PSG1 | pregnancy specific beta-1-glycoprotein 1 |
| PSG2 | pregnancy specific beta-1-glycoprotein 2 |
| PLD5 | phospholipase D family member 5 |
| ENTREP1 | endosomal transmembrane epsin interactor 1 |
| MAFA | MAF bZIP transcription factor A |
| KLRG1 | killer cell lectin like receptor G1 |
| OFCC1 | orofacial cleft 1 candidate 1 (pseudogene) |
| MT1G | metallothionein 1G |
| FOLR3 | folate receptor gamma |
| PGBD5 | piggyBac transposable element derived 5 |
| IL15RA | interleukin 15 receptor subunit alpha |
| VMO1 | vitelline membrane outer layer 1 homolog |
| LRRC66 | leucine rich repeat containing 66 |
| TFEC | transcription factor EC |
| PAX5 | paired box 5 |
| CEACAM20 | CEA cell adhesion molecule 20 |
| HSD3BP5 | hydroxy-delta-5-steroid dehydrogenase, 3 beta, pseudogene 5 |
| SPECC1L- | SPECC1L-ADORA2A readthrough (NMD candidate) |
| ADORA2A | |
| RGPD1 | RANBP2 like and GRIP domain containing 1 |
| SHC2 | SHC adaptor protein 2 |
| C19orf38 | chromosome 19 open reading frame 38 |
| KRT34 | keratin 34 |
| PPP1R16B | protein phosphatase 1 regulatory subunit 16B |
| TEX19 | testis expressed 19 |
| GTF2IRD2P1 | GTF2I repeat domain containing 2 pseudogene 1 |
| PDE2A | phosphodiesterase 2A |
| ZNF511-PRAP1 | ZNF511-PRAP1 readthrough |
| TINCR | TINCR ubiquitin domain containing |
| RAB39B | RAB39B, member RAS oncogene family |
| EIF3CL | eukaryotic translation initiation factor 3 subunit C like |
| RBP4 | retinol binding protein 4 |
| CCDC168 | coiled-coil domain containing 168 |
| NP1PP1 | nuclear pore complex interacting protein pseudogene 1 |
| H4C14 | H4 clustered histone 14 |
| PCDHB2 | protocadherin beta 2 |
| NTNG2 | netrin G2 |
| OAS2 | 2′-5′-oligoadenylate synthetase 2 |
| GAST | gastrin |
| H4C15 | H4 clustered histone 15 |
| SP140 | SP140 nuclear body protein |
| CORO7-PAM16 | CORO7-PAM16 readthrough |
| MDGA1 | MAM domain containing glycosylphosphatidylinositol anchor 1 |
| SESN1 | sestrin 1 |
| NPDC1 | neural proliferation, differentiation and control 1 |
| CSF1 | colony stimulating factor 1 |
| ZMAT3 | zinc finger matrin-type 3 |
| PTPRU | protein tyrosine phosphatase receptor type U |
| MAST4 | microtubule associated serine/threonine kinase family member 4 |
| FDXR | ferredoxin reductase |
| MAMDC2 | MAM domain containing 2 |
| DUSP5 | dual specificity phosphatase 5 |
| BBC3 | BCL2 binding component 3 |
| ICOSLG | inducible T cell costimulator ligand |
| IFI6 | interferon alpha inducible protein 6 |
| VWCE | von Willebrand factor C and EGF domains |
| WNK4 | WNK lysine deficient protein kinase 4 |
| SLC4A11 | solute carrier family 4 member 11 |
| SLX1B | SLX1 homolog B, structure-specific endonuclease subunit |
| BTBD19 | BTB domain containing 19 |
| BRSK2 | BR serine/threonine kinase 2 |
| EMP1 | epithelial membrane protein 1 |
| CAVIN2 | caveolae associated protein 2 |
| SHC4 | SHC adaptor protein 4 |
| KCNJ12 | potassium inwardly rectifying channel subfamily J member 12 |
| SLC16A12 | solute carrier family 16 member 12 |
| IFI27 | interferon alpha inducible protein 27 |
| F8A2 | coagulation factor VIII associated 2 |
| PDE4A | phosphodiesterase 4A |
| ALDH1A3 | aldehyde dehydrogenase 1 family member A3 |
| STX1A | syntaxin 1A |
| KLLN | killin, p53 regulated DNA replication inhibitor |
| HRNR | hornerin |
| COL17A1 | collagen type XVII alpha 1 chain |
| IL7 | interleukin 7 |
| ASB2 | ankyrin repeat and SOCS box containing 2 |
| SRPX2 | sushi repeat containing protein X-linked 2 |
| KCTD12 | potassium channel tetramerization domain containing 12 |
| NHLH2 | nescient helix-loop-helix 2 |
| ACHE | acetylcholinesterase (Cartwright blood group) |
| SLC52A1 | solute carrier family 52 member 1 |
| GLS2 | glutaminase 2 |
| TBX10 | T-box transcription factor 10 |
| TRPV2 | transient receptor potential cation channel subfamily V member 2 |
| H4C8 | H4 clustered histone 8 |
| ZACN | zinc activated ion channel |
| RPL13AP20 | ribosomal protein L13a pseudogene 20 |
| DYNLT4 | dynein light chain Tctex-type 4 |
| ADCY1 | adenylate cyclase 1 |
| RPL24P8 | RPL24 pseudogene 8 |
| DKK1 | dickkopf WNT signaling pathway inhibitor 1 |
| CALML6 | calmodulin like 6 |
| PRODH | proline dehydrogenase 1 |
| GAD1 | glutamate decarboxylase 1 |
| RNF152 | ring finger protein 152 |
| FGF1 | fibroblast growth factor 1 |
| HEPACAM | hepatic and glial cell adhesion molecule |
| CMPK2 | cytidine/uridine monophosphate kinase 2 |
| FRMPD2 | FERM and PDZ domain containing 2 |
| APOL3 | apolipoprotein L3 |
| KNDC1 | kinase non-catalytic C-lobe domain containing 1 |
| CCDC187 | coiled-coil domain containing 187 |
| FLG2 | filaggrin 2 |
| CHST15 | carbohydrate sulfotransferase 15 |
| RPSAP52 | ribosomal protein SA pseudogene 52 |
| ARC | activity regulated cytoskeleton associated protein |
| NOL3 | nucleolar protein 3 |
| CYP26B1 | cytochrome P450 family 26 subfamily B member 1 |
| NOS1AP | nitric oxide synthase 1 adaptor protein |
| INKA1 | inka box actin regulator 1 |
| KRT23 | keratin 23 |
| RASD2 | RASD family member 2 |
| SMIM10L2A | small integral membrane protein 10 like 2A |
| DUSP13B | dual specificity phosphatase 13B |
| CNTFR | ciliary neurotrophic factor receptor |
| GLDC | glycine decarboxylase |
| KCTD16 | potassium channel tetramerization domain containing 16 |
| TMEM151A | transmembrane protein 151A |
| DMBT1 | deleted in malignant brain tumors 1 |
| KCNQ3 | potassium voltage-gated channel subfamily Q member 3 |
| TTC6 | tetratricopeptide repeat domain 6 |
| PTPRN | protein tyrosine phosphatase receptor type N |
| NPR1 | natriuretic peptide receptor 1 |
| BMPER | BMP binding endothelial regulator |
| EGR2 | early growth response 2 |
| NT5C1B | 5′-nucleotidase, cytosolic IB |
| ALOX15 | arachidonate 15-lipoxygenase |
| DUX4L9 | double homeobox 4 like 9 (pseudogene) |
| IFITM10 | interferon induced transmembrane protein 10 |
| WNT11 | Wnt family member 11 |
| TMEM200A | transmembrane protein 200A |
| ADAM21 | ADAM metallopeptidase domain 21 |
| PSG8 | pregnancy specific beta-1-glycoprotein 8 |
| USH1G | USH1 protein network component sans |
| TP53AIP1 | tumor protein p53 regulated apoptosis inducing protein 1 |
| GBP2 | guanylate binding protein 2 |
| LRRN2 | leucine rich repeat neuronal 2 |
| KLRC1 | killer cell lectin like receptor C1 |
| CASK1N1 | CASK interacting protein 1 |
| MGAT4A | alpha-1,3-mannosyl-glycoprotein 4-beta-N- |
| | acetylglucosaminyltransferase A |
| ANKRD30B | ankyrin repeat domain 30B |
| FGF17 | fibroblast growth factor 17 |
| GPR65 | G protein-coupled receptor 65 |
| IQSEC3 | IQ motif and Sec7 domain ArfGEF 3 |
| DQX1 | DEAQ-box RNA dependent ATPase 1 |
| LKAAEAR1 | LKAAEAR motif containing 1 |
| PSMC1P1 | proteasome 26S subunit, ATPase 1 pseudogene 1 |
| MEP1A | meprin A subunit alpha |
| PALM2 | paralemmin 2 |
| VSTM2L | V-set and transmembrane domain containing 2 like |
| THBD | thrombomodulin |
| CNTNAP3P2 | CNTNAP3 pseudogene 2 |
| HSPB2-C11orf52 | HSPB2-C11orf52 readthrough (NMD candidate) |
| ARHGEF4 | Rho guanine nucleotide exchange factor 4 |
| OSR1 | odd-skipped related transcription factor 1 |
| OSER1 | oxidative stress responsive serine rich 1 |
| OXSR1 | oxidative stress responsive kinase 1 |
| GALNT18 | polypeptide N-acetylgalactosaminyltransferase 18 |
| SCGB1A1 | secretoglobin family 1A member 1 |
| CCIN | calicin |
| AGAP13P | ArfGAP with GTPase domain, ankyrin repeat and PH domain 13, |
| | pseudogene |

TABLE 7

| Gene Symbol | Description |
|---|---|
| SCD | stearoyl-CoA desaturase |
| SLC22A5 | solute carrier family 22 member 5 |
| RARRES2 | retinoic acid receptor responder 2 |
| CDH6 | cadherin 6 |
| IGFBP3 | insulin like growth factor binding protein 3 |
| MMP2 | matrix metallopeptidase 2 |
| CXCL12 | C-X-C motif chemokine ligand 12 |
| HMGCS1 | 3-hydroxy-3-methylglutaryl-CoA synthase 1 |
| CNN1 | calponin 1 |
| CFI | complement factor I |
| PRICKLE1 | prickle planar cell polarity protein 1 |
| INSIG1 | insulin induced gene 1 |
| MN1 | MN1 proto-oncogene, transcriptional regulator |
| LDLR | low density lipoprotein receptor |
| WNT2B | Wnt family member 2B |
| SAMD11 | sterile alpha motif domain containing 11 |
| BMF | Bcl2 modifying factor |
| MTUS1 | microtubule associated scaffold protein 1 |
| PDGFB | platelet derived growth factor subunit B |
| RBM3 | RNA binding motif protein 3 |
| EPHA5 | EPH receptor A5 |
| SCNN1A | sodium channel epithelial 1 subunit alpha |
| ITGA4 | integrin subunit alpha 4 |
| CD24 | CD24 molecule |
| SIPR1 | sphingosine-1-phosphate receptor 1 |
| ERBB3 | erb-b2 receptor tyrosine kinase 3 |
| IGFBP6 | insulin like growth factor binding protein 6 |
| TNFRSF19 | TNF receptor superfamily member 19 |
| LRRC61 | leucine rich repeat containing 61 |
| AHRR | aryl hydrocarbon receptor repressor |
| ZBED10P | zinc finger BED-type containing 10, pseudogene |
| SEMA5A | semaphorin 5A |
| SREBF1 | sterol regulatory element binding transcription factor 1 |
| KRT80 | keratin 80 |
| HAPLN1 | hyaluronan and proteoglycan link protein 1 |
| PDZK1 | PDZ domain containing 1 |
| PTGIS | prostaglandin I2 synthase |
| CRISPLD2 | cysteine rich secretory protein LCCL domain containing 2 |
| HMCN1 | hemicentin 1 |
| SERPINA1 | serpin family A member 1 |
| TCF4 | transcription factor 4 |
| TCF7L2 | transcription factor 7 like 2 |
| COL8A1 | collagen type VIII alpha 1 chain |
| LOXL1 | lysyl oxidase like 1 |
| ADAM19 | ADAM metallopeptidase domain 19 |
| KCNIP1 | potassium voltage-gated channel interacting protein 1 |
| GFRA1 | GDNF family receptor alpha 1 |
| FASN | fatty acid synthase |
| TENM2 | teneurin transmembrane protein 2 |
| COL5A1 | collagen type V alpha 1 chain |
| CARD10 | caspase recruitment domain family member 10 |
| ELF3 | E74 like ETS transcription factor 3 |
| EFNB3 | ephrin B3 |
| CDKN2B | cyclin dependent kinase inhibitor 2B |
| SIM2 | SIM bHLH transcription factor 2 |
| GLIS2 | GLIS family zinc finger 2 |
| CDKN1C | cyclin dependent kinase inhibitor 1C |
| DENND2A | DENN domain containing 2A |
| TRIM6 | tripartite motif containing 6 |
| MUC1 | mucin 1, cell surface associated |
| SHANK2 | SH3 and multiple ankyrin repeat domains 2 |
| ANXA3 | annexin A3 |
| AIF1L | allograft inflammatory factor 1 like |
| GSTM4 | glutathione S-transferase mu 4 |
| LAMA5 | laminin subunit alpha 5 |
| PROS1 | protein S |
| METRNL | meteorin like, glial cell differentiation regulator |
| TUBB2B | tubulin beta 2B class IIb |
| BCAM | basal cell adhesion molecule (Lutheran blood group) |
| BCAT2 | branched chain amino acid transaminase 2 |
| LUM | lumican |
| AP1M2 | adaptor related protein complex 1 subunit mu 2 |
| LFNG | LFNG O-fucosylpeptide 3-beta-N-acetylglucosaminyltransferase |
| RARG | retinoic acid receptor gamma |
| COLEC12 | collectin subfamily member 12 |
| EDN2 | endothelin 2 |
| CAMKK1 | calcium/calmodulin dependent protein kinase kinase 1 |
| LHPP | phospholysine phosphohistidine inorganic pyrophosphate phosphatase |
| ADGRB3 | adhesion G protein-coupled receptor B3 |
| MXRA8 | matrix remodeling associated 8 |
| CCBE1 | collagen and calcium binding EGF domains 1 |
| FHDC1 | FH2 domain containing 1 |
| ARHGAP11B | Rho GTPase activating protein 11B |
| KIF12 | kinesin family member 12 |
| NES | nestin |
| MYL3 | myosin light chain 3 |
| ADAMTS15 | ADAM metallopeptidase with thrombospondin type 1 motif 15 |
| TSPOAP1 | TSPO associated protein 1 |
| SLC1A3 | solute carrier family 1 member 3 |
| PCSK9 | proprotein convertase subtilisin/kexin type 9 |
| SUSD4 | sushi domain containing 4 |
| FJX1 | four-jointed box kinase 1 |
| ANK3 | ankyrin 3 |
| KBTBD11 | kelch repeat and BTB domain containing 11 |
| SGK2 | serum/glucocorticoid regulated kinase 2 |
| SGK3 | serum/glucocorticoid regulated kinase family member 3 |
| C1QL4 | complement C1q like 4 |
| IGFBP5 | insulin like growth factor binding protein 5 |
| THBS3 | thrombospondin 3 |
| COL9A2 | collagen type IX alpha 2 chain |
| GXYLT2 | glucoside xylosyltransferase 2 |
| ATOH8 | atonal bHLH transcription factor 8 |
| DHCR7 | 7-dehydrocholesterol reductase |
| ANO9 | anoctamin 9 |
| SHF | Src homology 2 domain containing F |
| CDH16 | cadherin 16 |
| LHX1 | LIM homeobox 1 |
| RNF43 | ring finger protein 43 |
| ELFN2 | extracellular leucine rich repeat and fibronectin type III domain |
| | containing 2 |
| GDF6 | growth differentiation factor 6 |
| C1orf116 | chromosome 1 open reading frame 116 |
| TRIM58 | tripartite motif containing 58 |
| RUNXITI | RUNX1 partner transcriptional co-repressor 1 |
| CGN | cingulin |
| KIRREL3 | kirre like nephrin family adhesion molecule 3 |
| PIF1 | PIF1 5′-to-3′ DNA helicase |
| ESRP2 | epithelial splicing regulatory protein 2 |
| SLC29A2 | solute carrier family 29 member 2 |
| UNC13D | unc-13 homolog D |
| PLEKHG4B | pleckstrin homology and RhoGEF domain containing G4B |
| FGFR4 | fibroblast growth factor receptor 4 |
| GPR162 | G protein-coupled receptor 162 |
| GPER1 | G protein-coupled estrogen receptor 1 |
| ARHGAP28 | Rho GTPase activating protein 28 |
| ERLEC1P1 | endoplasmic reticulum lectin 1 pseudogene 1 |
| ST6GAL2 | ST6 beta-galactoside alpha-2,6-sialyltransferase 2 |
| PTH1R | parathyroid hormone 1 receptor |
| CHN1 | chimerin 1 |
| RBP1 | retinol binding protein 1 |
| ARID4A | AT-rich interaction domain 4A |
| SGCD | sarcoglycan delta |
| SLITRK5 | SLIT and NTRK like family member 5 |
| TET1 | tet methylcytosine dioxygenase 1 |
| SLC27A3 | solute carrier family 27 member 3 |
| UNC5C | unc-5 netrin receptor C |
| GPRIN2 | G protein regulated inducer of neurite outgrowth 2 |
| ADAMTS6 | ADAM metallopeptidase with thrombospondin type 1 motif 6 |
| GJB4 | gap junction protein beta 4 |
| BMPR1B | bone morphogenetic protein receptor type 1B |
| LRRC7 | leucine rich repeat containing 7 |
| MEX3A | mex-3 RNA binding family member A |
| CD37 | CD37 molecule |
| TRPM2 | transient receptor potential cation channel subfamily M member 2 |
| VWA1 | von Willebrand factor A domain containing 1 |
| SEMA4G | semaphorin 4G |
| CEMIP | cell migration inducing hyaluronidase 1 |
| CDK15 | cyclin dependent kinase 15 |
| GNG2 | G protein subunit gamma 2 |
| FRMD4B | FERM domain containing 4B |
| AGER | advanced glycosylation end-product specific receptor |
| KCNK2 | potassium two pore domain channel subfamily K member 2 |
| TNFRSF13C | TNF receptor superfamily member 13C |
| RND2 | Rho family GTPase 2 |
| OLFML3 | olfactomedin like 3 |
| ESRRG | estrogen related receptor gamma |
| FGF18 | fibroblast growth factor 18 |
| FGFR2 | fibroblast growth factor receptor 2 |
| PAMR1 | peptidase domain containing associated with muscle regeneration 1 |
| SEMA4A | semaphorin 4A |
| CTNND2 | catenin delta 2 |
| AJAP1 | adherens junctions associated protein 1 |
| SLC9A7P1 | solute carrier family 9 member 7 pseudogene 1 |
| FAT2 | FAT atypical cadherin 2 |
| HAVCR2 | hepatitis A virus cellular receptor 2 |
| RARRES1 | retinoic acid receptor responder 1 |
| FBXL13 | F-box and leucine rich repeat protein 13 |
| KCNAB3 | potassium voltage-gated channel subfamily A regulatory beta subunit 3 |
| FOLR1 | folate receptor alpha |
| C1QTNF3 | C1q and TNF related 3 |
| PTP4A3 | protein tyrosine phosphatase 4A3 |
| CGB8 | chorionic gonadotropin subunit beta 8 |
| GPR39 | G protein-coupled receptor 39 |
| BCL2L15 | BCL2 like 15 |
| DIRAS2 | DIRAS family GTPase 2 |
| KL | klotho |
| MSC | musculin |
| MMP19 | matrix metallopeptidase 19 |
| CA11 | carbonic anhydrase 11 |
| GKN1 | gastrokine 1 |
| FOXO6 | forkhead box O6 |
| ECEL1P2 | endothelin converting enzyme like 1 pseudogene 2 |
| COL8A2 | collagen type VIII alpha 2 chain |
| LINC02881 | long intergenic non-protein coding RNA 2881 |
| AMN | amnion associated transmembrane protein |
| ABCD1 | ATP binding cassette subfamily D member 1 |
| HCG22 | HLA complex group 22 (gene/pseudogene) |
| HSD17B8 | hydroxysteroid 17-beta dehydrogenase 8 |
| LRFN5 | leucine rich repeat and fibronectin type III domain containing 5 |
| SORBS2 | sorbin and SH3 domain containing 2 |
| LRRC17 | leucine rich repeat containing 17 |
| TMEM130 | transmembrane protein 130 |
| KLHL14 | kelch like family member 14 |
| NPC1L1 | NPC1 like intracellular cholesterol transporter 1 |
| GPR85 | G protein-coupled receptor 85 |
| PLCH1 | phospholipase C eta 1 |
| SH2D3A | SH2 domain containing 3A |
| CBLN2 | cerebellin 2 precursor |
| ZNF385B | zinc finger protein 385B |
| GAL3ST1 | galactose-3-O-sulfotransferase 1 |
| ITGB6 | integrin subunit beta 6 |
| SLITRK3 | SLIT and NTRK like family member 3 |
| CCT6B | chaperonin containing TCP1 subunit 6B |
| S100A3 | S100 calcium binding protein A3 |
| NOS3 | nitric oxide synthase 3 |
| NANOS3 | nanos C2HC-type zinc finger 3 |
| ST6GALNAC5 | ST6 N-acetylgalactosaminide alpha-2,6-sialyltransferase 5 |
| APOC1 | apolipoprotein C1 |
| ULK4P3 | ULK4 pseudogene 3 |
| HPD | 4-hydroxyphenylpyruvate dioxygenase |
| MEOX1 | mesenchyme homeobox 1 |
| SLC22A3 | solute carrier family 22 member 3 |
| DBP | D-box binding PAR bZIP transcription factor |
| GC | GC vitamin D binding protein |
| HSD17B4 | hydroxysteroid 17-beta dehydrogenase 4 |
| DOCK11 | dedicator of cytokinesis 11 |
| S100A5 | S100 calcium binding protein A5 |
| SLC39A5 | solute carrier family 39 member 5 |
| CLEC18A | C-type lectin domain family 18 member A |
| NBEAP3 | neurobeachin pseudogene 3 |
| SOWAHD | sosondowah ankyrin repeat domain family member D |
| PCDH20 | protocadherin 20 |
| CTNNA3 | catenin alpha 3 |
| KCNMB4 | potassium calcium-activated channel subfamily M regulatory beta |
| | subunit 4 |
| PNMA6A | PNMA family member 6A |
| ISL1 | ISL LIM homeobox 1 |
| TNFRSF6B | TNF receptor superfamily member 6b |
| CBSL | |
| INHBB | inhibin subunit beta B |
| SULF1 | sulfatase 1 |
| TREH | trehalase |
| KRTCAP3 | keratinocyte associated protein 3 |
| CD4 | CD4 molecule |
| CNTN6 | contactin 6 |
| FAM3C2P | family with sequence similarity 3 member C2, pseudogene |
| CERKL | ceramide kinase like |
| PCDH18 | protocadherin 18 |
| NYAP2 | neuronal tyrosine-phosphorylated phosphoinositide-3-kinase adaptor 2 |
| S100A4 | S100 calcium binding protein A4 |
| HPGD | 15-hydroxyprostaglandin dehydrogenase |
| SSC4D | scavenger receptor cysteine rich family member with 4 domains |
| COL24A1 | collagen type XXIV alpha 1 chain |
| CLEC18C | C-type lectin domain family 18 member C |
| CTSW | cathepsin W |
| COL3A1 | collagen type III alpha 1 chain |
| RAMP2 | receptor activity modifying protein 2 |
| GLI1 | GLI family zinc finger 1 |
| ELMOD1 | ELMO domain containing 1 |
| EFHB | EF-hand domain family member B |
| TMEM37 | transmembrane protein 37 |
| TNNC1 | troponin C1, slow skeletal and cardiac type |
| TNNI3 | troponin I3, cardiac type |
| ALDH1A1 | aldehyde dehydrogenase 1 family member A1 |
| NIPSNAP3B | nipsnap homolog 3B |
| MAP1LC3C | microtubule associated protein 1 light chain 3 gamma |
| TMEM155 | |
| TMPRSS5 | transmembrane serine protease 5 |
| TBX1 | T-box transcription factor 1 |
| SOAT2 | sterol O-acyltransferase 2 |
| ENPP5 | ectonucleotide pyrophosphatase/phosphodiesterase family member 5 |
| ZNF467 | zinc finger protein 467 |
| NPY1R | neuropeptide Y receptor Y1 |
| C2orf15 | chromosome 2 open reading frame 15 |
| LAG3 | lymphocyte activating 3 |
| ENAM | enamelin |
| ASIC2 | acid sensing ion channel subunit 2 |
| ADIRF | adipogenesis regulatory factor |
| NPFFR2 | neuropeptide FF receptor 2 |
| EPHA1 | EPH receptor A1 |
| C4B | complement C4B (Chido blood group) |
| C4A | complement C4A (Rodgers blood group) |
| FGB | fibrinogen beta chain |
| GUCY1A1 | guanylate cyclase 1 soluble subunit alpha 1 |
| SLC16A9 | solute carrier family 16 member 9 |
| OTUD7A | OTU deubiquitinase 7A |
| SPDYE9 | speedy/RINGO cell cycle regulator family member E9 |
| CRB2 | crumbs cell polarity complex component 2 |
| SPDYE11 | speedy/RINGO cell cycle regulator family member E11 |
| ID4 | inhibitor of DNA binding 4 |
| ZIC5 | Zic family member 5 |
| TBC1D3C | TBC1 domain family member 3C |
| SLC25A1P5 | solute carrier family 25 member 1 pseudogene 5 |
| TTC39A | tetratricopeptide repeat domain 39A |
| DLEC1 | DLEC1 cilia and flagella associated protein |
| OLFM5P | olfactomedin family member 5, pseudogene |
| SLC22A2 | solute carrier family 22 member 2 |
| SELENOV | selenoprotein V |
| KCNJ4 | potassium inwardly rectifying channel subfamily J member 4 |
| SMIM6 | small integral membrane protein 6 |
| ZBTB45P2 | zinc finger and BTB domain containing 45 pseudogene 2 |
| ZNF90P1 | zinc finger protein 90 pseudogene 1 |
| FAM131C | family with sequence similarity 131 member C |
| ULK4P1 | ULK4 pseudogene 1 |
| ARHGAP8 | Rho GTPase activating protein 8 |
| TIE1 | tyrosine kinase with immunoglobulin like and EGF like domains 1 |
| KRT5 | keratin 5 |
| PRSS8 | serine protease 8 |
| ACKR3 | atypical chemokine receptor 3 |
| RAB7B | RAB7B, member RAS oncogene family |
| THSD7A | thrombospondin type 1 domain containing 7A |
| TTC22 | tetratricopeptide repeat domain 22 |
| PILRA | paired immunoglobin like type 2 receptor alpha |
| RNF180 | ring finger protein 180 |
| GLP2R | glucagon like peptide 2 receptor |
| GPX7 | glutathione peroxidase 7 |
| CLDN7 | claudin 7 |
| SRSF12 | serine and arginine rich splicing factor 12 |
| BTBD18 | BTB domain containing 18 |
| MAP2K6 | mitogen-activated protein kinase kinase 6 |
| ANKRD2 | ankyrin repeat domain 2 |
| LPAR5 | lysophosphatidic acid receptor 5 |
| EPHA6 | EPH receptor A6 |
| ZMYND10 | zinc finger MYND-type containing 10 |
| C1GALT1C1L | C1GALT1 specific chaperone 1 like |
| BAALC | BAALC binder of MAP3K1 and KLF4 |
| TECTA | tectorin alpha |
| ACTG1P10 | actin gamma 1 pseudogene 10 |
| AEBP1 | AE binding protein 1 |
| RENBP | renin binding protein |
| TAMALIN | trafficking regulator and scaffold protein tamalin |
| CCDC85A | coiled-coil domain containing 85A |
| NRSN1 | neurensin 1 |
| SLC5A2 | solute carrier family 5 member 2 |
| C1QTNF5 | C1q and TNF related 5 |
| MFRP | membrane frizzled-related protein |
| KCNE1 | potassium voltage-gated channel subfamily E regulatory subunit 1 |
| TMEM132B | transmembrane protein 132B |
| DLX4 | distal-less homeobox 4 |
| PAQR9 | progestin and adipoQ receptor family member 9 |
| GFRA3 | GDNF family receptor alpha 3 |
| NBPF2P | NBPF member 2, pseudogene |
| PPFIA2 | PTPRF interacting protein alpha 2 |
| GPRIN3 | GPRIN family member 3 |
| FEZF2 | FEZ family zinc finger 2 |
| NCLP1 | nucleolin pseudogene 1 |
| SMIM1 | small integral membrane protein 1 (Vel blood group) |
| OBSCN-AS1 | OBSCN antisense RNA 1 |
| CYP27C1 | cytochrome P450 family 27 subfamily C member 1 |
| RNF175 | ring finger protein 175 |
| GYG2 | glycogenin 2 |
| SPATA32 | spermatogenesis associated 32 |
| LHB | luteinizing hormone subunit beta |
| SLC6A12 | solute carrier family 6 member 12 |
| PCDHGB8P | protocadherin gamma subfamily B, 8 pseudogene |
| ATP8A1 | ATPase phospholipid transporting 8A1 |
| C20orf204 | chromosome 20 open reading frame 204 |
| MFNG | MFNG O-fucosylpeptide 3-beta-N-acetylglucosaminyltransferase |
| MRGPRF | MAS related GPR family member F |
| UNC5CL | unc-5 family C-terminal like |
| CASQ1 | calsequestrin 1 |
| KDF1 | keratinocyte differentiation factor 1 |
| TVP23C- | TVP23C-CDRT4 readthrough |
| CDRT4 | |
| PMFBP1 | polyamine modulated factor 1 binding protein 1 |
| ASPG | asparaginase |
| PDE6B | phosphodiesterase 6B |
| CYP26C1 | cytochrome P450 family 26 subfamily C member 1 |
| FOXP3 | forkhead box P3 |
| ENPP3 | ectonucleotide pyrophosphatase/phosphodiesterase 3 |
| ROBO4 | roundabout guidance receptor 4 |
| IL12B | interleukin 12B |
| ACVRL1 | activin A receptor like type 1 |
| LY75-CD302 | LY75-CD302 readthrough |
| ASS1P12 | argininosuccinate synthetase 1 pseudogene 12 |
| LTB | lymphotoxin beta |
| KLHL31 | kelch like family member 31 |
| IL20RB | interleukin 20 receptor subunit beta |
| LIX1 | limb and CNS expressed 1 |
| SP8 | Sp8 transcription factor |
| POM121L7P | POM121 transmembrane nucleoporin like 7 pseudogene |
| MAL | mal, T cell differentiation protein |
| MRTFA | myocardin related transcription factor A |
| TIRAP | TIR domain containing adaptor protein |
| GRIN3A | glutamate ionotropic receptor NMDA type subunit 3A |
| FREM2 | FRAS1 related extracellular matrix 2 |
| STUM | stum, mechanosensory transduction mediator homolog |
| OMG | oligodendrocyte myelin glycoprotein |
| UPK3B | uroplakin 3B |
| SCGB3A2 | secretoglobin family 3A member 2 |
| PPEF2 | protein phosphatase with EF-hand domain 2 |
| INSYN2B | inhibitory synaptic factor family member 2B |
| CDK3 | cyclin dependent kinase 3 |
| KCNJ5 | potassium inwardly rectifying channel subfamily J member 5 |
| MARK1 | microtubule affinity regulating kinase 1 |
| APELA | apelin receptor early endogenous ligand |
| DAND5 | DAN domain BMP antagonist family member 5 |
| SPEF1 | sperm flagellar 1 |
| RLN2 | relaxin 2 |
| ACTG1P9 | actin gamma 1 pseudogene 9 |
| CD9 | CD9 molecule |
| CRABP2 | cellular retinoic acid binding protein 2 |
| RAB11FIP4 | RAB11 family interacting protein 4 |
| SLCO4C1 | solute carrier organic anion transporter family member 4C1 |
| SMN1 | survival of motor neuron 1, telomeric |
| IL23A | interleukin 23 subunit alpha |
| MPZL2 | myelin protein zero like 2 |
| ITGB1P1 | integrin subunit beta 1 pseudogene 1 |
| SLC4A5 | solute carrier family 4 member 5 |
| SLC4A4 | solute carrier family 4 member 4 |
| WFDC2 | WAP four-disulfide core domain 2 |
| SLC6A16 | solute carrier family 6 member 16 |
| BGN | biglycan |
| ADAMTS9 | ADAM metallopeptidase with thrombospondin type 1 motif 9 |
| RSPH4A | radial spoke head component 4A |
| CNTF | ciliary neurotrophic factor |
| CCDC181 | coiled-coil domain containing 181 |
| ITGB7 | integrin subunit beta 7 |
| ENO4 | enolase 4 |
| OPN1SW | opsin 1, short wave sensitive |
| CDH17 | cadherin 17 |
| TIMD4 | T cell immunoglobulin and mucin domain containing 4 |
| CXXC4 | CXXC finger protein 4 |
| VWF | von Willebrand factor |
| NSUN7 | NOP2/Sun RNA methyltransferase family member 7 |
| IGSF10 | immunoglobulin superfamily member 10 |
| ZNF662 | zinc finger protein 662 |
| FAM110D | family with sequence similarity 110 member D |
| LPAR4 | lysophosphatidic acid receptor 4 |
| DFFBP1 | DNA fragmentation factor subunit beta pseudogene 1 |
| WNT10A | Wnt family member 10A |
| LCK | LCK proto-oncogene, Src family tyrosine kinase |
| PRSS3 | serine protease 3 |
| ZDHHC8BP | ZDHHC8B, pseudogene |
| SGPP2 | sphingosine-1-phosphate phosphatase 2 |
| KIAA0040 | KIAA0040 |
| MTCO1P12 | MT-CO1 pseudogene 12 |
| FAM163A | family with sequence similarity 163 member A |
| SLCO2A1 | solute carrier organic anion transporter family member 2A1 |
| ZFP91-CNTF | ZFP91-CNTF readthrough (NMD candidate) |
| PACRG | parkin coregulated |
| KRT17P7 | keratin 17 pseudogene 7 |
| ZNF736P9Y | zinc finger protein 736 pseudogene 9, Y-linked |
| NPY5R | neuropeptide Y receptor Y5 |
| CEACAM5 | CEA cell adhesion molecule 5 |
| FBXW10B | F-box and WD repeat domain containing 10B |
| ZNF157 | zinc finger protein 157 |
| TTC23L | tetratricopeptide repeat domain 23 like |
| CCDC38 | coiled-coil domain containing 38 |
| RPS3AP6 | RPS3A pseudogene 6 |
| MEI1 | meiotic double-stranded break formation protein 1 |
| PMS2P7 | PMS1 homolog 2, mismatch repair system component pseudogene 7 |
| RPL23AP21 | ribosomal protein L23a pseudogene 21 |
| HENMT1 | HEN methyltransferase 1 |
| PTGDS | prostaglandin D2 synthase |

Referring to FIG. 8A-F, differentially expressed genes (DEGs) with enrichment analysis demonstrates upregulation of genes involved in differentiation and downregulation of genes involved in NF-κB signaling in BT503-treated podocytes. FIG. 8A shows representative heatmap analysis of all 600 DEGs in DMSO-, BT503-treated mice in the setting of LPS versus VEH administration. FIG. 8B shows representative heatmap analysis of WikiPathway and KEGG Pathway for upregulated (red) and downregulated (blue) DEGs (with enrichment for pathways involving genes with KLF15 BS). FIG. 8C and FIG. 8D show representative integrated KEGG Pathway and WikiPathway analysis with ClusterProfiler for all DEGs. FIG. 8E shows representative Gene Set Enrichment Analysis (GSEA) using all DEGs for KEGG NF-κB pathway. FIG. 8F shows representative heatmap analysis of DEGs encompassing NF-κB pathway.

Referring to FIG. 9A-I, BT503 increases KLF15 activity in differentiated human podocytes. FIG. 9A shows representative fold change in KLF15 reporter activity in human podocytes under non-permissive (differentiated) conditions (37° C.) as compared to permissive conditions (33° C.) (relative to DMSO, normalized to Renilla) (n=6-10, ***p<0.001, Kruskal-Wallis test with Dunn's post-test). FIG. 9B shows representative heatmap analysis of all 600 DEGs in DMSO-, BT503-treated mice in 37° C. vs. 33° C. FIG. 9C and FIG. 9D shows representative heatmap analysis of WikiPathway and KEGG Pathway for (FIG. 9C) upregulated and (FIG. 9D) downregulated DEGs (with enrichment for pathways involving genes with KLF15 BS). FIG. 9E shows representative heatmap of top transcription factors from ChIP-enrichment analysis (ChEA) of DEGs. FIG. 9F shows representative heatmap analysis of DEGs encompassing NF-κB pathway. FIG. 9G shows representative Western blot for IKKβ, IKKα, and GAPDH.

### 7. BT503 Directly Inhibits NF-κB Signaling by Targeting IKKβ

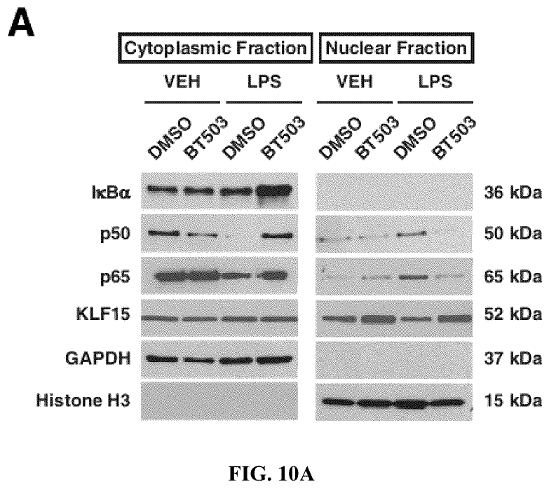

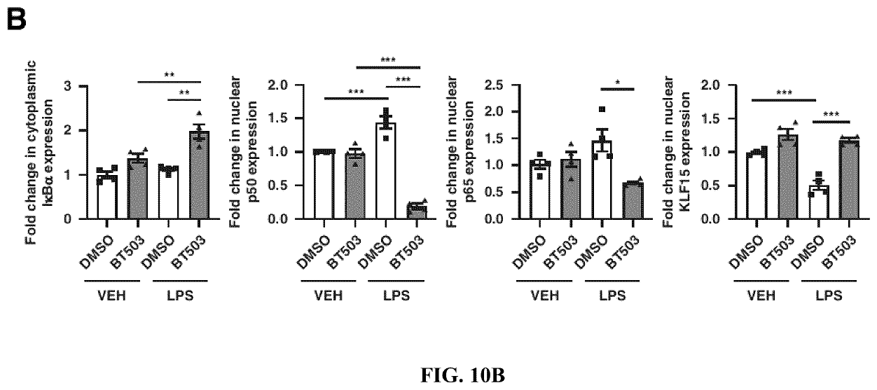

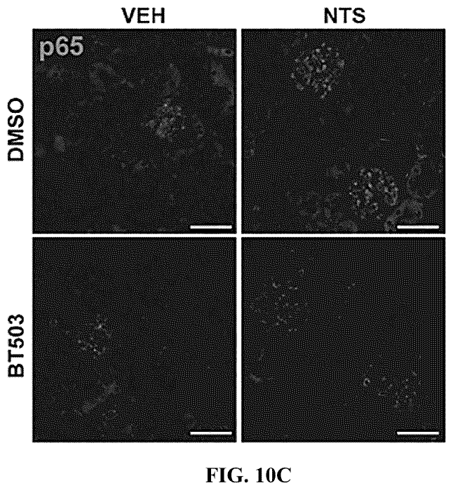

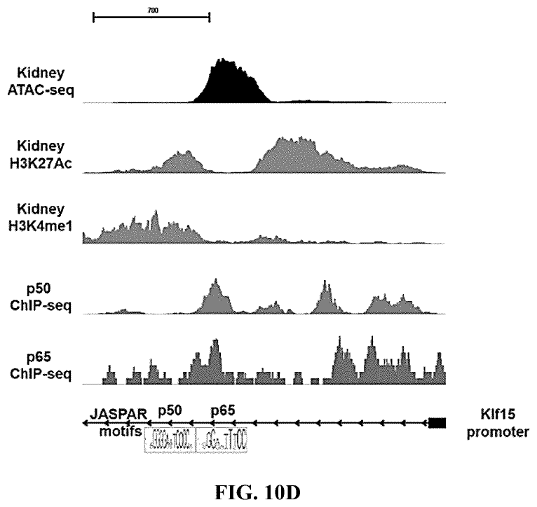

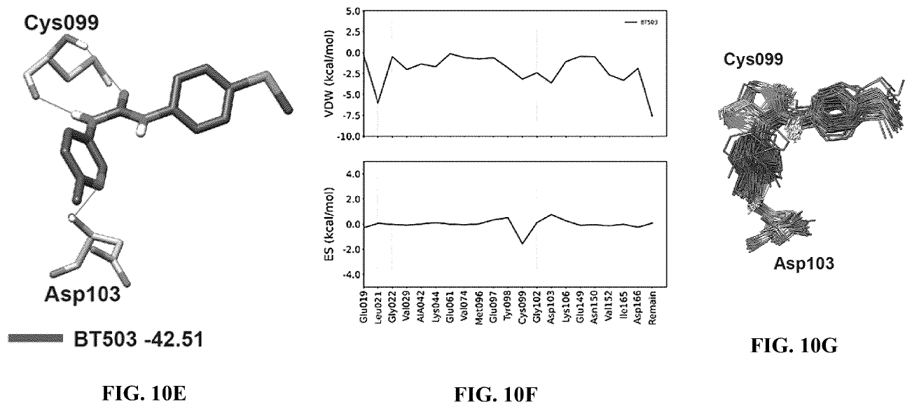

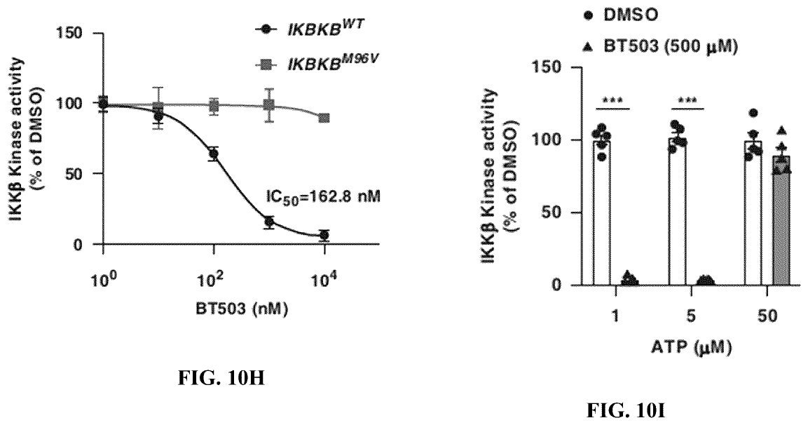

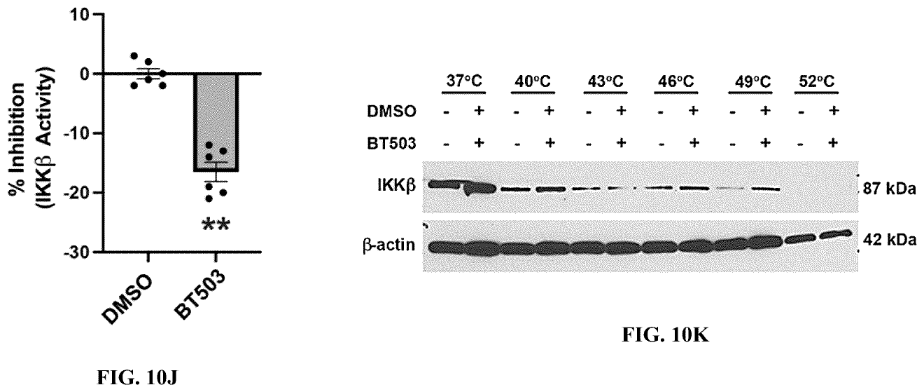

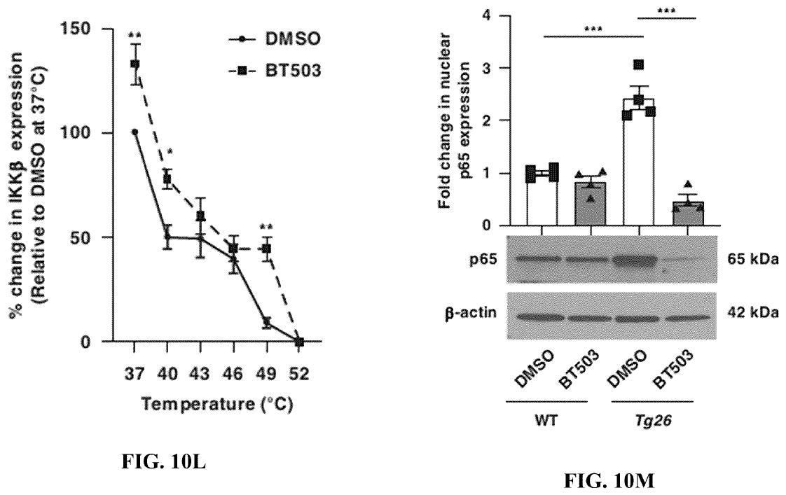

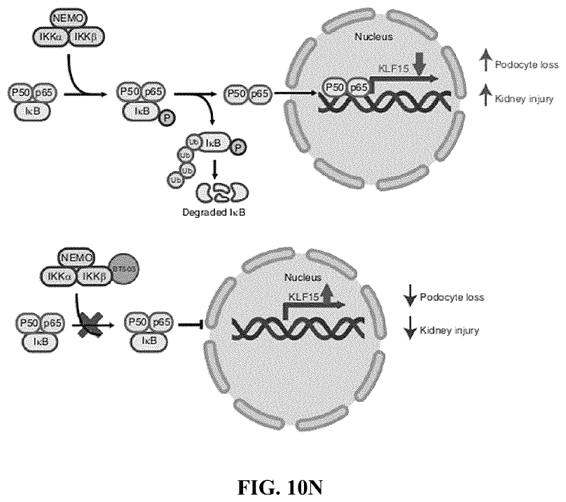

Since the pathways analysis demonstrated an enrichment in pathways involving NF-κB signaling with BT503 treatment, Without wishing to be bound by theory, it was postulated that BT503 targets a component of NF-κB machinery, which might subsequently regulate KLF15. To test this hypothesis, nuclear and cytoplasmic fractions of human podocytes were isolated and treated with BT503 or DMSO in the setting of LPS treatment and conducted western blot of expression of components of canonical NF-κB signaling. While LPS contributed to increased nuclear localization of NF-κB transcription factor dimers (p50 and p65), treatment with BT503 mitigated this translocation (FIG. 10A and FIG. 10B). The NF-κB inhibitory subunit, IκBα, expression was also significantly increased with BT503 as compared to DMSO in the setting of LPS treatment, suggesting BT503 inhibits the phosphorylation of IκBα, thereby preventing its degradation and subsequent translocation of p50 and p65 from the cytosol to nucleus (FIG. 10A and FIG. 10B). It was also confirmed that the NF-κB is activated in the glomeruli post-NTS treatment with induction of p65 expression, which was attenuated with BT503 treatment (FIG. 10C). Since NF-κB signaling has been previously reported to negatively regulate KLF15 (Gao et al. (2011) Kidney Int 79: 987-996; Lu et al. (2013) J Clin Invest 123: 4232-4241; Liu et al. (2018) Mol Med Rep 18: 1987-1994), in silico p50 and p65 motif enrichment was conducted using p50/p65 ChIP-seq data sets (p50 (GSE129618) and p65 (ENCODE ID—ENCSR989LMJ)) to demonstrate that p50/p65 binding sites are located in regions of open chromatin in the putative promoter-proximal enhancer element in the first intron of KLF15 (FIG. 10D), suggesting that the activation of NF-κB signaling directly suppresses KLF15 expression.

Iκκ complex, composed of IKKα, IKKβ, and NEMO/IKKγ, is the central regulator of NF-κB signaling by phosphorylating IκBα, with canonical signaling mediated by IKKβ and noncanonical signaling via IKKα (A. Israel, T(2010) Cold Spring Harb Perspect Biol 2, a000158). Since BT503 induced KLF15 activity under nonpermissive conditions as compared to permissive conditions (FIG. 9A), interrogation of DEGs from RNA-sequencing of BT503 human podocytes in this setting demonstrated that the key component of Iκκ complex, IKBKB was significantly upregulated in nonpermissive conditions (i.e., differentiated human podocytes) as compared to permissive conditions (FIG. 9F). Furthermore, IKKβ was highly enriched under these nonpermissive conditions as compared to permissive conditions, suggesting the kinase might serve as a target for BT503, leading to an inhibition in canonical NF-κB signaling with a subsequent induction of KLF15 (FIG. 9G and FIG. 9H). Interestingly, IKKα levels were similar under both conditions (FIG. 9G and FIG. 9I).

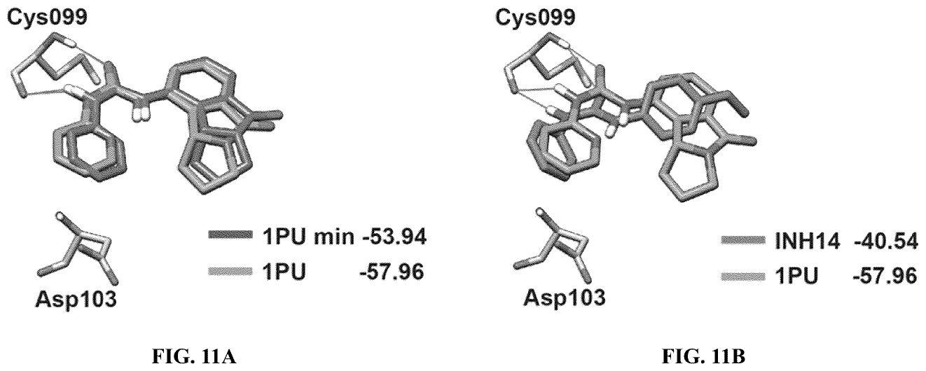

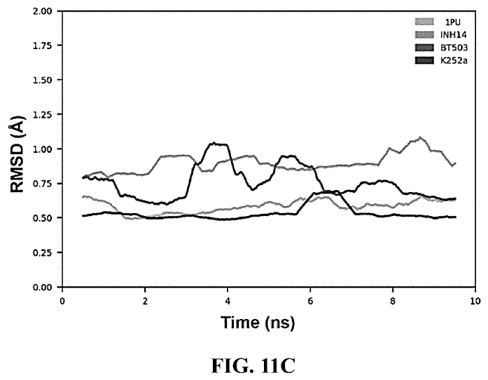

To identify and quantify the most likely binding pose for BT503 with IKKβ, molecular modeling was utilized by combining previously reported modeling on a related small molecule inhibitor of IKKβ, INH14 (M. Drexel, J. Kirchmair, S. Santos-Sierra, (2019) Chembiochem 20, 710-717), with a structurally related inhibitor, called 1PU, that has been co-crystallized with a CDK4 mimic of CDK2 (M. Ikuta et al., (2001) J Biol Chem 276, 27548-27554), which shares homology with IKKβ. This initial quantitative analysis demonstrates binding interactions for BT503 with IKKβ through energy minimization, docking, molecular footprints, and molecular dynamics (FIG. 10E). Additionally, this binding pose was also validated for previously reported ligands, INH14 and 1PU, with IKKβ (FIG. 11A and FIG. 11B). Docking BT503 yields a nearly identical 3D pose to previously reported ligand, INH14, with similar energy scores (−42.51 vs −40.54 kcal/mol) (FIG. 10E and FIG. 11B). The predicted BT503 pose maintains the favorable electrostatic (ES) interactions between the urea and Cys099, and the rotated pyridine ring places a nitrogen H-bond acceptor within range of Asp103. Taken together, these docking studies provide strong support for the ligand geometries in FIG. 10E and provide confidence that the current DOCK protocols are suitable for modeling IKKβ.

To investigate which residues are most likely to contribute to ligand binding in IKKβ, the DOCK scores for BT503 were decomposed into their respective per-residue Van der Waals (VDW) and ES components (termed footprints) (FIG. 10F). Here, the 20 highest contributing residues are explicitly shown and the sum of all other interactions are grouped together into the residue labeled “Remain.” Importantly, the interaction energy profiles are qualitatively similar across all four ligands, which corresponds to the structural overlap observed in FIG. 10E and FIG. 11B.

For computed electrostatic contributions, BT503 is observed to make favorable interactions of ca. −2 kcal/mol at position Cys099, which corresponds to the two H-bonds with the protein backbone shown in FIG. 10F. Despite the proximity of a potential H-bond acceptor within range of Asp103, BT503 shows a roughly 0.3 kcal/mol ES repulsive at this position (FIG. 10F). While the interaction with the backbone is expected to be favorable, the larger overall unfavorable ES interaction with this specific residue could be attributed to repulsion between the BT503 pyridine nitrogen and the Asp103 side chain. Overall, these footprints help to quantify how BT503 is predicted to lock into a specific binding geometry with IKKβ. They also support that binding is driven by interactions between the trans-diamide and the Cys099 backbone, analogous to those observed in the x-ray structure of 1PU in the CDK4 mimic at position Leu083 (pdb code 1GIH)(M. Ikuta et al., (2001) J Biol Chem 276, 27548-27554).

To further assess the validity of the predictions, molecular dynamics (MD) simulations were performed for each protein-ligand complex. FIG. 11C plots ligand RMSD as a function of time from solvated IKKβ complexes with INH14 (orange), and BT503 (green). As a control, the cognate ligand K252a (black) was also simulated to validate the robustness of the MD protocol. Importantly, all of the ligand poses were stable in the IKKβ binding site as judged by plateaued box-averaged RMSD plots versus time (N=100 frames, 10 ns) with only minor deviation from their docked (INH14, BT503) or x-ray (K252a) pose. The small deviations for the K252a control (ca. 0.5 Å RMSD, black) provided evidence the simulation protocols and force field parameters are robust.

An ensemble overlay of structures taken from the BT503 trajectory (every 20th MD frame) was provided to visually assess ligand and sidechain dynamics and the stability of H-bonding (FIG. 10G). Here, ligand and sidechains remain tightly locked in place and the consistent cis-trans urea orientation of BT503 remains pointed at Cys099 which indicates that the two backbone H-bonds observed from docking are maintained during MD (magenta lines). In many of the snapshots, the BT503 pyridine ring nitrogen is oriented towards Asp103 which is also within H-bonding distance of the protein backbone. The BT503 sulfide is consistently oriented in the general direction of the Asp166 backbone amide (residue not shown) which would also facilitate H-bonding.

To assess how the binding interaction profiles might vary, time averaged footprints for 1PU and BT503 were computed (FIG. 11D). Overall, the averaged footprints are very similar to those from the single point docking calculations for BT503 (FIG. 10F) and the small error bars indicate good energetic stability over time. Notably for BT503, the slight unfavorable ES interactions originally observed in the docking profiles at Asp103 (and to a lesser extent Tyr098) were relaxed during MD, and VDW packing with Met096 and Tyr098 was improved. Interestingly, the net result is a much tighter overlap between the 1PU and BT503 footprint profiles after MD-based sampling.

To validate this interaction between BT503 and IKKβ experimentally, IKKβ kinase activity was initially measured in the setting of DMSO and BT503 treatment to demonstrate a significant reduction in IKKβ activity with increasing concentration of BT503 (IC₅₀=163 nM) as compared to DMSO (FIG. 10H). In addition, this BT503-mediated inhibition of kinase activity was mitigated at higher ATP concentrations (50 μM), suggesting ATP-dependency (FIG. 10I). To test the specificity of BT503 to IKKβ inhibition, a IKBKB gatekeeper mutant was generated by mutating methionine to valine (IKBKB^{M96V}) in the ATP docking site of IKKβ. While the IKBKB^{M96V} mutant has similar kinase activity as compared to IKBKB^{WT} at baseline, treatment with BT503 demonstrated a resistance to IKKβ kinase inhibition in the IKBKB^{M96V} mutant as compared to IKBKB^{WT} (FIG. 10H), thereby suggesting a direct causal link between IKKβ inhibition and the administration of BT503. Thermal shift assay in human podocytes treated with BT503 or DMSO showed that IKKβ stability was reduced in DMSO as compared to BT503 treatment, further validating the physical BT503-IKKβ interaction (FIG. 10J-L). Finally, p65 expression was reduced in BT503-treated Tg26 mice, confirming that BT503 inhibits NF-κB signaling in vivo (FIG. 10M). Collectively, these data suggest that BT503 directly inhibited IKKβ from phosphorylating IκBα, NF-κB inhibitory subunit, which prevented the nuclear translocation of NF-κB dimers, and, in turn, restored KLF15 levels under podocyte stress (FIG. 10N).

Referring to FIG. 10A-N, BT503 inhibits IKKβ activity, leading to the inhibition of p50/p65 translocation and restoration of KLF15 in the setting of cell stress. FIG. 10A and FIG. 10B show representative Western blot with quantification of densitometry for p50, p65, IκBα, GAPDH, and Histone H3 from nuclear and cytoplasmic fractions in DMSO-vs. BT503-treated podocytes in the setting of LPS and VEH treatment. FIG. 10C shows representative immunostaining for p65 in DMSO- vs. BT503-treated podocytes in the setting of NTS and VEH treatment. FIG. 10D shows representative mapping of open chromatin from kidney ATAC-seq, H3K27Ac, and H3K4me1 ChIP-seq data from the ENCODE consortium to show putative promoter-proximal enhancer element in the first intron of KLF15. Mapping of aforementioned regulatory element to show overlap of ChIP-seq-determined binding sites for p50 (GSE129618) and p65 (ENCODE ID—ENCSR989LMJ) (canonical motifs are show for each by the JASPAR motif position weight matrices (PWMs) listed along the ENSEMBL gene track). FIG. 10E shows docked BT503 pose on IKKβ. Only two protein residues shown for clarity. Potential H-bonds in magenta. DOCK scores reported in kcal/mol. FIG. 10F shows representative-data illustrating molecular footprints (per residue energy breakdown) for docked BT503 (green) with IKKβ. The Van der Waals (VDW) (top) and electrostatic (ES) (bottom) residue lists correspond to the top 20 most favorable residues. Energies reported in kcal/mol. FIG. 10G shows a representative ensemble overlay (N=100 frames) for BT503 (green) showing key IKKβ residues (gray) involved in hydrogen bonding (magenta). FIG. 10H shows representative data illustrating the IKKβ kinase activity for BT503 and DMSO in IKBKB^{WT} and IKBKB^{M96V}(% relative to DMSO). IC₅₀ for BT503 in IKKB^{WT} is shown (***P<0.001, two-way ANOVA with Bonferroni post-test). FIG. 10I shows representative data illustrating the IKKβ kinase activity for BT503 and DMSO at ATP concentrations of 1, 5, and 50 μM (% relative to DMSO) (n=5, ***P<0.001, multiple Mann-Whitney tests). FIG. 10J shows representative % inhibition of IKKβ kinase activity (**p<0.01, Mann-Whitney Test). FIG. 10K and FIG. 10L show representative Western blot with quantification of densitometry for IKKβ and j-actin from the cellular thermal shift assay in human podocytes treated with DMSO or BT503 (1 μM). Representative images of three independent experiments are shown (n=4, *P<0.05, **P<0.01, two-way ANOVA with Bonferroni post-test). FIG. 10M shows representative Western blot data with quantification of densitometry for p65 and β-actin from kidney lysates in DMSO-treated versus BT503-treated Tg26 mice. Representative blots from four different experiments are shown (n-4, ***P<0.001 compared with all other groups, Kruskal-Wallis test with Dunn post-test). FIG. 10N shows a representative schematic illustrating (top panel) that activated NF-κB signaling suppresses KLF15 expression and subsequent podocyte loss and kidney injury as compared with (bottom panel) BT503-mediated inhibition of IKKβ, which inactivates NF-κB signaling and subsequently restors KLF15 expression, leading to a reduction in podocyte loss and kidney injury. ATAC-seq, assay for transposase-accessible chromatin with sequencing; ChIP-seq, chromatin immunoprecipitation sequencing; ENCODE, encyclopedia of DNA elements; ES, electrostatic; IKKβ, inhibitor of nuclear factor kappa-B kinase subunit beta; NF-kappa-B essential modifier; PWM, position weight matrix; VDW, Van der Waals.

Referring to FIG. 11A-D, molecular footprints for energy-minimized 1PU, docked 1PU, and INH14 on IKKβ are shown. FIG. 11A and FIG. 11B show representative molecular footprints (per residue energy breakdown) for energy-minimized 1PU (purple), docked 1PU (light blue), and INH14 (orange) on IKKβ. Only three protein residues shown for clarity. Potential H-bonds (magenta). DOCK scores reported in kcal/mol. FIG. 11C shows representative ligand root-mean square deviations (RMSD) from simulations in IKKβ relative to their original docked (1PU, INH14, BT503) or cognate ligand pose (K252a x-ray pose) as a function of simulation time (box car averaged over 200 frames, 2000 frames total). FIG. 11D shows time averaged molecular footprints (N=100 frames, 10 ns) for 1PU (cyan) and BT503 (green) with error bars representing standard deviations. The VDW (top) and ES (bottom) residue lists correspond to the top 20 most favorable residues (plus remainder residues labeled: Remain). Energies in kcal/mol.

### 8. BT503 Demonstrates Low Cytotoxicity

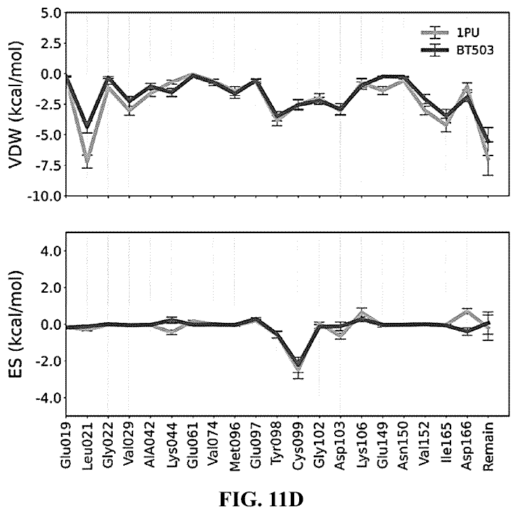

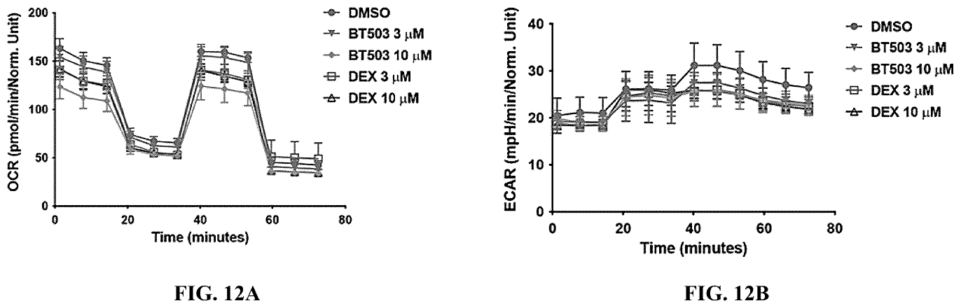

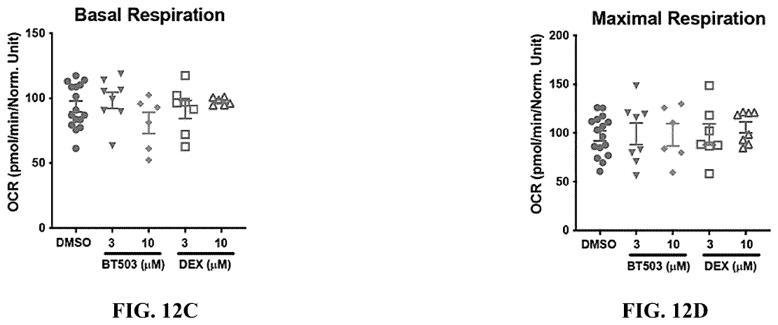

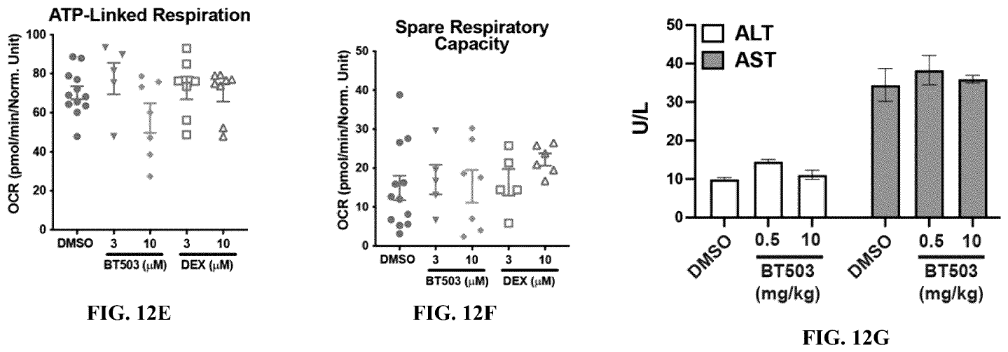

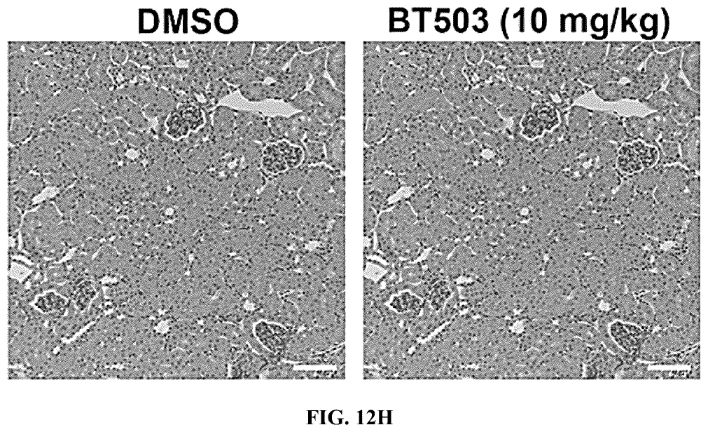

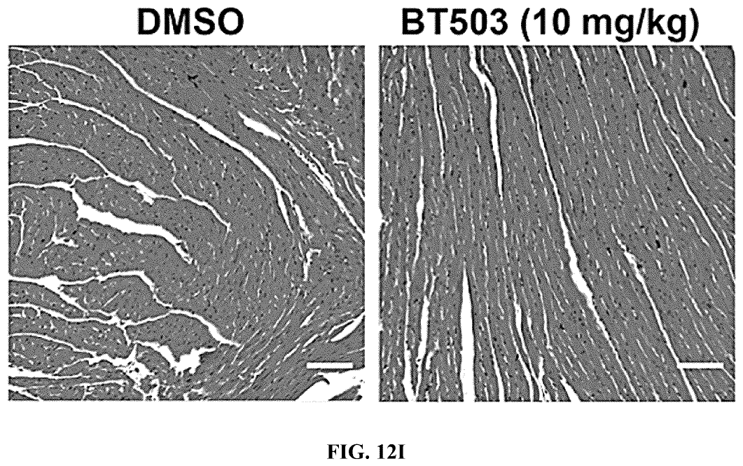

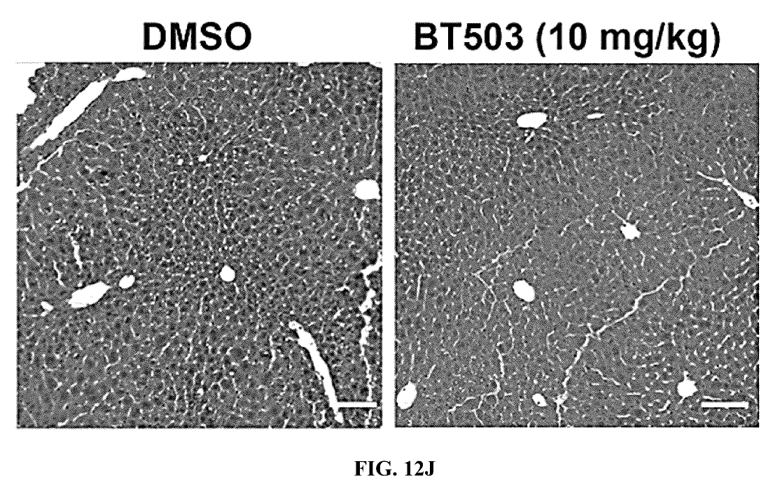

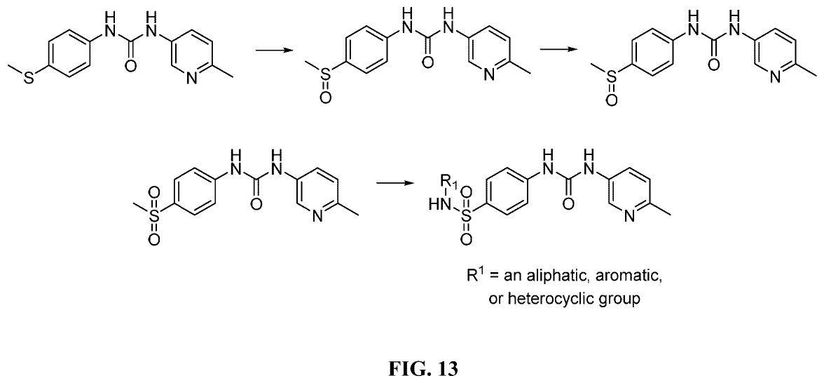

To initially test the cellular toxicity of BT503, BT503 was screened in hERG Human Potassium Ion Channel Cell Based QPatch CiPA Assay using CHO-K1 cells. BT503 demonstrated low inhibition as compared to positive control at low to high concentration ranges (3-10 μM) (Table 8). Furthermore, differentiated human podocytes treated with BT503 showed no significant differences in oxygen consumption rate (OCR) and extracellular acidification rate (ECAR) as compared to DMSO or DEX (FIG. 12A and FIG. 12B). No significant differences were also observed in basal respiration, maximal respiration, ATP-linked respiration, and spare respiratory capacity at both concentrations of BT503 (3-10 μM) (FIG. 12C-F). Furthermore, BT503 showed no significant difference in aspartate aminotransferase (AST), alanine transaminase (ALT) levels and histological changes in the kidney, heart, and liver tissue as compared to DMSO treatment (FIG. 12G-J). Without wishing to be bound by theory, these data suggest that BT503 has no significant cytotoxicity at therapeutic and supratherapeutic ranges.

TABLE 8

| | Concentration | % inhibition | | |
|---|---|---|---|---|
| Compound | (μM) | n1 | n2 | mean |
| BT503 | 3 | −2.01 | 4.41 | 1.20 |
| | 10 | −0.08 | 12.57 | 6.25 |
| 0.33% DMSO | | −5.42 | −6.70 | −6.06 |
| (time-matched | | −7.26 | −7.95 | −7.60 |
| vehicle control) | | −8.92 | −5.90 | −7.41 |
| E-4031 (positive | 0.001 | 6.30 | 7.68 | 6.99 |
| reference | 0.003 | 11.83 | 10.89 | 11.36 |
| control) | 0.01 | 19.86 | 18.72 | 19.29 |
| | 0.03 | 24.25 | 9.95 | 17.10 |
| | 0.1 | 85.08 | 74.34 | 79.71 |
| | 0.3 | 97.88 | 96.66 | 97.27 |

Referring to FIG. 12A-J, molecular footprints for energy-minimized 1PU, docked 1PU, and INH14 on IKKβ are shown. FIG. 12A shows representative oxygen consumption rate (OCR) and FIG. 12B shows representative extracellular acidification rate (ECAR) with quantification of FIG. 12C basal OCR, FIG. 12D maximal respiration, FIG. 12E ATP-linked respiration, and FIG. 12F spare respiratory capacity for podocytes treated with DMSO, BT503 (3 μM, 10 μM), and DEX (3 μM, 10 μM) (n=8 per group). FIG. 12G shows representative ALT and AST levels in DMSO, BT503-treated mice (0.5, 10 mg/kg) (n=3 per group). Periodic-acid schiff staining of kidney cortex FIG. 12H, heart FIG. 12I, and liver tissues FIG. 12J in DMSO-, BT503-treated (10 mg/kg) mice. Representative images of three independent experiments are shown.

### 9. Discussion

Podocyte injury and loss is critical to the development and progression of glomerulosclerosis and eventual development of CKD. Based on previously reported studies, the salutary effects of KLF15 in podocytes was leveraged (S. K. Mallipattu, J. C. He, (2016) Am J Physiol Renal Physiol 311, F46-51, S. K. Mallipattu et al., (2017) J Am Soc Nephrol 28, 166-184, S. K. Mallipattu et al., (2012). J Biol Chem 287, 19122-19135) to initially develop and validate a novel HTS human podocyte screening assay to identify KLF15 agonists. The top “hit” from the initial screen, C-7, underwent SAR study to synthesize novel structural analogues with a goal of improving therapeutic efficacy and druggability. Of these novel structural analogues, BT503 induced KLF15 activity while demonstrating a salutary role in differentiated human podocytes and in three independent proteinuric murine models without significant cytotoxicity. In addition, the use of BT503 in cultured human podocytes and in mice reduced the DEX dose required for its therapeutic effects. A combination of RNA-sequencing, molecular modeling and experimental validation demonstrates that BT503 inhibits canonical NF-κB signaling by directly targeting IKKβ from phosphorylating the IκBα, NF-κB inhibitory subunit, which prevents the NF-κB dimers from suppressing KLF15 in the setting of podocyte stress.

BT503 has a urea linker, which is commonly found in many clinically used bioactive compounds (R. Ronchetti, et al., (2021) Rsc Med Chem 12, 1046-1064, S. Ghosh, et al., (2022) J Indian Chem Soc 99). Urea moiety has served as a backbone of many known kinase inhibitors (R. Listro et al., (2022) Front Chem 10). A key advantage of urea-based compounds as a therapy for kidney disease is their ability to form hydrogen bond interactions due to the presence of two hydrogen bond donors and one acceptor which affects their solubility as well as interactions with target proteins. In addition, the urea linker makes the compounds conformationally restricted (S. Ghosh, et al., (2022) J Indian Chem Soc 99), which helps improve their specificity and potency (P. D. M. Pinheiro, et al., (2019) Curr Top Med Chem 19, 1712-1733). However, there can be limitations, in some instances having the urea linker can reduce the solubility and permeability of the compound (S. Kumari, et al., (2020) Journal of Medicinal Chemistry 63, 12290-12358). Without wishing to be bound by theory, the direct target of BT503 is IKKβ, additional SAR studies could be utilized to generate analogues of BT503, which maintain the predicted H-bonding with Cys099, and enhance efficacy without compromising druggability and toxicity (i.e., modifications that enhance interactions with nearby residues Lys044, Tyr098, Asp103). Future pharmacodynamic and pharmacokinetic studies will also be necessary to optimize BT503 prior to clinical use.

Three independent proteinuric murine models were utilized to test the therapeutic role of BT503. While the administration of BT503 attenuated albuminuria and podocyte effacement post LPS-treatment, it is a transient model of podocyte injury without podocyte loss or glomerulosclerosis (S. K. Mallipattu, J. C. He, (2016) Am J Physiol Renal Physiol 311, F46-51, S. K. Mallipattu et al., (2012). J Biol Chem 287, 19122-19135, J. Reiser et al., (2004) J Clin Invest 113, 1390-1397, H. W. Lee et al., (2015) J Am Soc Nephrol 26, 2741-2752). Therefore, the use of NTS and the Tg26 models were used to demonstrate that BT503 not only abrogated albuminuria, but preserved podocyte number and attenuated eventual glomerulosclerosis. Since the Tg26 mice develops albuminuria starting at 4 weeks of age with progressive podocyte loss, glomerulosclerosis, reduced kidney function, and eventual kidney fibrosis (S. K. Mallipattu et al., (2017) J Am Soc Nephrol 28, 166-184, P. Dickie et al., (1991) Virology 185, 109-119), treatment with BT503 at 8 weeks (i.e., subsequent to development of podocyte injury and glomerulosclerosis), significantly reduced the % of FSGS lesions while preserving podocyte number and kidney function. Therefore, the studies suggest the potential role of BT503 in preventing as well as ameliorating podocyte injury and glomerulosclerosis. Additionally, in vivo PK studies exploring the optimal dose, frequency, and duration of BT503 treatment in these specific proteinuric models might further substantiate the potential therapeutic role of BT503 in podocytopathies.

There are several studies demonstrating the detrimental effects of NF-κB activation in podocyte injury and glomerular disease (E. A. Korte et al., (2017) Am J Pathol 187, 2799-2810, N. Prakoura et al., (2017) J Am Soc Nephrol 28, 1475-1490, N. Song, et al., (2019) Front Immunol 10, 815, S. Brahler et al., I(2012) American Journal of Physiology-Renal Physiology 303, F1473-F1485, H. Bao, et al., (2015) Kidney Int 87, 1176-1190). In addition, activation of NF-κB signaling as well as single nucleotide polymorphisms in components of NF-κB signaling have been reported in human glomerular diseases (D. J. Caster et al., (2013) J Am Soc Nephrol 24, 1743-1754, J. Y. Zhang et al., (2018) Proc Natl Acad Sci USA 115, 3446-3451). However, there are limited studies exploring the salutary effects of inhibiting NF-κB in glomerular disease. Studies that show the use of small molecule inhibitors of NF-κB signaling in kidney disease have not clearly demonstrated whether this is attributable to a direct mechanism of action or a result of off-target effects. Furthermore, the use of current therapeutic strategies for podocytopathies, such as renin-angiotensin-aldosterone system (RAAS) blockade and GCs, have been shown to inhibit canonical NF-κB signaling (J. I. Lee, G. J. Burckart, (1998) J Clin Pharmacol 38, 981-993, L. I. McKay, J. A. Cidlowski, (1999) Endocr Rev 20, 435-459, F. T. H. Lee et al., (2004) Journal of the American Society of Nephrology 15, 2139-2151, R. Patel, et al., (2014) Mol Endocrinol 28, 999-1011), highlighting the potential significance of direct NF-κB inhibition in human glomerular diseases. It was shown that the combination of BT503 and DEX reduced the DEX dose needed to attenuate albuminuria. In addition, mutating the GRE on KLF15 did not mitigate the effects of BT503, suggesting the effects of BT503 are, in part, independent of GR signaling in podocytes. Since the use of chronic and high-dose GCs is riddled with systemic toxicities, identification of small molecules that could reduce the dose of GCs necessary to maintain therapeutic efficacy would be a major advance in mitigating the adverse effects associated with these agents. In addition, future studies investigating whether the concurrent use of BT503 with RAAS blockade is synergistic in preventing or restoring podocyte loss and glomerular injury is clinically relevant. The knockdown of KLF15 abrogated the salutary effects of BT503 in cultured podocytes was reported, suggesting that the effects BT503-IKKβ inhibition is mediated through KLF15. Without wishing to be bound by theory, this novel human podocyte-based KLF15 HTS could be used to screen additional small molecules that target modulators of KLF15. Finally, the use of BT503 might have a potential therapeutic benefit in other conditions where aberrant activation of canonical NF-κB signaling contributes to disease development and/or progression.

### F. References

1. Chronic Kidney Disease in the United States, 2023. Atlanta, GA: US Department of Health and Human Services, Centers for Disease Control and Prevention; 2023. (2023).
2. A. Meyrier, Mechanisms of disease: focal segmental glomerulosclerosis. Nature clinical practice. Nephrology 1, 44-54 (2005).
3. L. Barisoni, Podocyte biology in segmental sclerosis and progressive glomerular injury. Adv Chronic Kidney Dis 19, 76-83 (2012).
4. M. van Husen, M. J. Kemper, New therapies in steroid-sensitive and steroid-resistant idiopathic nephrotic syndrome. Pediatr Nephrol 26, 881-892 (2011).
5. C. Ponticelli, F. Locatelli, Glucocorticoids in the Treatment of Glomerular Diseases: Pitfalls and Pearls. Clin J Am Soc Nephrol 13, 815-822 (2018).
6. P. J. Barnes, Anti-inflammatory actions of glucocorticoids: molecular mechanisms. Clin Sci (Lond) 94, 557-572 (1998).
7. C. Riccardi, S. Bruscoli, G. Migliorati, Molecular mechanisms of immunomodulatory activity of glucocorticoids. Pharmacol Res 45, 361-368 (2002).
8. E. Schonenberger, J. H. Ehrich, H. Haller, M. Schiffer, The podocyte as a direct target of immunosuppressive agents. Nephrol Dial Transplant 26, 18-24 (2011).
9. A. Guess et al., Dose- and time-dependent glucocorticoid receptor signaling in podocytes. Am J Physiol Renal Physiol 299, F845-853 (2010).
10. T. Wada, J. W. Pippin, M. Nangaku, S. J. Shankland, Dexamethasone's prosurvival benefits in podocytes require extracellular signal-regulated kinase phosphorylation. Nephron Exp Nephrol 109, e8-19 (2008).
11. T. Wada, J. W. Pippin, C. B. Marshall, S. V. Griffin, S. J. Shankland, Dexamethasone prevents podocyte apoptosis induced by puromycin aminonucleoside: role of p53 and Bcl-2-related family proteins. J Am Soc Nephrol 16, 2615-2625 (2005).
12. C. Y. Xing et al., Direct effects of dexamethasone on human podocytes. Kidney Int 70, 1038-1045 (2006).
13. H. Zhou et al., Loss of the podocyte glucocorticoid receptor exacerbates proteinuria after injury. Sci Rep 7, 9833 (2017).
14. S. K. Mallipattu, J. C. He, The podocyte as a direct target for treatment of glomerular disease? Am J Physiol Renal Physiol 311, F46-51 (2016).
15. E. Torban et al., From podocyte biology to novel cures for glomerular disease. Kidney Int 96, 850-861 (2019).
16. A. S. De Vriese, J. F. Wetzels, R. J. Glassock, S. Sethi, F. C. Fervenza, Therapeutic trials in adult FSGS: lessons learned and the road forward. Nat Rev Nephrol 17, 619-630 (2021).
17. S. K. Mallipattu et al., Kruppel-Like Factor 15 Mediates Glucocorticoid-Induced Restoration of Podocyte Differentiation Markers. J Am Soc Nephrol 28, 166-184 (2017).
18. S. K. Mallipattu, C. C. Estrada, J. C. He, The critical role of Kruppel-like factors in kidney disease. Am J Physiol Renal Physiol 312, F259-F265 (2017).
19. X. Gu et al., The loss of Kruppel-like factor 15 in Foxd1+ stromal cells exacerbates kidney fibrosis. Kidney Int, (2017).
20. A. B. Bialkowska, V. W. Yang, S. K. Mallipattu, Kruppel-like factors in mammalian stem cells and development. Development 144, 737-754 (2017).
21. M. J. Rane, Y. Zhao, L. Cai, Krupsilonppel-like factors (KLFs) in renal physiology and disease. EBioMedicine 40, 743-750 (2019).
22. L. Wang, W. Lin, J. Chen, Kruppel-like Factor 15: A Potential Therapeutic Target For Kidney Disease. Int J Biol Sci 15, 1955-1961 (2019).
23. Y. Guo et al., Podocyte-Specific Induction of Kruppel-Like Factor 15 Restores Differentiation Markers and Attenuates Kidney Injury in Proteinuric Kidney Disease. J Am Soc Nephrol 29, 2529-2545 (2018).
24. S. K. Mallipattu et al., Kruppel-like factor 15 (KLF15) is a key regulator of podocyte differentiation. J Biol Chem 287, 19122-19135 (2012).
25. A. B. Bialkowska, Y. Du, H. Fu, V. W. Yang, Identification of novel small-molecule compounds that inhibit the proproliferative Kruppel-like factor 5 in colorectal cancer cells by high-throughput screening. Mol Cancer Ther 8, 563-570 (2009).
26. M. Asada et al., DNA binding-dependent glucocorticoid receptor activity promotes adipogenesis via Kruppel-like factor 15 gene expression. Lab Invest 91, 203-215 (2011).
27. S. K. Sasse et al., The glucocorticoid receptor and KLF15 regulate gene expression dynamics and integrate signals through feed-forward circuitry. Mol Cell Biol 33, 2104-2115 (2013).
28. A. Daina, O. Michielin, V. Zoete, SwissADME: a free web tool to evaluate pharmacokinetics, drug-likeness and medicinal chemistry friendliness of small molecules. Sci Rep 7, 42717 (2017).
29. J. Reiser et al., Induction of B7-1 in podocytes is associated with nephrotic syndrome. J Clin Invest 113, 1390-1397 (2004).
30. H. W. Lee et al., A Podocyte-Based Automated Screening Assay Identifies Protective Small Molecules. J Am Soc Nephrol 26, 2741-2752 (2015).
31. V. A. Rufanova et al., C3G overexpression in glomerular epithelial cells during anti-GBM-induced glomerulonephritis. Kidney Int 75, 31-40 (2009).
32. P. Dickie et al., HIV-associated nephropathy in transgenic mice expressing HIV-1 genes. Virology 185, 109-119 (1991).
33. E. Y. Chen et al., Enrichr: interactive and collaborative HTML5 gene list enrichment analysis tool. BMC Bioinformatics 14, 128 (2013).
34. A. R. Pico et al., WikiPathways: pathway editing for the people. PLoS Biol 6, e184 (2008).
35. D. N. Slenter et al., WikiPathways: a multifaceted pathway database bridging metabolomics to other omics research. Nucleic Acids Res 46, D661-d667 (2018).
36. M. Kanehisa, S. Goto, KEGG: kyoto encyclopedia of genes and genomes. Nucleic Acids Res 28, 27-30 (2000).
37. X. Gao et al., Low-protein diet supplemented with ketoacids reduces the severity of renal disease in 5/6 nephrectomized rats: a role for KLF15. Kidney Int 79, 987-996 (2011).
38. Y. Lu et al., Kruppel-like factor 15 is critical for vascular inflammation. J Clin Invest 123, 4232-4241 (2013).
39. B. Liu et al., Protective effect of KLF15 on vascular endothelial dysfunction induced by TNF-alpha. Mol Med Rep 18, 1987-1994 (2018).
40. A. Lachmann et al., ChEA: transcription factor regulation inferred from integrating genome-wide ChIP-X experiments. Bioinformatics 26, 2438-2444 (2010).
41. A. Israel, The IKK complex, a central regulator of NF-kappaB activation. Cold Spring Harb Perspect Biol 2, a000158 (2010).
42. M. Drexel, J. Kirchmair, S. Santos-Sierra, INH14, a Small-Molecule Urea Derivative, Inhibits the IKKalpha/beta-Dependent TLR Inflammatory Response. Chembiochem 20, 710-717 (2019).
43. M. Ikuta et al., Crystallographic approach to identification of cyclin-dependent kinase 4 (CDK4)-specific inhibitors by using CDK4 mimic CDK2 protein. J Biol Chem 276, 27548-27554 (2001).
44. R. Ronchetti, G. Moroni, A. Carotti, A. Gioiello, E. Camaioni, Recent advances in urea- and thiourea-containing compounds: focus on innovative approaches in medicinal chemistry and organic synthesis. Rsc Med Chem 12, 1046-1064 (2021).
45. S. Ghosh, S. Ghosh, R. Raza, K. Ghosh, Progress of 3-aminopyridine-based amide, urea, imine and azo derivatives in supramolecular gelation. J Indian Chem Soc 99, (2022).
46. R. Listro et al., Urea-based anticancer agents. Exploring 100-years of research with an eye to the future. Front Chem 10, (2022).
47. P. D. M. Pinheiro, D. A. Rodrigues, R. D. Maia, S. Thota, C. A. M. Fraga, The Use of Conformational Restriction in Medicinal Chemistry. Curr Top Med Chem 19, 1712-1733 (2019).
48. S. Kumari, A. V. Carmona, A. K. Tiwari, P. C. Trippier, Amide Bond Bioisosteres: Strategies, Synthesis, and Successes. Journal of Medicinal Chemistry 63, 12290-12358 (2020).
49. E. A. Korte et al., ABIN1 Determines Severity of Glomerulonephritis via Activation of Intrinsic Glomerular Inflammation. Am J Pathol 187, 2799-2810 (2017).
50. N. Prakoura et al., NFkappaB-Induced Periostin Activates Integrin-beta3 Signaling to Promote Renal Injury in GN. J Am Soc Nephrol 28, 1475-1490 (2017).
51. N. Song, F. Thaiss, L. Guo, NFkappaB and Kidney Injury. Front Immunol 10, 815 (2019).
52. S. Brahler et al., Intrinsic proinflammatory signaling in podocytes contributes to podocyte damage and prolonged proteinuria. American Journal of Physiology-Renal Physiology 303, F1473-F1485 (2012).
53. H. Bao, Y. Ge, A. Peng, R. Gong, Fine-tuning of NFκB by glycogen synthase kinase 33 directs the fate of glomerular podocytes upon injury. Kidney Int 87, 1176-1190 (2015).
54. D. J. Caster et al., ABIN1 dysfunction as a genetic basis for lupus nephritis. J Am Soc Nephrol 24, 1743-1754 (2013).
55. J. Y. Zhang et al., UBD modifies APOL1-induced kidney disease risk. Proc Natl Acad Sci USA 115, 3446-3451 (2018).
56. J. I. Lee, G. J. Burckart, Nuclear factor kappa B: important transcription factor and therapeutic target. J Clin Pharmacol 38, 981-993 (1998).
57. L. I. McKay, J. A. Cidlowski, Molecular control of immune/inflammatory responses: interactions between nuclear factor-kappa B and steroid receptor-signaling pathways. Endocr Rev 20, 435-459 (1999).
58. F. T. H. Lee et al., Interactions between angiotensin II and NF-kappa B-dependent pathways in modulating macrophage infiltration in experimental diabetic nephropathy. Journal of the American Society of Nephrology 15, 2139-2151 (2004).
59. R. Patel, J. Williams-Dautovich, C. L. Cummins, Minireview: New Molecular Mediators of Glucocorticoid Receptor Activity in Metabolic Tissues. Mol Endocrinol 28, 999-1011 (2014).
60. M. A. Saleem et al., A conditionally immortalized human podocyte cell line demonstrating nephrin and podocin expression. J Am Soc Nephrol 13, 630-638 (2002).
61. J. M. Ahn, N. A. Boyle, M. T. MacDonald, K. D. Janda, Peptidomimetics and peptide backbone modifications. Mini Rev Med Chem 2, 463-473 (2002).
62. Z. Wang, A. Ma'ayan, An open RNA-Seq data analysis pipeline tutorial with an example of reprocessing data from a recent Zika virus study. F1000Res 5, 1574 (2016).
63. A. Dobin et al., STAR: ultrafast universal RNA-seq aligner. Bioinformatics 29, 15-21 (2013).
64. Y. Liao, G. K. Smyth, W. Shi, featureCounts: an efficient general purpose program for assigning sequence reads to genomic features. Bioinformatics 30, 923-930 (2014).
65. A. Lachmann et al., Massive mining of publicly available RNA-seq data from human and mouse. Nat Commun 9, 1366 (2018).
66. M. V. Kuleshov et al., Enrichr: a comprehensive gene set enrichment analysis web server 2016 update. Nucleic Acids Res 44, W90-97 (2016).
67. A. Subramanian et al., Gene set enrichment analysis: A knowledge-based approach for interpreting genome-wide expression profiles. P Natl Acad Sci USA 102, 15545-15550 (2005).
68. G. C. Yu, L. G. Wang, Y. Y. Han, Q. Y. He, clusterProfiler: an R Package for Comparing Biological Themes Among Gene Clusters. Omics 16, 284-287 (2012).
69. V. Matys et al., TRANSFAC: transcriptional regulation, from patterns to profiles. Nucleic Acids Res 31, 374-378 (2003).
70. V. Matys et al., TRANSFAC and its module TRANSCompel: transcriptional gene regulation in eukaryotes. Nucleic Acids Res 34, D108-110 (2006).
71. C. C. Estrada et al., Kruppel-like factor 4 is a negative regulator of STAT3-induced glomerular epithelial cell proliferation. JCI Insight 3, (2018).
72. K. Suzuki, P. Bose, R. Y. Leong-Quong, D. J. Fujita, K. Riabowol, REAP: A two minute cell fractionation method. BMC Res Notes 3, 294 (2010).
73. S. Liu et al., Crystal structure of a human IkappaB kinase beta asymmetric dimer. J Biol Chem 288, 22758-22767 (2013).
74. E. F. Pettersen et al., UCSF Chimera—a visualization system for exploratory research and analysis. J Comput Chem 25, 1605-1612 (2004).
75. A. Sali, T. L. Blundell, Comparative protein modelling by satisfaction of spatial restraints. J Mol Biol 234, 779-815 (1993).
76. F. Madeira et al., Search and sequence analysis tools services from EMBL-EBI in 2022. Nucleic Acids Res 50, W276-W279 (2022).
77. W. J. Allen et al., DOCK 6: Impact of new features and current docking performance. J Comput Chem 36, 1132-1156 (2015).
78. S. Mukherjee, T. E. Balius, R. C. Rizzo, Docking validation resources: protein family and ligand flexibility experiments. J Chem Inf Model 50, 1986-2000 (2010).
79. A. Jakalian, D. B. Jack, C. I. Bayly, Fast, efficient generation of high-quality atomic charges. AM1-BCC model: II. Parameterization and validation. J Comput Chem 23, 1623-1641 (2002).
80. J. Wang, W. Wang, P. A. Kollman, D. A. Case, Automatic atom type and bond type perception in molecular mechanical calculations. J Mol Graph Model 25, 247-260 (2006).
81. H. M. A. D. A. Case, K. Belfon, I. Y. Ben-Shalom, J. T. Berryman, S. R. Brozell, D. S. Cerutti, T. E. Cheatham, III, G. A. Cisneros, V. W. D. Cruzeiro, T. A. Darden, N. Forouzesh, G. Giamba u, T. Giese, M. K. Gilson, H. Gohlke, A. W. Goetz, J. Harris, S. Izadi, S. A. Izmailov, K. Kasavajhala, M. C. Kaymak, E. King, A. Kovalenko, T. Kurtzman, T. S. Lee, P. Li, C. Lin, J. Liu, T. Luchko, R. Luo, M. Machado, V. Man, M. Manathunga, K. M. Merz, Y. Miao, O. Mikhailovskii, G. Monard, H. Nguyen, K. A. O'Hearn, A. Onufriev, F. Pan, S. Pantano, R. Qi, A. Rahnamoun, D. R. Roe, A. Roitberg, C. Sagui, S. Schott-Verdugo, A. Shajan, J. Shen, C. L. Simmerling, N. R. Skrynnikov, J. Smith, J. Swails, R. C. Walker, J. Wang, J. Wang, H. Wei, X. Wu, Y. Wu, Y. Xiong, Y. Xue, D. M. York, S. Zhao, Q. Zhu, and P. A. Kollman, Amber 2023 University of California, San Francisco. (2023).
82. J. A. Maier et al., ff14SB: Improving the Accuracy of Protein Side Chain and Backbone Parameters from ff99SB. J Chem Theory Comput 11, 3696-3713 (2015).
83. J. Wang, R. M. Wolf, J. W. Caldwell, P. A. Kollman, D. A. Case, Development and testing of a general amber force field. J Comput Chem 25, 1157-1174 (2004).
84. R. L. DesJarlais et al., Using shape complementarity as an initial screen in designing ligands for a receptor binding site of known three-dimensional structure. J Med Chem 31, 722-729 (1988).
85. I. D. Kuntz, J. M. Blaney, S. J. Oatley, R. Langridge, T. E. Ferrin, A geometric approach to macromolecule-ligand interactions. J Mol Biol 161, 269-288 (1982).
86. B. K. Shoichet, D. L. Bodian, I. D. Kuntz, Molecular Docking Using Shape Descriptors. Journal of Computational Chemistry 13, 380-397 (1992).
87. T. E. Balius, W. J. Allen, S. Mukherjee, R. C. Rizzo, Grid-based molecular footprint comparison method for docking and de novo design: application to HIVgp41. J Comput Chem 34, 1226-1240 (2013).
88. W. J. Allen, R. C. Rizzo, Implementation of the Hungarian algorithm to account for ligand symmetry and similarity in structure-based design. J Chem Inf Model 54, 518-529 (2014).
89. G. M. Sastry, S. L. Dixon, W. Sherman, Rapid shape-based ligand alignment and virtual screening method based on atom/feature-pair similarities and volume overlap scoring. J Chem Inf Model 51, 2455-2466 (2011).
90. T. E. Balius, S. Mukherjee, R. C. Rizzo, Implementation and evaluation of a docking-rescoring method using molecular footprint comparisons. J Comput Chem 32, 2273-2289 (2011).
91. W. L. Jorgensen, J. Chandrasekhar, J. D. Madura, R. W. Impey, M. L. Klein, Comparison of Simple Potential Functions for Simulating Liquid Water. J Chem Phys 79, 926-935 (1983).
92. Y. C. Zhou et al., Identification of Fatty Acid Binding Protein 5 Inhibitors Through Similarity-Based Screening. Biochemistry-Us 58, 4304-4316 (2019).
93. R. Salomon-Ferrer, A. W. Gotz, D. Poole, S. Le Grand, R. C. Walker, Routine Microsecond Molecular Dynamics Simulations with AMBER on GPUs. 2. Explicit Solvent Particle Mesh Ewald. Journal of Chemical Theory and Computation 9, 3878-3888 (2013).
94. D. R. Roe, T. E. Cheatham, PTRAJ and CPPTRAJ: Software for Processing and Analysis of Molecular Dynamics Trajectory Data. Journal of Chemical Theory and Computation 9, 3084-3095 (2013).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating a disorder associated with overactivation of IKKβ in a subject in need thereof, the method comprising administering to the subject a compound having a structure represented by a formula selected from: 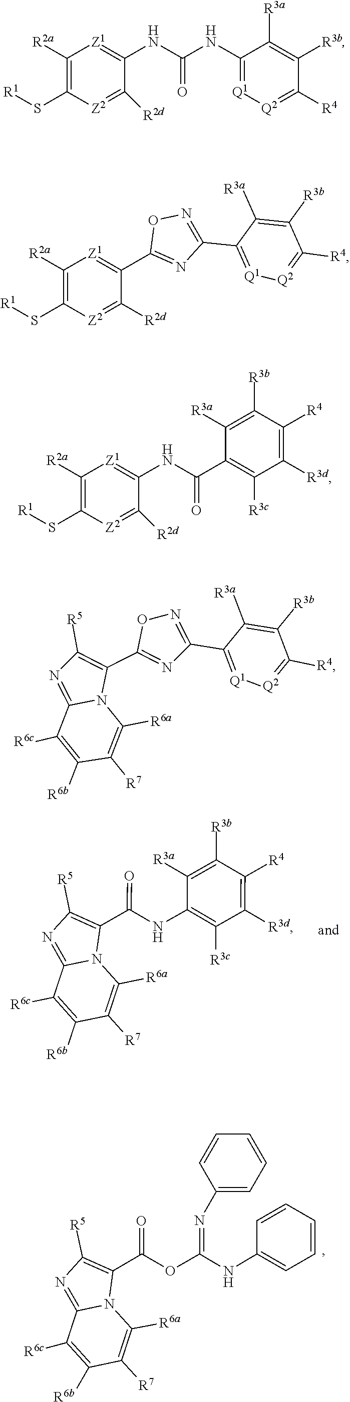
wherein each of Q¹ and Q² is independently selected from N and CR¹⁰;
wherein R¹⁰, when present, is selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein Z¹, when present, is selected from N and CR²b;
wherein Z², when present, is selected from N and CR^{2c};
wherein R¹, when present, is C1-C4 alkyl;
wherein each of R^{2a}, R^{2b}, R^{2c}, R^{2d}, R^{3a}, and R^{3b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R^{3c} and R^{3d}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein R⁴ is independently selected from halogen, —CN, —OH, C1-C4 alkyl, C1-C4 alkoxy, —B(OR¹¹)₂, and —B(R¹²)₃;
wherein each occurrence of R¹¹, when present, is independently selected from hydrogen and C1-C8 alkyl,
or wherein each occurrence of R¹¹, when present, is covalently bonded together, and, together with the intermediate atoms, comprise a C6 bicyclic heterocycle or a C2-C3 heterocycloalkyl, and is substituted with 0, 1, 2, 3, or 4 C1-C4 alkyl groups;
wherein each occurrence of R¹², when present, is independently halogen;
wherein each of R⁵, R^{6a}, R^{6b}, and R^{6c}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein R⁷, when present, is halogen;
or a pharmaceutically acceptable salt thereof, wherein the disorder is selected from cancer, arthritis, a cardiometabolic disease, and chronic obstructive pulmonary disease (COPD).

2. The method of claim 1, wherein the compound has a structure represented by a formula selected from: 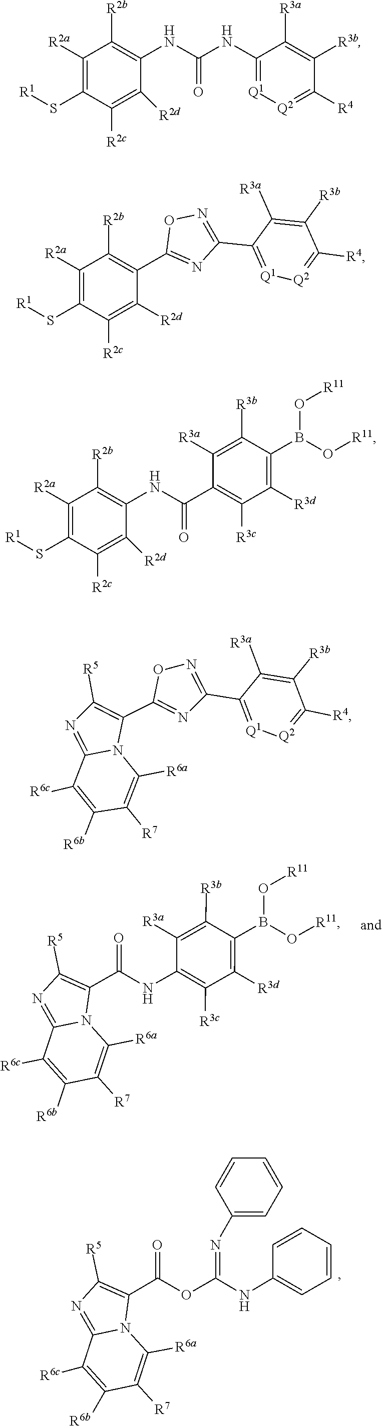
or a pharmaceutically acceptable salt thereof, wherein R⁴, when present, is independently selected from halogen, —CN, —OH, C1-C4 alkyl, C1-C4 alkoxy, and —B(OR¹¹)₂.

3. The method of claim 1, wherein each of R^{2a}, R^{2b}, R^{2c}, R^{2d}, R^{3a}, and R^{3b}, when present, is hydrogen.

4. The method of claim 1, wherein R⁴, when present, is —B(OR¹¹)₂.

5. The method of claim 1, wherein each occurrence of R¹¹, when present, is independently selected from hydrogen and C1-C8 alkyl.

6. The method of claim 1, wherein each occurrence of R¹¹, when present, is covalently bonded together, and, together with the intermediate atoms, comprise a C6 bicyclic heterocycle or a C2-C3 heterocycloalkyl, and is substituted with 0, 1, 2, 3, or 4 C1-C4 alkyl groups.

7. The method of claim 1, wherein R⁷, when present, is —Cl.

8. The method of claim 1, wherein the compound has a structure represented by a formula selected from: 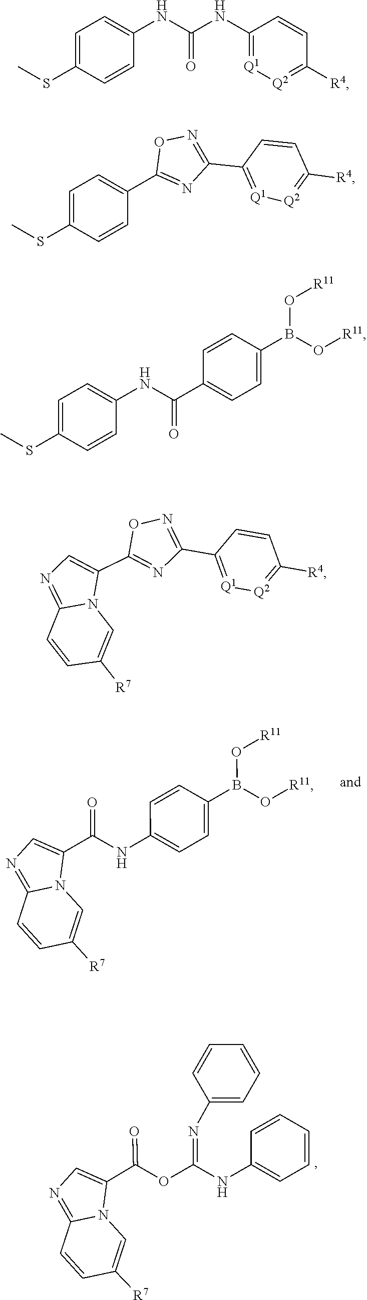
or a pharmaceutically acceptable salt thereof.

9. The method of claim 1, wherein the compound has a structure represented by a formula selected from: 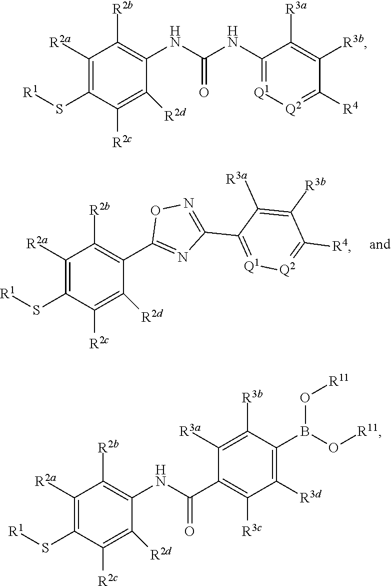
or a pharmaceutically acceptable salt thereof.

10. The method of claim 9, wherein the compound has a structure represented by a formula selected from: 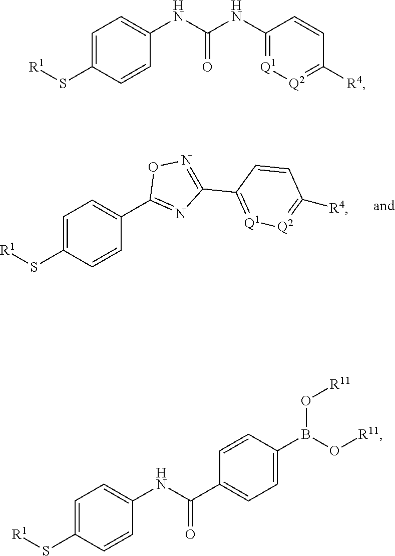
or a pharmaceutically acceptable salt thereof.

11. The method of claim 1, wherein the compound has a structure represented by a formula selected from: 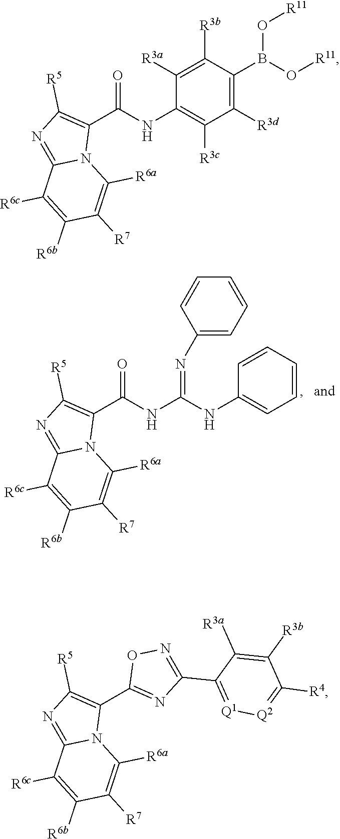
or a pharmaceutically acceptable salt thereof.

12. The method of claim 11, wherein the compound has a structure represented by a formula selected from: 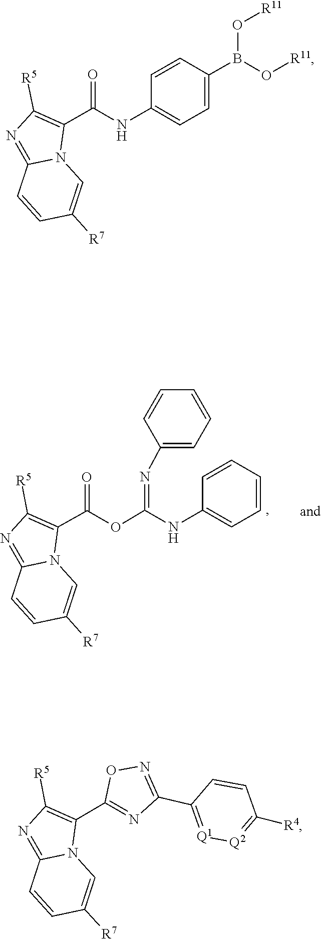
or a pharmaceutically acceptable salt thereof.

13. The method of claim 1, wherein the compound is selected from: 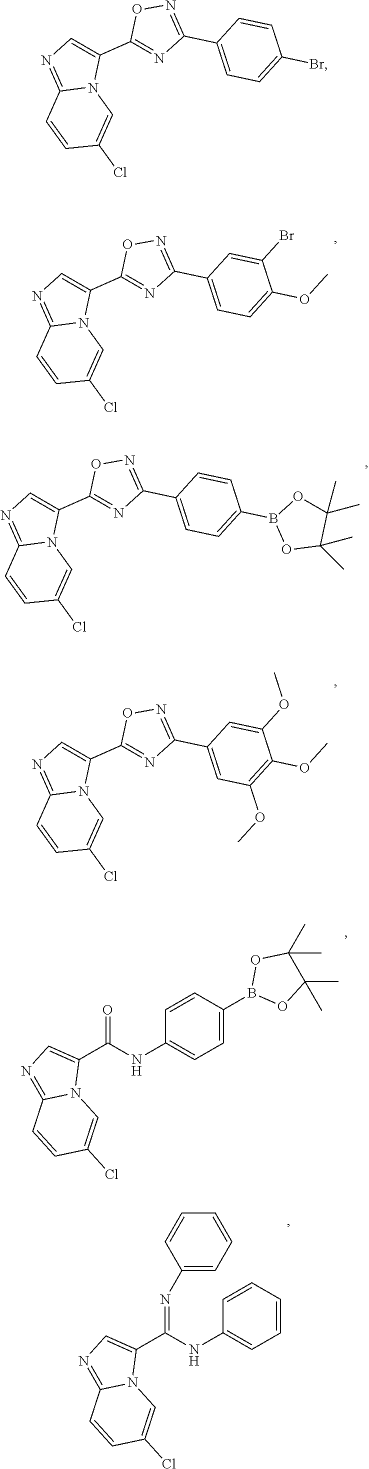 [SMILES: Clc1ccc2ncc(-c3nc(-c4ccc(Br)cc4)no3)n2c1 ; COc1ccc(-c2noc(-c3cnc4ccc(Cl)cn34)n2)cc1Br ; CC1(C)OB(c2ccc(-c3noc(-c4cnc5ccc(Cl)cn45)n3)cc2)OC1(C)C ; COc1cc(-c2noc(-c3cnc4ccc(Cl)cn34)n2)cc(OC)c1OC ; CC1(C)OB(c2ccc(NC(=O)c3cnc4ccc(Cl)cn34)cc2)OC1(C)C ; Clc1ccc2ncc(/C(=N/c3ccccc3)Nc3ccccc3)n2c1] 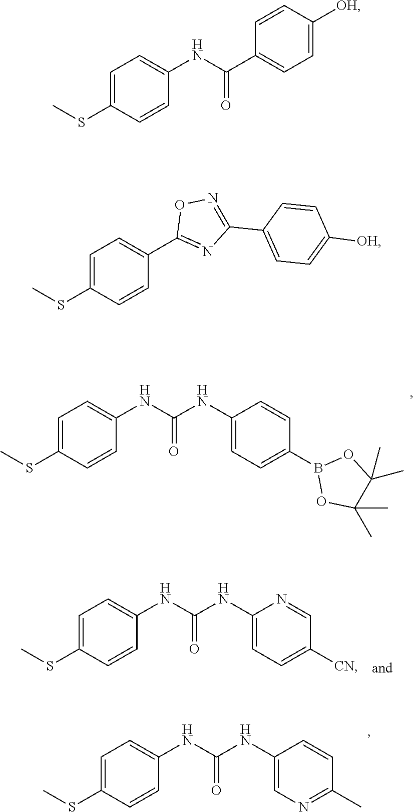
or a pharmaceutically acceptable salt thereof.

14. The method of claim 1, wherein the disorder is cancer.

15. The method of claim 1, wherein the disorder is arthritis.

16. The method of claim 1, wherein the disorder is a cardiometabolic disease.

17. The method of claim 1, wherein the disorder is COPD.

18. The method of claim 1, further comprising administering to the subject an effective amount of a glucocorticoid.

19. A method of treating a disorder associated with overactivation of IKKβ in a subject in need thereof, the method comprising administering to the subject a compound selected from: 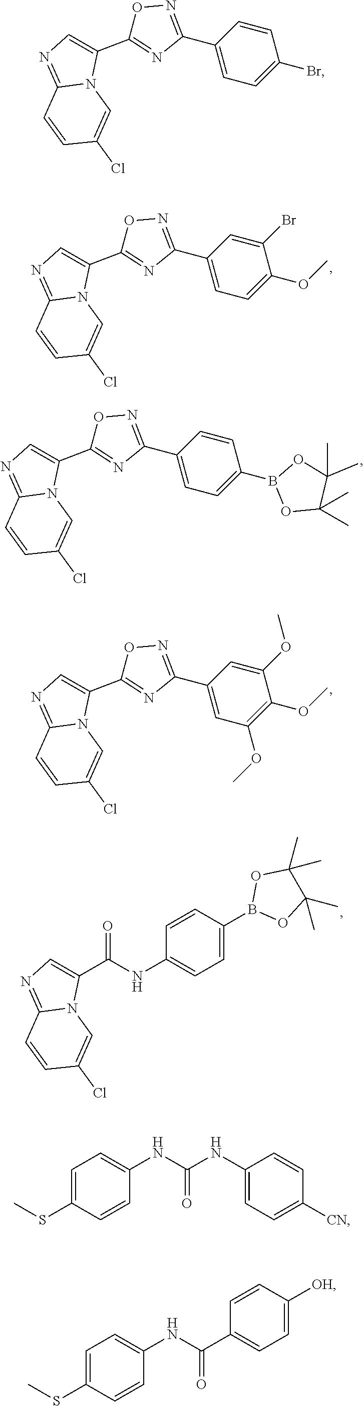 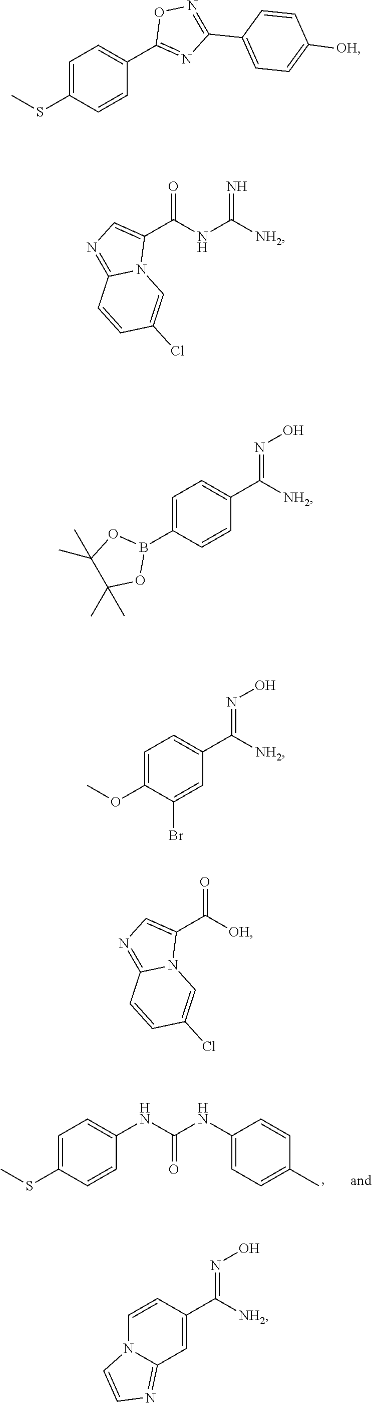
or a pharmaceutically acceptable salt thereof, wherein the disorder is selected from cancer, arthritis, a cardiometabolic disease, and chronic obstructive pulmonary disease (COPD).

20. The method of claim 19, wherein the disorder is selected from a solid tumor, a metastatic melanoma, a hematological malignancy, osteoarthritis, and COPD.
